SEQUENCE LISTING (18 sequences)

SEQ ID NO: 1 - protein, 698 aa, Homo sapiens
MQAHELFRYFRMPELVDFRQYVRTLPTNTLMGFGAFAALTTFWYATRPKPLKPPCDLSMQSVEVAGSGGARRSALLDSDEPLVYFYDDVTTLYEGFQRGIQVSNNGPCLGSRKPDQPYEWLSYKQVAELSECIGSALIQKGFKTAPDQFIGIFAQNRPEWVIIEQGCFAYSMVIVPLYDTLGNEAITYIVNKAELSLVFVDKPEKAKLLLEGVENKLIPGLKIIVVMDAYGSELVERGQRCGVEVTSMKAMEDLGRANRRKPKPPAPEDLAVICFTSGTTGNPKGAMVTHRNIVSDCSAFVKATENTVNPCPDDTLISFLPLAHMFERVVECVMLCHGAKIGFFQGDIRLLMDDLKVLQPTVFPVVPRLLNRMFDRIFGQANTTLKRWLLDFASKRKEAELRSGIIRNNSLWDRLIFHKVQSSLGGRVRLMVTGAAPVSATVLTFLRAALGCQFYEGYGQTECTAGCCLTMPGDWTAGHVGAPMPCNLIKLVDVEEMNYMAAEGEGEVCVKGPNVFQGYLKDPAKTAEALDKDGWLHTGDIGKWLPNGTLKIIDRKKHIFKLAQGEYIAPEKIENIYMRSEPVAQVFVHGESLQAFLIAIVVPDVETLCSWAQKRGFEGSFEELCRNKDVKKAILEDMVRLGKDSGLKPFEQVKGITLHPELFSIDNGLLTPTMKAKRPELRNYFRSQIDDLYSTIKV

SEQ ID NO: 2 - DNA, 2094 bp, Homo sapiens
atgcaagcccatgagctgttccggtattttcgaatgccagagctggttgacttccgacagtacgtgcgtactcttccgaccaacacgcttatgggcttcggagcttttgcagcactcaccaccttctggtacgccacgagacccaaacccctgaagccgccatgcgacctctccatgcagtcagtggaagtggcgggtagtggtggtgcacgaagatccgcactacttgacagcgacgagcccttggtgtatttctatgatgatgtcacaacattatacgaaggtttccagaggggaatacaggtgtcaaataatggcccttgtttaggctctcggaaaccagaccaaccctatgaatggctttcatataaacaggttgcagaattgtcggagtgcataggctcagcactgatccagaagggcttcaagactgccccagatcagttcattggcatctttgctcaaaatagacctgagtgggtgattattgaacaaggatgctttgcttattcgatggtgatcgttccactttatgatacccttggaaatgaagccatcacgtacatagtcaacaaagctgaactctctctggtttttgttgacaagccagagaaggccaaactcttattagagggtgtagaaaataagttaataccaggccttaaaatcatagttgtcatggatgcctacggcagtgaactggtggaacgaggccagaggtgtggggtggaagtcaccagcatgaaggcgatggaggacctgggaagagccaacagacggaagcccaagcctccagcacctgaagatcttgcagtaatttgtttcacaagtggaactacaggcaaccccaaaggagcaatggtcactcaccgaaacatagtgagcgattgttcagcttttgtgaaagcaacagagaatacagtcaatccttgcccagatgatactttgatatctttcttgcctctcgcccatatgtttgagagagttgtagagtgtgtaatgctgtgtcatggagctaaaatcggatttttccaaggagatatcaggctgctcatggatgacctcaaggtgcttcaacccactgtcttccccgtggttccaagactgctgaaccggatgtttgaccgaattttcggacaagcaaacaccacgctgaagcgatggctcttggactttgcctccaagaggaaagaagcagagcttcgcagcggcatcatcagaaacaacagcctgtgggaccggctgatcttccacaaagtacagtcgagcctgggcggaagagtccggctgatggtgacaggagccgccccggtgtctgccactgtgctgacgttcctcagagcagccctgggctgtcagttttatgaaggatacggacagacagagtgcactgccgggtgctgcctgaccatgcctggagactggaccgcaggccatgttggggccccgatgccgtgcaatttgataaaacttgttgatgtggaagaaatgaattacatggctgccgagggcgagggcgaggtgtgtgtgaaagggccaaatgtatttcagggctacttgaaggacccagcgaaaacagcagaagctttggacaaagacggctggttacacacaggggacattggaaaatggttaccaaatggcaccttgaaaattatcgaccggaaaaagcacatatttaagctggcacaaggagaatacatagcccctgaaaagattgaaaatatctacatgcgaagtgagcctgttgctcaggtgtttgtccacggagaaagcctgcaggcatttctcattgcaattgtggtaccagatgttgagacattatgttcctgggcccaaaagagaggatttgaagggtcgtttgaggaactgtgcagaaataaggatgtcaaaaaagctatcctcgaagatatggtgagacttgggaaggattctggtctgaaaccatttgaacaggtcaaaggcatcacattgcaccctgaattattttctatcgacaatggccttctgactccaacaatgaaggcgaaaaggccagagctgcggaactatttcaggtcgcagatagatgacctctattccactatcaaggtt

SEQ ID NO: 3 - protein, 720 aa, Homo sapiens
MNNHVSSKPSTMKLKHTINPILLYFIHFLISLYTILTYIPFYFFSESRQEKSNRIKAKPVNSKPDSAYRSVNSLDGLASVLYPGCDTLDKVFTYAKNKFKNKRLLGTREVLNEEDEVQPNGKIFKKVILGQYNWLSYEDVFVRAFNFGNGLQMLGQKPKTNIAIFCETRAEWMIAAQACFMYNFQLVTLYATLGGPAIVHALNETEVTNIITSKELLQTKLKDIVSLVPRLRHIITVDGKPPTWSEFPKGIIVHTMAAVEALGAKASMENQPHSKPLPSDIAVIMYTSGSTGLPKGVMISHSNIIAGITGMAERIPELGEEDVYIGYLPLAHVLELSAELVCLSHGCRIGYSSPQTLADQSSKIKKGSKGDTSMLKPTLMAAVPEIMDRIYKNVMNKVSEMSSFQRNLFILAYNYKMEQISKGRNTPLCDSFVFRKVRSLLGGNIRLLLCGGAPLSATTQRFMNICFCCPVGQGYGLTESAGAGTISEVWDYNTGRVGAPLVCCEIKLKNWEEGGYFNTDKPHPRGEILIGGQSVTMGYYKNEAKTKADFFEDENGQRWLCTGDIGEFEPDGCLKIIDRKKDLVKLQAGEYVSLGKVEAALKNLPLVDNICAYANSYHSYVIGFVVPNQKELTELARKKGLKGTWEELCNSCEMENEVLKVLSEAAISASLEKFEIPVKIRLSPEPWTPETGLVTDAFKLKRKELKTHYQADIERMYGRK

SEQ ID NO: 4 - DNA, 2160 bp, Homo sapiens
atgaataaccacgtgtcttcaaaaccatctaccatgaagctaaaacataccatcaaccctattcttttatattttatacattttctaatatcactttatactattttaacatacattccgttttattttttctccgagtcaagacaagaaaaatcaaaccgaattaaagcaaagcctgtaaattcaaaacctgattctgcatacagatctgttaatagtttggatggtttggcttcagtattataccctggatgtgatactttagataaagtttttacatatgcaaaaaacaaatttaagaacaaaagactcttgggaacacgtgaagttttaaatgaggaagatgaagtacaaccaaatggaaaaatttttaaaaaggttattcttggacagtataattggctttcctatgaagatgtctttgttcgagcctttaattttggaaatggattacagatgttgggtcagaaaccaaagaccaacatcgccatcttctgtgagaccagggccgagtggatgatagctgcacaggcgtgttttatgtataattttcagcttgttacattatatgccactctaggaggtccagccattgttcatgcattaaatgaaacagaggtgaccaacatcattactagtaaagaactcttacaaacaaagttgaaggatatagtttctttggtcccacgcctgcggcacatcatcactgttgatggaaagccaccgacctggtccgagttccccaagggcatcattgtgcataccatggctgcagtggaggccctgggagccaaggccagcatggaaaaccaacctcatagcaaaccattgccctcagatattgcagtaatcatgtacacaagtggatccacaggacttccaaagggagtcatgatctcacatagtaacattattgctggtataactgggatggcagaaaggattccagaactaggagaggaagatgtctacattggatatttgcctctggcccatgttctagaattaagtgctgagcttgtctgtctttctcacggatgccgcattggttactcttcaccacagactttagcagatcagtcttcaaaaattaaaaaaggaagcaaaggggatacatccatgttgaaaccaacactgatggcagcagttccggaaatcatggatcggatctacaaaaatgtcatgaataaagtcagtgaaatgagtagttttcaacgtaatctgtttattctggcctataattacaaaatggaacagatttcaaaaggacgtaatactccactgtgcgacagctttgttttccggaaagttcgaagcttgctagggggaaatattcgtctcctgttgtgtggtggcgctccactttctgcaaccacgcagcgattcatgaacatctgtttctgctgtcctgttggtcagggatacgggctcactgaatctgctggggctggaacaatttccgaagtgtgggactacaatactggcagagtgggagcaccattagtttgctgtgaaatcaaattaaaaaactgggaggaaggtggatactttaatactgataagccacaccccaggggtgaaattcttattgggggccaaagtgtgacaatggggtactacaaaaatgaagcaaaaacaaaagctgatttctttgaagatgaaaatggacaaaggtggctctgtactggggatattggagagtttgaacccgatggatgcttaaagattattgatcgtaaaaaggaccttgtaaaactacaggcaggggaatatgtttctcttgggaaagtagaggcagctttgaagaatcttccactagtagataacatttgtgcatatgcaaacagttatcattcttatgtcattggatttgttgtgccaaatcaaaaggaactaactgaactagctcgaaagaaaggacttaaagggacttgggaggagctgtgtaacagttgtgaaatggaaaatgaggtacttaaagtgctttccgaagctgctatttcagcaagtctggaaaagtttgaaattccagtaaaaattcgtttgagtcctgaaccgtggacccctgaaactggtctggtgacagatgccttcaagctgaaacgcaaagagcttaaaacacattaccaggcggacattgagcgaatgtatggaagaaaa

SEQ ID NO: 5 - protein, 670 aa, Homo sapiens
MAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 6 - DNA, 2010 bp, Homo sapiens
atggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaa

SEQ ID NO: 7 - protein, 711 aa, Homo sapiens
MKLKLNVLTIILLPVHLLITIYSALIFIPWYFLTNAKKKNAMAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 8 - DNA, 2133 bp, Homo sapiens
atgaaacttaagctaaatgtgctcaccattattttgctgcctgtccacttgttaataacaatatacagtgcccttatatttattccatggtattttcttaccaatgccaagaagaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaa

SEQ ID NO: 9 - protein, 739 aa, Homo sapiens
MDALKPPCLWRNHERGKKDRDSCGRKNSEPGSPHSLEALRDAAPSQGLNFLLLFTKMLFIFNFLFSPLPTPALICILTFGAAIFLWLITRPQPVLPLLDLNNQSVGIEGGARKGVSQKNNDLTSCCFSDAKTMYEVFQRGLAVSDNGPCLGYRKPNQPYRWLSYKQVSDRAEYLGSCLLHKGYKSSPDQFVGIFAQNRPEWIISELACYTYSMVAVPLYDTLGPEAIVHIVNKADIAMVICDTPQKALVLIGNVEKGFTPSLKVIILMDPFDDDLKQRGEKSGIEILSLYDAENLGKEHFRKPVPPSPEDLSVICFTSGTTGDPKGAMITHQNIVSNAAAFLKCVEHAYEPTPDDVAISYLPLAHMFERIVQAVVYSCGARVGFFQGDIRLLADDMKTLKPTLFPAVPRLLNRIYDKVQNEAKTPLKKFLLKLAVSSKFKELQKGIIRHDSFWDKLIFAKIQDSLGGRVRVIVTGAAPMSTSVMTFFRAAMGCQVYEAYGQTECTGGCTFTLPGDWTSGHVGVPLACNYVKLEDVADMNYFTVNNEGEVCIKGTNVFKGYLKDPEKTQEALDSDGWLHTGDIGRWLPNGTLKIIDRKKNIFKLAQGEYIAPEKIENIYNRSQPVLQIFVHGESLRSSLVGVVVPDTDVLPSFAAKLGVKGSFEELCQNQVVREAILEDLQKIGKESGLKTFEQVKAIFLHPEPFSIENGLLTPTLKAKRGELSKYFRTQIDSLYEHIQD

SEQ ID NO: 10 - DNA, 2217 bp, Homo sapiens
atggacgctctgaagccaccctgtctctggaggaaccacgagcgagggaagaaggacagggactcgtgtggcaggaagaactcagagccgggaagcccccattcactagaagcactgagagatgcggccccctcgcagggtctgaatttcctgctgctgttcacaaagatgctttttatctttaactttttgttttccccacttccgaccccggcgttgatctgcatcctgacatttggagctgccatcttcttgtggctgatcaccagacctcaacccgtcttacctcttcttgacctgaacaatcagtctgtgggaattgagggaggagcacggaagggggtttcccagaagaacaatgacctaacaagttgctgcttctcagatgccaagactatgtatgaggttttccaaagaggactcgctgtgtctgacaatgggccctgcttgggatatagaaaaccaaaccagccctacagatggctatcttacaaacaggtgtctgatagagcagagtacctgggttcctgtctcttgcataaaggttataaatcatcaccagaccagtttgtcggcatctttgctcagaataggccagagtggatcatctccgaattggcttgttacacgtactctatggtagctgtacctctgtatgacaccttgggaccagaagccatcgtacatattgtcaacaaggctgatatcgccatggtgatctgtgacacaccccaaaaggcattggtgctgatagggaatgtagagaaaggcttcaccccgagcctgaaggtgatcatccttatggacccctttgatgatgacctgaagcaaagaggggagaagagtggaattgagatcttatccctatatgatgctgagaacctaggcaaagagcacttcagaaaacctgtgcctcctagcccagaagacctgagcgtcatctgcttcaccagtgggaccacaggtgaccccaaaggagccatgataacccatcaaaatattgtttcaaatgctgctgcctttctcaaatgtgtggagcatgcttatgagcccactcctgatgatgtggccatatcctacctccctctggctcatatgtttgagaggattgtacaggctgttgtgtacagctgtggagccagagttggattcttccaaggggatattcggttgctggctgacgacatgaagactttgaagcccacattgtttcccgcggtgcctcgactccttaacaggatctacgataaggtacaaaatgaggccaagacacccttgaagaagttcttgttgaagctggctgtttccagtaaattcaaagagcttcaaaagggtatcatcaggcatgatagtttctgggacaagctcatctttgcaaagatccaggacagcctgggcggaagggttcgtgtaattgtcactggagctgcccccatgtccacttcagtcatgacattcttccgggcagcaatgggatgtcaggtgtatgaagcttatggtcaaacagaatgcacaggtggctgtacatttacattacctggggactggacatcaggtcacgttggggtgcccctggcttgcaattacgtgaagctggaagatgtggctgacatgaactactttacagtgaataatgaaggagaggtctgcatcaagggtacaaacgtgttcaaaggatacctgaaggaccctgagaagacacaggaagccctggacagtgatggctggcttcacacaggagacattggtcgctggctcccgaatggaactctgaagatcatcgaccgtaaaaagaacattttcaagctggcccaaggagaatacattgcaccagagaagatagaaaatatctacaacaggagtcaaccagtgttacaaatttttgtacacggggagagcttacggtcatccttagtaggagtggtggttcctgacacagatgtacttccctcatttgcagccaagcttggggtgaagggctcctttgaggaactgtgccaaaaccaagttgtaagggaagccattttagaagacttgcagaaaattgggaaagaaagtggccttaaaacttttgaacaggtcaaagccatttttcttcatccagagccattttccattgaaaatgggctcttgacaccaacattgaaagcaaagcgaggagagctttccaaatactttcggacccaaattgacagcctgtatgagcacatccaggat

SEQ ID NO: 11 - protein, 683 aa, Homo sapiens
MLFIFNFLFSPLPTPALICILTFGAAIFLWLITRPQPVLPLLDLNNQSVGIEGGARKGVSQKNNDLTSCCFSDAKTMYEVFQRGLAVSDNGPCLGYRKPNQPYRWLSYKQVSDRAEYLGSCLLHKGYKSSPDQFVGIFAQNRPEWIISELACYTYSMVAVPLYDTLGPEAIVHIVNKADIAMVICDTPQKALVLIGNVEKGFTPSLKVIILMDPFDDDLKQRGEKSGIEILSLYDAENLGKEHFRKPVPPSPEDLSVICFTSGTTGDPKGAMITHQNIVSNAAAFLKCVEHAYEPTPDDVAISYLPLAHMFERIVQAVVYSCGARVGFFQGDIRLLADDMKTLKPTLFPAVPRLLNRIYDKVQNEAKTPLKKFLLKLAVSSKFKELQKGIIRHDSFWDKLIFAKIQDSLGGRVRVIVTGAAPMSTSVMTFFRAAMGCQVYEAYGQTECTGGCTFTLPGDWTSGHVGVPLACNYVKLEDVADMNYFTVNNEGEVCIKGTNVFKGYLKDPEKTQEALDSDGWLHTGDIGRWLPNGTLKIIDRKKNIFKLAQGEYIAPEKIENIYNRSQPVLQIFVHGESLRSSLVGVVVPDTDVLPSFAAKLGVKGSFEELCQNQVVREAILEDLQKIGKESGLKTFEQVKAIFLHPEPFSIENGLLTPTLKAKRGELSKYFRTQIDSLYEHIQD

SEQ ID NO: 12 - DNA, 2049 bp, Homo sapiens
atgctttttatctttaactttttgttttccccacttccgaccccggcgttgatctgcatcctgacatttggagctgccatcttcttgtggctgatcaccagacctcaacccgtcttacctcttcttgacctgaacaatcagtctgtgggaattgagggaggagcacggaagggggtttcccagaagaacaatgacctaacaagttgctgcttctcagatgccaagactatgtatgaggttttccaaagaggactcgctgtgtctgacaatgggccctgcttgggatatagaaaaccaaaccagccctacagatggctatcttacaaacaggtgtctgatagagcagagtacctgggttcctgtctcttgcataaaggttataaatcatcaccagaccagtttgtcggcatctttgctcagaataggccagagtggatcatctccgaattggcttgttacacgtactctatggtagctgtacctctgtatgacaccttgggaccagaagccatcgtacatattgtcaacaaggctgatatcgccatggtgatctgtgacacaccccaaaaggcattggtgctgatagggaatgtagagaaaggcttcaccccgagcctgaaggtgatcatccttatggacccctttgatgatgacctgaagcaaagaggggagaagagtggaattgagatcttatccctatatgatgctgagaacctaggcaaagagcacttcagaaaacctgtgcctcctagcccagaagacctgagcgtcatctgcttcaccagtgggaccacaggtgaccccaaaggagccatgataacccatcaaaatattgtttcaaatgctgctgcctttctcaaatgtgtggagcatgcttatgagcccactcctgatgatgtggccatatcctacctccctctggctcatatgtttgagaggattgtacaggctgttgtgtacagctgtggagccagagttggattcttccaaggggatattcggttgctggctgacgacatgaagactttgaagcccacattgtttcccgcggtgcctcgactccttaacaggatctacgataaggtacaaaatgaggccaagacacccttgaagaagttcttgttgaagctggctgtttccagtaaattcaaagagcttcaaaagggtatcatcaggcatgatagtttctgggacaagctcatctttgcaaagatccaggacagcctgggcggaagggttcgtgtaattgtcactggagctgcccccatgtccacttcagtcatgacattcttccgggcagcaatgggatgtcaggtgtatgaagcttatggtcaaacagaatgcacaggtggctgtacatttacattacctggggactggacatcaggtcacgttggggtgcccctggcttgcaattacgtgaagctggaagatgtggctgacatgaactactttacagtgaataatgaaggagaggtctgcatcaagggtacaaacgtgttcaaaggatacctgaaggaccctgagaagacacaggaagccctggacagtgatggctggcttcacacaggagacattggtcgctggctcccgaatggaactctgaagatcatcgaccgtaaaaagaacattttcaagctggcccaaggagaatacattgcaccagagaagatagaaaatatctacaacaggagtcaaccagtgttacaaatttttgtacacggggagagcttacggtcatccttagtaggagtggtggttcctgacacagatgtacttccctcatttgcagccaagcttggggtgaagggctcctttgaggaactgtgccaaaaccaagttgtaagggaagccattttagaagacttgcagaaaattgggaaagaaagtggccttaaaacttttgaacaggtcaaagccatttttcttcatccagagccattttccattgaaaatgggctcttgacaccaacattgaaagcaaagcgaggagagctttccaaatactttcggacccaaattgacagcctgtatgagcacatccaggat

SEQ ID NO: 13 - protein, 722 aa, Homo sapiens
MLTFFLVSGGSLWLFVEFVLSLLEKMQTQEILRILRLPELGDLGQFFRSLSATTLVSMGALAAILAYWFTHRPKALQPPCNLLMQSEEVEDSGGARRSVIGSGPQLLTHYYDDARTMYQVFRRGLSISGNGPCLGFRKPKQPYQWLSYQEVADRAEFLGSGLLQHNCKACTDQFIGVFAQNRPEWIIVELACYTYSMVVVPLYDTLGPGAIRYIINTADISTVIVDKPQKAVLLLEHVERKETPGLKLIILMDPFEEALKERGQKCGVVIKSMQAVEDCGQENHQAPVPPQPDDLSIVCFTSGTTGNPKGAMLTHGNVVADFSGFLKVTESQWAPTCADVHISYLPLAHMFERMVQSVVYCHGGRVGFFQGDIRLLSDDMKALCPTIFPVVPRLLNRMYDKIFSQANTPLKRWLLEFAAKRKQAEVRSGIIRNDSIWDELFFNKIQASLGGCVRMIVTGAAPASPTVLGFLRAALGCQVYEGYGQTECTAGCTFTTPGDWTSGHVGAPLPCNHIKLVDVEELNYWACKGEGEICVRGPNVFKGYLKDPDRTKEALDSDGWLHTGDIGKWLPAGTLKIIDRKKHIFKLAQGEYVAPEKIENIYIRSQPVAQIYVHGDSLKAFLVGIVVPDPEVMPSWAQKRGIEGTYADLCTNKDLKKAILEDMVRLGKESGLHSFEQVKAIHIHSDMFSVQNGLLTPTLKAKRPELREYFKKQIEELYSISM

SEQ ID NO: 14 - DNA, 2166 bp, Homo sapiens
atgctgaccttcttcctcgtgtcggggggctccctctggctattcgtagagtttgtcctctcacttctggagaagatgcagacacaggagatcctgaggatactgcgactgcctgagctaggtgacttgggacagtttttccgcagcctctcggccaccaccctcgtgagtatgggtgccctggctgccatccttgcctactggttcactcaccggccaaaggccttgcagccgccatgcaacctcctgatgcagtcagaagaagtagaggacagtggcggggcacggcgatctgtgattgggtctggccctcagctacttacccactactatgatgatgcccggaccatgtaccaggtgttccgccgtgggcttagcatctcagggaatgggccctgtcttggtttcaggaagcctaagcagccttaccagtggctgtcctaccaggaggtggccgacagggctgaatttctggggtccggacttctccagcacaattgtaaagcatgcactgatcagtttattggtgtttttgcacaaaatcggccagagtggatcattgtggagctggcctgctacacatattccatggtggtggtcccgctctatgacaccctgggccctggggctatccgctacatcatcaatacagcggacatcagcaccgtgattgtggacaaacctcagaaggctgtgcttctgctagagcatgtggagaggaaggagactccaggcctcaagctgatcatcctcatggacccattcgaagaagccctgaaagagagagggcagaagtgcggggtggtcattaagtccatgcaggccgtggaggactgtggccaagagaatcaccaggctcctgtgcccccgcagcctgatgacctctccattgtgtgtttcacaagcggcacgacagggaacccaaaaggtgcgatgctcacccatgggaacgtggtggctgatttctcaggctttctgaaagtgacagagagtcagtgggctcccacttgtgcggatgtgcacatttcctatttgcctttagcacacatgtttgagcgaatggtgcagtctgtcgtctattgccacggagggcgtgttggcttcttccagggagatatccgccttctctcagatgacatgaaggctctatgccccaccatcttccctgtggtcccacgactgctgaaccggatgtacgacaagatcttcagccaggcaaacacaccattaaagcgctggctcctggagtttgcagcaaagcgtaagcaagccgaggtccggagtggaatcatcaggaatgatagtatctgggatgaactcttctttaataagattcaggccagtcttggtgggtgtgtgcggatgattgttactggagcagccccagcatcaccaacagttctgggatttctccgggcagctctagggtgccaggtttatgaaggttatggccaaactgagtgcacagctggatgtaccttcaccactcctggcgactggacctcagggcacgtaggggcgccacttccctgcaatcatatcaagctcgttgatgttgaggaactgaactactgggcctgcaaaggagagggagagatatgtgtgagaggaccaaatgtgttcaaaggctacttgaaagatccagacaggacgaaggaggccctggacagcgatggctggcttcacactggagacatcggaaaatggctgccggcaggaactcttaaaattattgatcggaaaaagcatatatttaaacttgctcagggagaatatgttgcacccgagaagattgagaacatctacatccggagccaacctgtggcgcaaatctatgtccatggggacagcttaaaggcctttttggtaggcattgttgtgcctgaccctgaagttatgccctcctgggcccagaagagaggaattgaaggaacatatgcagatctctgcacaaataaggatctgaagaaagccattttggaagatatggtgaggttaggaaaagaaagtggactccattcttttgagcaggttaaagccattcacatccattctgacatgttctcagttcaaaatggcttgctgacaccaacactaaaagctaagagacctgagctgagagagtacttcaaaaaacaaatagaagagctttactcaatctccatg

SEQ ID NO: 15 - protein, 722 aa, Homo sapiens
MLTFFLVSGGSLWLFVEFVLSLLEKMQTQEILRILRLPELGDLGQFFRSLSATTLVSMGALAAILAYWFTHRPKALQPPCNLLMQSEEVEDSGGARRSVIGSGPQLLTHYYDDARTMYQVFRRGLSISGNGPCLGFRKPKQPYQWLSYQEVADRAEFLGSGLLQHNCKACTDQFIGVFAQNRPEWIIVELACYTYSMVVVPLYDTLGPGAIRYIINTADISTVIVDKPQKAVLLLEHVERKETPGLKLIILMDPFEEALKERGQKCGVVIKSMQAVEDCGQENHQAPVPPQPDDLSIVCFTSGTTGNPKGAMLTHGNVVADFSGFLKVTEKVIFPRQDDVLISFLPLAHMFERVIQSVVYCHGGRVGFFQGDIRLLSDDMKALCPTIFPVVPRLLNRMYDKIFSQANTPLKRWLLEFAAKRKQAEVRSGIIRNDSIWDELFFNKIQASLGGCVRMIVTGAAPASPTVLGFLRAALGCQVYEGYGQTECTAGCTFTTPGDWTSGHVGAPLPCNHIKLVDVEELNYWACKGEGEICVRGPNVFKGYLKDPDRTKEALDSDGWLHTGDIGKWLPAGTLKIIDRKKHIFKLAQGEYVAPEKIENIYIRSQPVAQIYVHGDSLKAFLVGIVVPDPEVMPSWAQKRGIEGTYADLCTNKDLKKAILEDMVRLGKESGLHSFEQVKAIHIHSDMFSVQNGLLTPTLKAKRPELREYFKKQIEELYSISM

SEQ ID NO: 16 - DNA, 2166 bp, Homo sapiens
atgctgaccttcttcctcgtgtcggggggctccctctggctattcgtagagtttgtcctctcacttctggagaagatgcagacacaggagatcctgaggatactgcgactgcctgagctaggtgacttgggacagtttttccgcagcctctcggccaccaccctcgtgagtatgggtgccctggctgccatccttgcctactggttcactcaccggccaaaggccttgcagccgccatgcaacctcctgatgcagtcagaagaagtagaggacagtggcggggcacggcgatctgtgattgggtctggccctcagctacttacccactactatgatgatgcccggaccatgtaccaggtgttccgccgtgggcttagcatctcagggaatgggccctgtcttggtttcaggaagcctaagcagccttaccagtggctgtcctaccaggaggtggccgacagggctgaatttctggggtccggacttctccagcacaattgtaaagcatgcactgatcagtttattggtgtttttgcacaaaatcggccagagtggatcattgtggagctggcctgctacacatattccatggtggtggtcccgctctatgacaccctgggccctggggctatccgctacatcatcaatacagcggacatcagcaccgtgattgtggacaaacctcagaaggctgtgcttctgctagagcatgtggagaggaaggagactccaggcctcaagctgatcatcctcatggacccattcgaagaagccctgaaagagagagggcagaagtgcggggtggtcattaagtccatgcaggccgtggaggactgtggccaagagaatcaccaggctcctgtgcccccgcagcctgatgacctctccattgtgtgtttcacaagcggcacgacagggaacccaaaaggtgcgatgctcacccatgggaacgtggtggctgatttctcaggctttctgaaagtgacagagaaagtgatctttccgagacaggacgatgtgctcatctccttcctgcctctggctcacatgtttgagagagtaatccagtctgtcgtctattgccacggagggcgtgttggcttcttccagggagatatccgccttctctcagatgacatgaaggctctatgccccaccatcttccctgtggtcccacgactgctgaaccggatgtacgacaagatcttcagccaggcaaacacaccattaaagcgctggctcctggagtttgcagcaaagcgtaagcaagccgaggtccggagtggaatcatcaggaatgatagtatctgggatgaactcttctttaataagattcaggccagtcttggtgggtgtgtgcggatgattgttactggagcagccccagcatcaccaacagttctgggatttctccgggcagctctagggtgccaggtttatgaaggttatggccaaactgagtgcacagctggatgtaccttcaccactcctggcgactggacctcagggcacgtaggggcgccacttccctgcaatcatatcaagctcgttgatgttgaggaactgaactactgggcctgcaaaggagagggagagatatgtgtgagaggaccaaatgtgttcaaaggctacttgaaagatccagacaggacgaaggaggccctggacagcgatggctggcttcacactggagacatcggaaaatggctgccggcaggaactcttaaaattattgatcggaaaaagcatatatttaaacttgctcagggagaatatgttgcacccgagaagattgagaacatctacatccggagccaacctgtggcgcaaatctatgtccatggggacagcttaaaggcctttttggtaggcattgttgtgcctgaccctgaagttatgccctcctgggcccagaagagaggaattgaaggaacatatgcagatctctgcacaaataaggatctgaagaaagccattttggaagatatggtgaggttaggaaaagaaagtggactccattcttttgagcaggttaaagccattcacatccattctgacatgttctcagttcaaaatggcttgctgacaccaacactaaaagctaagagacctgagctgagagagtacttcaaaaaacaaatagaagagctttactcaatctccatg

SEQ ID NO: 17 - DNA, 21 bp, Artificial
  Primer
gtctcaccggcttcctcttct

SEQ ID NO: 18 - DNA, 35 bp, Artificial
  Primer
aaaggatccataatcctggatgtgctcatacaggc

(19) United States
(12) Patent Application Publication
(10) Pub. No.: US 2009/0155241 A1
(43) Pub. Date: Jun. 18, 2009

(54) Prophylactic/therapeutic agent for cancer

(75) Inventors: Tetsuo Mashima, Tokyo (JP); Takashi Tsuruo, Tokyo (JP)

Correspondence Address: FOLEY AND LARDNER LLP;SUITE 500, 3000 K STREET NW, WASHINGTON, DC, 20007, US

(73) Assignees: JAPANESE FOUNDATION FOR CANCER RESEARCH; TAKEDA PHARMACEUTICAL COMPANY LIMITED

(21) Appl. No.: 11/988,995

(22) PCT Filed: Jul. 22, 2005
(86) PCT No.: PCT/JP2005/013908

§ 371 Date: Jan. 18, 2008

(51) Int. Cl. A61K 39/395; C07H 21/02; C07K 16/18; G01N 33/53; C12Q 1/68; C12Q 1/00; A61K 31/7088; C12N 9/96; C07C 245/00

(52) U.S. Cl. 424/130.1; 536/24.5; 536/23.2; 530/387.9; 435/7.1; 435/6; 435/4; 514/44; 435/188; 534/550

(57) ABSTRACT

A compound or its salt that inhibits the activity of a protein having the same or substantially the same amino acid sequence as the amino acid sequence represented by SEQ ID NO: 1; a compound or its salt that inhibits the expression of a gene for the protein; an antisense polynucleotide comprising a nucleotide sequence complementary or substantially complementary to the nucleotide sequence of a polynucleotide encoding the protein or a partial peptide thereof or a part of the nucleotide sequence; an antibody against the protein; etc. are useful as preventive/therapeutic agents for cancer, apoptosis promoters for cancer cells, or the like.

### TECHNICAL FIELD

The present invention relates to prophylactic/therapeutic agents and diagnostic agents for cancer, screening of prophylactic/therapeutic agents for cancer, and the like.

### BACKGROUND ART

Apoptosis is a spontaneous cell death controlled on a molecular level and deeply involved in maintenance of homeostasis of living organisms (Strasser A., et al., Annu. Rev. Biochem., 69, 217-245, 2000). Since its molecular mechanism is important for negative control of cellular proliferation, it is suggested that the abnormality of apoptosis is deeply associated with oncogenesis. Intrinsic apoptotic execution pathway is induced primarily by release of cytochrome c from the mitochondria. The released cytochrome c binds to Apaf-1 protein, which triggers the formation of a macromolecular complex called apoptosome (Budihardjo, I. et al., Annu. Rev. Cell Dev. Biol., 15, 269-290, 1999). By caspase 9 activation, the apoptosome causes a series of protein degradation events to induce cell death. On the other hand, p53, which is a tumor suppressor gene product, plays an important role upstream of the apoptosis pathway initiated by release of cytochrome c from the mitochondria. p53 is a transcription factor and induces transcription of BAX, NOXA, PUMA, p53AIP1, etc. to activate apoptotic execution pathway initiated by release of cytochrome c from the mitochondria (Fridman, J. S. et al., Oncogene, 22, 9030-9040, 2003).

In cancer cells, it has been revealed that the activation and inactivation occur ambivalently in this apoptosis pathway. Activation of cancer gene products such as c-myc, Ras, etc. and increased proliferation accompanied thereby activate the intrinsic apoptosis pathway in cancer cells (Feamhead, H. O. et al., Proc. Natl. Acad. Sci. USA, 95, 13664-13669, 1998). For example, cell-cycle factor E2F-1 activated, accompanied by the increased proliferation, upregulates the expression of Apaf-1 or caspases, which are the factors involved in apoptosis pathway (Moroni, M. C., et al., Nat. Cell Biol., 3, 552-558, 2001). These reports suggest that intrinsic apoptotic execution pathway is potentially activated in cancer cells.

On the other hand, however, it is considered that activation of the apoptosis pathway by cancer selective environmental stresses such as expression of cancer gene products, hypoxic conditions, etc. becomes a selective pressure, resulting in further occurrence of some defect in the apoptosis pathway in the process of cancer development or malignant alteration (Johnstone, R. W. et al., Cell, 108, 153-164, 2002). The most important change is the gene mutation of p53, and this gene mutation is observed in about 50% of human cancer cells. The p53 defect in cancer cells is the main cause of resistance to various chemotherapeutic drugs and is a serious barrier to cancer therapy (E1-Deiry W. S. et al., Oncogene, 22, 7486-7495, 2003).

While it is known that abnormality of apoptotic molecular mechanism occurs widely in cancer cells and have a strong effect on chemosensitivity, defective pattern of apoptotic pathway in cancer cells is unknown. In particular, defective pattern of apoptosis pathway is unclear in p53-mutated cancer, which is almost incurable due to the resistance to chemotherapeutic drugs. Consequently, the target protein to induce cell death p53-mutated cancer-selectively is unclear and its inhibitor has not been developed.

Enzymes belonging to the acyl-CoA synthetase family (hereinafter referred to as ACS) are the enzyme synthesizing acyl-CoA which uses a long chain fatty acid (carbon number of 10 to 20) as a substrate. Since acyl-CoA is a substrate for intracellular synthesis of lipids such as phospholipids, etc. and fatty acid degradation/extension reactions, ACS plays a primary role in the intracellular metabolism of fatty acids. Since 1990, when the first ACS gene was cloned, five isozymes different in substrate selectivity or intracellular localization have been identified to date in human and rodent (Coleman, R. A., et al., J. Nutr., 132, 2123-2126, 2002). Among them, ACS4 which uses arachidonate as a selective substrate is overexpressed in human colon cancer (Cao, Y. et al., Cancer Res., 61, 8429-8434, 2001). Also, ACS5 localized in mitochondria is overexpression in human glioma (Yamashita, Y. et al., Oncogene, 19, 5919-5925, 2000).

1-Hydroxy-3-undeca-2,4,7-triene-1-ylidenetriaz-1-ene (Triacsin c) is known as the ACS inhibitor (Biochem. Biophys. Acta, 921, 595-598, 1987).

### DISCLOSURE OF INVENTION

Many human solid cancer cells are resistant to the existing anticancer drugs. Since most of the existing chemotherapeutic drugs work through the p53 function, p53-mutated cancer that is a widespread phenomenon occurred in 50% of human cancer cells becomes resistant to chemotherapeutic drugs, which makes treatment extremely difficult. A safe, excellent and novel agent for preventing/treating cancer, which improves resistance to chemotherapeutic drugs in cancer cells, especially p53-mutated cancer cells, has been earnestly desired.

In order to solve the foregoing problems, the present inventors have made extensive studies and as a result, clarified that p53 mutations and defects in apoptosis pathway occur complementarily in human cancer cells. In other words, the inventors have clarified that in cancer cells, especially in p53-mutated cancer, the activity of apoptosome located downstream of the pathway is retained and further upregulated as compared to normal cells. Furthermore, the inventors have made search for medicaments directly activating this upregulated apoptosis pathway and found ACS inhibitors. Based on these findings, the inventors have made further investigations and come to accomplish the present invention.

That is, the present invention provides the following features and so on.

[1] A preventive/therapeutic agent for cancer comprising a compound or a salt thereof which inhibits the activity of an enzyme belonging to the acyl-CoA synthetase family.

[2] The preventive/therapeutic agent according to [1] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[3] The preventive/therapeutic agent according to [1] above, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

[4] A preventive/therapeutic agent for cancer comprising a compound or a salt thereof which inhibits the gene expression of an enzyme belonging to the acyl-CoA synthetase family.

[5] The preventive/therapeutic agent according to [4] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO:7, SEQ ID NO:9, SEQ ID NO: 1, SEQ ID NO: 13 or SEQ ID NO: 15, its partial peptide, or a salt thereof.

[6] The preventive/therapeutic agent according to [4] above, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

[7] The preventive/therapeutic agent according to [1] through [6] above, wherein the cancer is p53-mutated cancer.

[8] A preventive/therapeutic agent for cancer, which comprises 1-hydroxy-3-undeca-2,4,7-triene-1-ylidentriaz-1-ene.

[9] An antisense polynucleotide comprising an entire or part of a nucleotide sequence complementary or substantially complementary to the nucleotide sequence of a polynucleotide encoding an enzyme belonging to the acyl-CoA synthetase family.

[10] The antisense polynucleotide according to [9] above, wherein the enzyme belonging to the acyl-CoA synthetase family is a protein comprising the same or substantially the same amino acid sequence as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[10a] The antisense polynucleotide according to [9] above, wherein the enzyme belonging to the acyl-CoA synthetase family is a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[11] A medicament comprising the antisense polynucleotide according to [9] above.

[12] A siRNA or shRNA to a polynucleotide encoding an enzyme belonging to the acyl-CoA synthetase family.

[13] The siRNA or shRNA according to [12] above, wherein the enzyme belonging to the acyl-CoA synthetase family is a protein comprising the same or substantially the same amino acid sequence as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[13a] The siRNA or shRNA according to [12] above, wherein the enzyme belonging to the acyl-CoA synthetase family is a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[14] A medicament comprising the siRNA or shRNA according to [12] above.

[15] The medicament according to [14] above, which is a preventive/therapeutic agent for cancer.

[16] A preventive/therapeutic agent for cancer comprising an antibody against an enzyme belonging to the acyl-CoA synthetase family.

[17] The preventive/therapeutic agent according to [16] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[17a] The preventive/therapeutic agent according to [16] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[18] A diagnostic agent for cancer comprising an antibody against an enzyme belonging to the acyl-CoA synthetase family.

[19] The diagnostic agent according to [18] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 13 SEQ ID NO:13 or SEQ ID NO: 15, its partial peptide, or a salt thereof.

[19a] The diagnostic agent according to [18] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[20] A diagnostic agent for cancer comprising a polynucleotide encoding an enzyme belonging to the acyl-CoA synthetase family.

[21] The diagnostic agent according to [20] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[21a] The diagnostic agent according to [20] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[22] A method for diagnosis of cancer, which comprises using an antibody against an enzyme belonging to the acyl-CoA synthetase family or a polynucleotide encoding an enzyme belonging to the acyl-CoA synthetase family.

[23] The method for diagnosis according to [22] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[23a] The method for diagnosis according to [22] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[24] Use of an enzyme belonging to the acyl-CoA synthetase family as a diagnostic marker of cancer.

[25] The use according to [24] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[25a] The use according to [24] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[26] A screening method of a medicament for preventing/treating cancer, which comprises using an enzyme belonging to the acyl-CoA synthetase family.

[26a] The screening method according to [26] above, wherein the medicament for preventing/treating cancer is a medicament that inhibits the activity of at least one acyl-CoA synthetase selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[26b] The screening method according to [26] above, wherein the medicament for preventing/treating cancer is a medicament that inhibits the activity of at least one acyl-CoA synthetase selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[27] The screening method according to [26] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[28] The screening method according to [26] above, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

[29] The screening method according to [26] above, wherein the cancer is p53-mutated cancer.

[30] A screening kit for a medicament for preventing/treating cancer, which comprises an enzyme belonging to the acyl-CoA synthetase family.

[31] The screening kit according to [30] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO:7, SEQ ID NO:9, SEQ ID NO:1, SEQ ID NO: 13 or SEQ ID NO: 15, its partial peptide, or a salt thereof.

[31a] The screening kit according to [30] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[32] A screening method for a medicament for preventing/treating cancer, which comprises using a polynucleotide encoding an enzyme belonging to the acyl-CoA synthetase family.

[32a] The screening method according to [32] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[32b] The screening method according to [32] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[33] A screening kit for a medicament for preventing/treating cancer, which comprises a polynucleotide encoding an enzyme belonging to the acyl-CoA synthetase family.

[33a] The screening kit according to [33] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[33b] The screening kit according to [33] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[34] A screening method of a medicament for preventing/treating cancer, which comprises measuring the amount or activity of an enzyme belonging to the acyl-CoA synthetase family.

[34a] The screening method according to [34] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[34b] The screening method according to [34] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[35] A method for preventing/treating cancer, which comprises inhibiting the activity of an enzyme belonging to the acyl-CoA synthetase family.

[36] The method for preventing/treating cancer according to [35] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[36a] The method for preventing/treating cancer according to [35] above,

wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[37] A method for preventing/treating cancer, which comprises inhibiting the gene expression of an enzyme belonging to the acyl-CoA synthetase family.

[38] The preventive/therapeutic method according to [37] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[38a] The preventive/therapeutic method according to [37] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[39] A method for preventing/treating cancer, which comprises administering to a mammal an effective dose of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family.

[39a] A method for preventing/treating cancer, which comprises administering to a mammal an effective dose of siRNA or shRNA to a polynucleotide encoding an enzyme belonging to the acyl-CoA synthetase family.

[40] Use of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family; to produce a preventive/therapeutic agent for cancer.

[40a] Use of siRNA or shRNA to a polynucleotide encoding an enzyme belonging to the acyl-CoA synthetase family; to produce a preventive/therapeutic agent for cancer.

[41] An agent for promoting the apoptosis of cancer cells or an agent for suppressing the growth of cancer cells, comprising a compound or a salt thereof which inhibits the activity of an enzyme belonging to the acyl-CoA synthetase family.

[41a] An agent for suppressing the metastasis/relapse of cancer, which comprises a compound or a salt thereof which inhibits the activity of an enzyme belonging to the acyl-CoA synthetase family.

[42] The agent according to [41] or [41a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO:7, SEQ ID NO: 9, SEQ ID NO: 11, SEQ ID NO:13, SEQ ID NO: 15, its partial peptide, or a salt thereof.

[43] The agent according to [41] or [41a] above, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

[44] An agent for promoting the apoptosis of cancer cells or an agent for suppressing the growth of cancer cells, which comprises a compound or a salt thereof which inhibits the gene expression of an enzyme belonging to the acyl-CoA synthetase family.

[44a] An agent for suppressing the metastasis/relapse of cancer, which comprises a compound or a salt thereof which inhibits the gene expression of an enzyme belonging to the acyl-CoA synthetase family.

[45] The agent according to [44] or [44a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO:7, SEQ ID NO: 9, SEQ ID NO: 11, SEQ ID NO:13 or SEQ ID NO: 15, its partial peptide, or a salt thereof.

[46] The agent according to [44] or [44a] above, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

[47] The agent according to [41] through [46] above, wherein the cancer is p53-mutated cancer.

[48] An agent for promoting the apoptosis of cancer cells or an agent for suppressing the growth of cancer cells, which comprises 1-hydroxy-3-undeca-2,4,7-triene-1-ylidenetriaz-1-ene.

[48a] An agent for suppressing the metastasis/relapse of cancer, which comprises 1-hydroxy-3-undeca-2,4,7-triene-1-ylidenetriazo-1-ene.

[49] The medicament according to [11] or [14] above, which is an agent for promoting the apoptosis of cancer cells or an agent for suppressing the growth of cancer cells.

[49a] The medicament according to [11] or [14] above, which is an agent for suppressing the metastasis/relapse of cancer.

[50] An agent for promoting the apoptosis of cancer cells or an agent for suppressing the growth of cancer cells, which comprises an antibody against an enzyme belonging to the acyl-CoA synthetase family.

[50a] An agent for suppressing the metastasis/relapse of cancer, which comprises an antibody against an enzyme belonging to the acyl-CoA synthetase family.

[51] The agent according to [50] or [50a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[51a] The agent according to [50] or [50a] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[52] A screening method of an agent for promoting the apoptosis of cancer cells or an agent for suppressing the growth of cancer cells, which comprises using an enzyme belonging to the acyl-CoA synthetase family.

[52a] A screening method for a medicament for suppressing the metastasis/relapse of cancer, which comprises using an enzyme belonging to the acyl-CoA synthetase family.

[53] The screening method according to [52] or [52a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[54] The screening method according to [52] or [52a] above, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

[55] The screening method according to [52] above, wherein the cancer is p53-mutated cancer.

[56] A screening kit for an agent for promoting the apoptosis of cancer cells or an agent for suppressing the growth of cancer cells, which comprises an enzyme belonging to the acyl-CoA synthetase family.

[56a] A screening kit for a medicament for suppressing the metastasis/relapse of cancer, which comprises an enzyme belonging to the acyl-CoA synthetase family.

[57] The screening kit according to [56] or [56a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[57a] The screening kit according to [56] or [56a] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[58] A screening method of an agent for promoting the apoptosis of cancer cells or an agent for suppressing the growth of cancer cells, which comprises using a polynucleotide encoding an enzyme belonging to the acyl-CoA synthetase family.

[58a] A screening method for a medicament for suppressing the metastasis/relapse of cancer, which comprises using a polynucleotide encoding an enzyme belonging to the acyl-CoA synthetase family.

[58b] The screening method according to [58] or [58a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[58c] The screening method according to [58] or [58a] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[59] A screening kit for a medicament for preventing/treating cancer, which comprises polynucleotide encoding enzyme belonging to the acyl-CoA synthetase family.

[59a] The screening kit according to [59] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[59b] The screening kit according to [59] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO:11, its partial peptide, or a salt thereof.

[60] A screening method of an agent for promoting the apoptosis of cancer cells or an agent for suppressing the growth of cancer cells, which comprises measuring the amount or activity of an enzyme belonging to the acyl-CoA synthetase family.

[60a] A screening method of a medicament for suppressing the metastasis/relapse of cancer, which comprises assaying the level or activity of an enzyme belonging to the acyl-CoA synthetase family.

[60b] The screening method according to [60] or [60a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[60c] The screening method according to [60] or [60a] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[61] A method for promoting the apoptosis of cancer cells or suppressing the growth of cancer cells, which comprises inhibiting the activity of an enzyme belonging to the acyl-CoA synthetase family.

[61a] A method for suppressing the metastasis/relapse of cancer, which comprises inhibiting the activity of an enzyme belonging to the acyl-CoA synthetase family.

[62] The method according to [61] or [61a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[62a] The method according to [61] or [61a] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[62b] The method according to [61] through [62a] above, wherein the cancer is p53-mutated cancer.

[63] A method for promoting the apoptosis of cancer cells or suppressing the growth of cancer cells, which comprises inhibiting the gene expression of an enzyme belonging to the acyl-CoA synthetase family.

[63a] A method for suppressing the metastasis/relapse of cancer, which comprises inhibiting the gene expression of an enzyme belonging to the acyl-CoA synthetase family.

[64] The method according to [63] or [63a] above, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

[64a] The method according to [63] or [63a] above, wherein the enzyme belonging to the acyl-CoA synthetase family is at least one selected from a protein comprising the same or substantially the same amino acid sequence as the amino acid sequence represented by SEQ ID NO: 5, SEQ ID NO: 7, SEQ ID NO: 9 or SEQ ID NO: 11, its partial peptide, or a salt thereof.

[64b] The method according to [63] through [64a] above, wherein the cancer is p53-mutated cancer.

[65] A method for promoting the apoptosis of cancer cells or suppressing the growth of cancer cells, which comprises administering to a mammal an effective dose of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family.

[65a] A method of suppressing the metastasis/relapse of cancer, which comprises administering to a mammal an effective dose of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family.

[65b] A method for promoting the apoptosis of cancer cells or suppressing the growth of cancer cells, which comprises administering to a mammal an effective dose of siRNA or shRNA to a polynucleotide encoding an enzyme belonging to the acyl-CoA synthetase family.

[65c] A method of suppressing the metastasis/relapse of cancer, which comprises administering to a mammal an effective dose of siRNA or shRNA to a polynucleotide encoding an enzyme belonging to the acyl-CoA synthetase family.

[65d] The method according to [65] through [65c] above, wherein the cancer is p53-mutated cancer.

[66] Use of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family; to produce an agent for promoting the apoptosis of cancer cells or an agent for suppressing the growth of cancer cells.

[66a] Use of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family; to produce an agent for suppressing the metastasis/relapse of cancer.

[66b] Use of siRNA or shRNA to a polynucleotide encoding an enzyme belonging to the acyl-CoA synthetase family; to produce an agent for promoting the apoptosis of cancer cells or an agent for suppressing the growth of cancer cells.

[66c] Use of siRNA or shRNA to a polynucleotide encoding an enzyme belonging to the acyl-CoA synthetase family; to produce an agent for suppressing the metastasis/relapse of cancer.

[66d] The method according to [66] through [66c] above, wherein the cancer is p53-mutated cancer.

[67] An anticancer drug resistance improving agent comprising a compound or a salt thereof which inhibits the activity of an enzyme belonging to the acyl-CoA synthetase family.

[67a] An agent for improving resistance to an anticancer drug, which comprises (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, (iv) an antisense polynucleotide comprising an entire or part of a nucleotide sequence complementary or substantially complementary to the nucleotide sequence of a polynucleotide encoding an enzyme belonging to the acyl-CoA synthetase family or (v) siRNA or shRNA to a polynucleotide encoding an enzyme belonging to the acyl-CoA synthetase family.

[68] The improving agent according to [67] above, wherein the anticancer drug resistance is p53 mutation-induced anticancer drug resistance.

[68a] The improving agent according to [67] above, wherein the anticancer drug is a chemotherapeutic agent.

[69] A method of improving anticancer drug resistance, which comprises inhibiting the activity of an enzyme belonging to the acyl-CoA synthetase family.

[70] A screening method of a medicament for improving anticancer drug resistance, which comprises using an enzyme belonging to the acyl-CoA synthetase family.

[70a] A screening method of a medicament for improving anticancer drug resistance, which comprises using a polynucleotide encoding an enzyme belonging to the acyl-CoA synthetase family.

[71] Use of an enzyme belonging to the acyl-CoA synthetase family as a diagnostic marker of anticancer drug sensitivity.

[71a] Use of a polynucleotide encoding an enzyme belonging to the acyl-CoA synthetase family as a diagnostic marker of anticancer drug sensitivity.

The “medicament for preventing/treating cancer” may be a substance per se (e.g., a synthetic compound, peptide, protein, antibody, non-peptide compound, fermentation product, cell extract, plant extract, animal tissue extract, blood plasma, etc.) having preventive/therapeutic effects on cancer, or may be a pharmaceutical preparation comprising said substance.

The “agent for promoting the apoptosis of cancer cells” may be a substance per se (e.g., a synthetic compound, peptide, protein, antibody, non-peptide compound, fermentation product, cell extract, plant extract, animal tissue extract, blood plasma, etc.) having effects of promoting the apoptosis of cancer cells, or may be a pharmaceutical preparation comprising said substance.

The “medicament for suppressing the growth of cancer cells may be a substance per se (e.g., a synthetic compound, peptide, protein, antibody, non-peptide compound, fermentation product, cell extract, plant extract, animal tissue extract, blood plasma, etc.) having effects of suppressing the growth of cancer cells, or may be a pharmaceutical preparation comprising said substance.

The “medicament for improving anticancer drug resistance” may be a substance per se (e.g., a synthetic compound, peptide, protein, antibody, non-peptide compound, fermentation product, cell extract, plant extract, animal tissue extract, blood plasma, etc.) having effects of improving anticancer drug resistance, or may be a pharmaceutical preparation comprising said substance.

The “medicament for suppressing the metastasis/relapse of cancer” may be a substance per se (e.g., a synthetic compound, peptide, protein, antibody, non-peptide compound, fermentation product, cell extract, plant extract, animal tissue extract, blood plasma, etc.) having suppressing effects on the metastasis/relapse of cancer, or may be a pharmaceutical preparation comprising said substance.

### BRIEF DESCRIPTION OF THE DRAWINGS

FIG. 1A shows the distribution of p53 gene mutations and apoptosome activities in human solid cancer.

FIG. 1B shows the apoptosome activities in human normal tissue-derived cells, human normal cell lines and p53-mutated cancer cell lines.

FIG. 2 shows the correlation between the apoptosome activity and Triacsin c sensitivity (-LOG (GI₅₀)) of cancer cells.

FIG. 3A shows the cell growth inhibitory effects on p53-mutated cancer cells and normal cell lines by Triacsin c.

FIG. 3B shows the assay results of caspase activation by Triacsin c.

FIG. 4A shows the results of expression analysis of ACS5 protein.

FIG. 4B shows the inhibitory effects on ACS activity by Triacsin c.

FIG. 4C shows the cell growth suppressing effects by Triacsin c.

FIG. 4D shows the assay results of caspase activation by Triacsin c.

FIG. 5 shows the growth suppressing effects on p53-mutated cancer by Triacsin c.

FIG. 6A shows the cell viability after etoposide and 5-fluorouracil treatments.

FIG. 6B shows the cell viability when treated with etoposide alone and when treated with etoposide and Triacsin c in combination.

### BEST MODE FOR CARRYING OUT THE INVENTION

The enzyme belonging to the acyl-CoA synthetase family (hereinafter sometimes referred to as the protein of the present invention or the protein used in the present invention) may be any protein derived from any cells of human and other warm-blooded animals (e.g. guinea pig, rat, mouse, fowl, rabbit, swine, sheep, bovine, monkey, etc.) (e.g., retina cells, hepatocytes, splenocytes, nerve cells, glial cells, β cells of pancreas, bone marrow cells, mesangial cells, Langerhans' cells, epidermic cells, epithelial cells, endothelial cells, fibroblasts, fibrocytes, myocytes, fat cells, immune cells (e.g., macrophage, T cells, B cells, natural killer cells, mast cells, neutrophils, basophils, eosinophils, monocytes, platelets, etc.), megakaryocyte, synovial cells, chondrocytes, bone cells, osteoblasts, osteoclasts, mammary gland cells, hepatocytes or interstitial cells, the corresponding precursor cells, stem cells, cancer cells, etc.), or any tissues where such cells are present, e.g., brain or any region of the brain (e.g., retina, olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, muscle, lung, gastrointestinal tract (e.g., large intestine and small intestine), blood vessel, heart, thymus, spleen, submandibular gland, peripheral blood, prostate, testis, ovary, placenta, uterus, bone, joint, skeletal muscle, etc.; proteins derived from hemocyte type cells or their cultured cells (e.g., MEL, M1, CTLL-2, HT-2, WEHI-3, HL-60, JOSK-1, K562, ML-1, MOLT-3, MOLT-4, MOLT-10, CCRF-CEM, TALL-1, Jurkat, CCRT-HSB-2, KE-37, SKW-3, HUT-78, HUT-102, H9, U937, THP-1, HEL, JK-1, CMK, KO-812, MEG-01, etc.); the protein may also be a synthetic protein.

The enzyme belonging to the acyl-CoA synthetase family includes at least one selected from the protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

The amino acid sequence which is substantially the same as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 includes amino acid sequences having at least about 50% homology, preferably at least about 60% homology, more preferably at least about 70% homology, still more preferably at least about 80% homology, much more preferably at least about 90% homology and most preferably at least about 95% homology, to the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15; and the like.

Homology of the amino acid sequences can be measured under the following conditions (an expectation value=10; gaps are allowed; matrix=BLOSUM62; filtering ═OFF) using a homology scoring algorithm NCBI BLAST (National Center for Biotechnology Information Basic Local Alignment Search Tool).

Preferred examples of the protein comprising substantially the same amino acid sequence as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 are proteins comprising substantially the same amino acid sequence as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 and having an activity substantially equivalent to that of the protein comprising the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, etc.

As the substantially equivalent activity, there are, for example, an acyl-CoA synthetase activity, and the like. The term substantially equivalent is used to mean that these properties are equivalent in terms of their nature (e.g., physiologically or pharmacologically). It is thus preferred that acyl-CoA synthetases, etc. are equivalent (e.g., about 0.01 to 100 times, preferably about 0.1 to 10 times, more preferably 0.5 to 2 times), but differences in degree such as a level of these activities, quantitative factors such as a molecular weight of the protein may be present and allowable.

The acyl-CoA synthetase can be assayed by publicly known methods described in, for example, J. Biol. Chem., 256, 5702-5707, 1981, or their modifications. Specifically, the protein of the present invention is reacted in 0.5 ml of a solution containing 0.2 mM Tris-HCl buffer (pH7.5), 2.5 mM ATP, 8 mM MgCl₂, 2 mM EDTA, 20 mM NaF, 0.1% (w/v) Triton X-100, 10 ∥M [1-¹⁴C] palmitate (5 μCi/μmol) and 0.5 mM coenzyme A (CoA) at 35° C. for 10 minutes. The reaction is initiated by adding CoA and stopped by adding 2.5 ml of isopropanol:n-heptane:1M sulfuric acid (40:10:1, v/v). After the reaction is stopped, 0.5 ml of water and 2.5 ml of n-heptane are added to remove the organic solvent layer containing the unreacted fatty acids. The aqueous layer is further washed 3 times with 2.5 ml of n-heptane and the radioactivity retained in the aqueous layer is counted with a scintillation counter.

The protein of the present invention includes so-called muteins such as proteins comprising (i) the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, of which at least 1 or 2 (e.g., about 1 to about 100, preferably about 1 to about 30, more preferably about 1 to about 10 and most preferably several (1 to 5)) amino acids are deleted; (ii) the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, to which at least 1 or 2 (e.g., about 1 to about 100, preferably about 1 to about 30, more preferably about 1 to about 10 and most preferably several (1 to 5)) amino acids are added; (iii) the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, in which at least 1 or 2 (e.g., about 1 to about 100, preferably about 1 to about 30, more preferably about 1 to about 10 and most preferably several (1 to 5)) amino acids are inserted; (iv) the amino acid sequence represented by SEQ ID NO: 1, SEQ ID NO: 3, SEQ ID NO: 5, SEQ ID NO: 7, SEQ ID NO:9, SEQ ID NO: 11, SEQ ID NO: 13 or SEQ ID NO: 15, in which at least 1 or 2 (e.g., about 1 to about 100, preferably about 1 to about 30, more preferably about 1 to about 10 and most preferably several (1 to 5)) amino acids are substituted by other amino acids; or (v) a combination of these amino acid sequences; and the like.

Where the amino acid sequence is inserted, deleted or substituted as described above, the position of its insertion, deletion or substitution is not particularly limited.

As used herein, the proteins are represented in a conventional way of describing peptides, that is, the N-terminus (amino end) at the left hand and the C-terminus (carboxyl end) at the right hand. In the protein used in the present invention including the protein comprising the amino acid sequence represented by SEQ ID NO: 1, the C-terminus may be in any form of a carboxyl group (—COOH), a carboxylate (—COO⁻), an amide (—CONH₂) and an ester (—COOR).

Herein, examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl such as a phenyl-C₁₋₂ alkyl group, e.g., benzyl, phenethyl, etc.; an α-naphthyl-C₁₋₂ alkyl group such as α-naphthylmethyl, etc.; pivaloyloxymethyl and the like.

Where the protein used in the present invention contains a carboxyl group (or a carboxylate) at a position other than the C-terminus, the carboxyl group may be amidated or esterified and such an amide or ester is also included within the protein used in the present invention. Examples of the ester group in this case are the C-terminal esters described above, etc.

Furthermore, examples of the protein used in the present invention include variants wherein the amino group at the N-terminal amino acid residues (e.g., methionine residue) is protected with a protecting group (e.g., a C₁₋₆ acyl group such as a C₁₋₆ alkanoyl group, e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₁₋₆ alkanoyl group, e.g., formyl group, acetyl group, etc.), or conjugated proteins such as glycoproteins having sugar chains; etc.

Specific examples of the protein used in the present invention include a protein comprising the amino acid sequence represented by SEQ ID NO: 1, a protein comprising the amino acid sequence represented by SEQ ID NO: 3, a protein comprising the amino acid sequence represented by SEQ ID NO: 5, a protein comprising the amino acid sequence represented by SEQ ID NO: 7, a protein comprising the amino acid sequence represented by SEQ ID NO: 9, a protein comprising the amino acid sequence represented by SEQ ID NO: 11, a protein comprising the amino acid sequence represented by SEQ ID NO: 13, a protein comprising the amino acid sequence represented by SEQ ID NO: 15, and the like.

The partial peptide of the protein used in the present invention may be any peptide as long as it is a partial peptide of the protein used in the present invention described above and preferably has similar properties to those of the protein used in the present invention described above.

For example, there are used peptides containing, e.g., at least 20, preferably at least 50, more preferably at least 70, much more preferably at least 100 and most preferably at least 200 amino acids, in the constituent amino acid sequence of the protein used in the present invention, etc.

The partial peptide used in the present invention may be peptides comprising the amino acid sequence, of which at least 1 or 2 (preferably about 1 to about 20, more preferably about 1 to about 10 and most preferably several (1 to 5)) amino acids may be deleted; peptides, to which at least 1 or 2 (preferably about 1 to about 20, more preferably about 1 to about 10 and most preferably several (1 to 5)) amino acids may be added; peptides, in which at least 1 or 2 (preferably about 1 to about 20, more preferably about 1 to about 10 and most preferably several (1 to 5)) amino acids may be inserted; or peptides, in which at least 1 or 2 (preferably about 1 to about 20, more preferably several and most preferably about 1 to about 5) amino acids may be substituted by other amino acids.

In the partial peptide used in the present invention, the C-terminus may be in any form of a carboxyl group (—COOH), a carboxylate (—COO⁻), an amide (—CONH₂) or an ester (—COOR).

Furthermore, the partial peptide used in the present invention includes variants having a carboxyl group (or a carboxylate) at a position other than the C-terminus, those wherein the amino group at the N-terminal amino acid residues (e.g., methionine residue) is protected with a protecting group; those wherein the N-terminal region is cleaved in vivo and the glutamyl group thus formed is pyroglutaminated; those wherein a substituent on the side chain of an amino acid in the molecule is protected with a suitable protecting group, or conjugated peptides such as so-called glycopeptides having sugar chains; etc., as in the protein used in the present invention described above.

The partial peptide used in the present invention may also be used as an antigen for producing antibodies.

As salts of the protein or partial peptides used in the present invention, salts with physiologically acceptable acids (e.g., inorganic acids or organic acids) or bases (e.g., alkali metal salts) may be employed, preferably in the form of physiologically acceptable acid addition salts. Examples of such salts include salts with inorganic acids (e.g., hydrochloric acid, phosphoric acid, hydrobromic acid and sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid and benzenesulfonic acid), and the like.

The protein or its partial peptide used in the present invention or salts thereof may be manufactured by publicly known methods used to purify a protein from human or warm-blooded animal cells or tissues described above. Alternatively, they may also be manufactured by culturing transformants containing DNAs encoding these proteins. They may also be manufactured by a modification of the methods for peptide synthesis, which will be later described.

Where these proteins are produced from human or mammalian tissues or cells, human or mammalian tissues or cells are homogenized, extracted with an acid or the like, and the extract is purified and isolated by a combination of chromatography techniques such as reverse phase chromatography, ion exchange chromatography, and the like.

To synthesize the protein or partial peptide used in the present invention or its salts, or amides thereof, commercially available resins that are used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenyl acetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl)phenoxy resin, etc. Using these resins, amino acids, in which α-amino groups and functional groups on the side chains are appropriately protected, are condensed on the resin in accordance with the sequence of the objective protein according to various condensation methods publicly known in the art. At the end of the reaction, the protein or partial peptide is excised from the resin and at the same time, the protecting groups are removed. Then, intramolecular disulfide bond-forming reaction is performed in a highly diluted solution to obtain the objective protein or partial peptide, or amides thereof.

For condensation of the protected amino acids described above, a variety of activation reagents for protein synthesis may be used, and carbodiimides are particularly employed. Examples of such carbodiimides include DCC, N,N′-diisopropylcarbodiimide, N-ethyl-N′-(3-dimethylaminopropyl)carbodiimide, etc. For activation by these reagents, the protected amino acids in combination with a racemization inhibitor (e.g., HOBt, HOOBt) are added directly to the resin, or the protected amino acids are previously activated in the form of symmetric acid anhydrides, HOBt esters or HOOBt esters, followed by adding the thus activated protected amino acids to the resin.

Solvents suitable for use to activate the protected amino acids or condense with the resin may be appropriately chosen from solvents that are known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitriles such as acetonitrile, propionitrile, etc.; esters such as methyl acetate, ethyl acetate, etc.; and appropriate mixtures of these solvents. The reaction temperature is appropriately chosen from the range known to be applicable to protein binding reactions and is usually selected in the range of approximately −20° C. to 50° C. The activated amino acid derivatives are used generally in an excess of 1.5 to 4 times. The condensation is examined using the ninhydrin reaction; when the condensation is insufficient, the condensation can be completed by repeating the condensation reaction without removal of the protecting groups. When the condensation is yet insufficient even after repeating the reaction, unreacted amino acids are acetylated with acetic anhydride or acetylimidazole to avoid any possible effect on the subsequent reaction.

Examples of the protecting groups used to protect the starting amino groups include Z, Boc, t-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (linear, branched or cyclic alkyl esterification of, e.g., methyl, ethyl, propyl, butyl, t-butyl, cyclopentyl, cyclohexyl, cycloheptyl, cyclooctyl, 2-adamantyl, etc.), aralkyl esterification (e.g., benzyl ester, 4-nitrobenzyl ester, 4-methoxybenzyl ester, 4-chlorobenzyl ester, benzhydryl ester, etc.), phenacyl esterification, benzyloxycarbonyl hydrazidation, t-butoxycarbonyl hydrazidation, trityl hydrazidation, or the like.

The hydroxyl group of serine can be protected through, for example, its esterification or etherification. Examples of groups appropriately used for the esterification include a lower (C₁₋₆) alkanoyl group, such as acetyl group, an aroyl group such as benzoyl group, and a group derived from carbonic acid such as benzyloxycarbonyl group, ethoxycarbonyl group, etc. Examples of a group appropriately used for the etherification include benzyl group, tetrahydropyranyl group, t-butyl group, etc.

Examples of groups for protecting the phenolic hydroxyl group of tyrosine include Bzl, Cl₂-Bzl, 2-nitrobenzyl, Br-Z, t-butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting material include the corresponding acid anhydrides, azides, activated esters [esters with alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)], etc. As the amino acids in which the amino groups are activated in the starting material, the corresponding phosphoric amides are employed.

To eliminate (split off) the protecting groups, there are used catalytic reduction under hydrogen gas flow in the presence of a catalyst such as Pd-black or Pd-carbon; an acid treatment with anhydrous hydrogen fluoride, methanesulfonic acid, trifluoromethanesulfonic acid, trifluoroacetic acid, or a mixture solution of these acids; a treatment with a base such as diisopropylethylamine, triethylamine, piperidine or piperazine; reduction with sodium in liquid ammonia, etc. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol, 1,2-ethanedithiol, etc. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol, 1,4-butanedithiol, etc. as well as by a treatment with an alkali such as a dilute sodium hydroxide solution, dilute ammonia, etc.

Protection of functional groups that should not be involved in the reaction of the starting materials, protecting groups, elimination of the protecting groups and activation of functional groups involved in the reaction may be appropriately selected from publicly known groups and publicly known means.

In another method for obtaining the amides of the desired protein or partial peptide, for example, the α-carboxyl group of the carboxy terminal amino acid is first protected by amidation; the peptide (protein) chain is then extended from the amino group side to a desired length. Subsequently, a protein or partial peptide, in which only the protecting group of the N-terminal α-amino group of the peptide chain has been eliminated, and a protein or partial peptide, in which only the protecting group of the C-terminal carboxyl group has been eliminated, are manufactured. The two proteins or peptides are condensed in a mixture of the solvents described above. The details of the condensation reaction are the same as described above. After the protected protein or peptide obtained by the condensation is purified, all the protecting groups are eliminated by the method described above to give the desired crude protein or peptide. This crude protein or peptide is purified by various known purification means. Lyophilization of the major fraction gives the amide of the desired protein or peptide.

To prepare the esterified protein or peptide, for example, the α-carboxyl group of the carboxy terminal amino acid is condensed with a desired alcohol to prepare the amino acid ester, which is followed by procedures similar to the preparation of the amidated protein or peptide above to give the desired esterified protein or peptide.

The partial peptide used in the present invention or salts thereof can be manufactured by publicly known methods for peptide synthesis, or by cleaving the protein used in the present invention with an appropriate peptidase. For the methods for peptide synthesis, for example, either solid phase synthesis or liquid phase synthesis may be used. That is, the partial peptide or amino acids that can construct the partial peptide used in the present invention are condensed with the remaining part. Where the product contains protecting groups, these protecting groups are removed to give the desired peptide. Publicly known methods for condensation and elimination of the protecting groups are described in (i) to (v) below.

### (i) M. Bodanszky & M. A. Ondetti: Peptide Synthesis, Interscience Publishers, New York (1966)

### (ii) Schroeder & Luebke: The Peptide, Academic Press, New York (1965)

(iii) Nobuo Izumiya, et al.: Peptide Gosei-no-Kiso to Jikken (Basics and experiments of peptide synthesis), published by Maruzen Co. (1975)

### (iv) Haruaki Yajima & Shunpei Sakakibara: Seikagaku Jikken Koza (Biochemical Experiment) 1, Tanpakushitsu no Kagaku (Chemistry of Proteins) IV, 205 (1977)

(v) Haruaki Yajima, ed.: Zoku lyakuhin no Kaihatsu (A sequel to Development of Pharmaceuticals), Vol. 14, Peptide Synthesis, published by Hirokawa Shoten

After completion of the reaction, the partial peptide used in the present invention may be purified and isolated by a combination of conventional purification methods such as solvent extraction, distillation, column chromatography, liquid chromatography and recrystallization. When the partial peptide obtained by the above methods is in a free form, the partial peptide can be converted into an appropriate salt by a publicly known method or its modification; conversely when the partial peptide is obtained in a salt form, it can be converted into a free form or other different salt form by a publicly known method or its modification.

The polynucleotide encoding the protein used in the present invention may be any substance so long as it contains the nucleotide sequence encoding the protein used in the present invention described above. Preferably, the polynucleotide is a DNA. The DNA may also be any one of genomic DNA, genomic DNA library, cDNA derived from the cells or tissues described above, cDNA library derived from the cells or tissues described above and synthetic DNA.

The vector used for the library may be any of bacteriophage, plasmid, cosmid, phagemid and the like. In addition, the DNA can be amplified by reverse transcriptase polymerase chain reaction (hereinafter abbreviated as RT-PCR) with total RNA or mRNA fraction prepared from the above-described cells or tissues.

Examples of the DNA encoding the protein used in the present invention may be a DNA comprising the nucleotide sequence represented by SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, or a DNA comprising a nucleotide sequence hybridizable to the nucleotide sequence represented by SEQ ID NO: 2, SEQ ID NO: 4, SEQ ID NO:6, SEQ ID NO: 8, SEQ ID NO: 10, SEQ ID NO: 12, SEQ ID NO: 14 or SEQ ID NO: 16 under high stringent conditions and encoding a protein which has substantially the same properties as those of the protein comprising the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15.

As the DNA hybridizable to the nucleotide sequence represented by SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 under high stringent conditions, there are employed, for example, DNAs comprising nucleotide sequences having at least about 50% homology, preferably at least about 60% homology, preferably at least about 70% homology, preferably at least about 80% homology, preferably at least about 90% homology and preferably at least about 95% homology, to the nucleotide sequence represented by SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16; and the like.

Homology in the nucleotide sequence can be measured under the following conditions (an expectation value=10; gaps are allowed; filtering ═ON; match score=1; mismatch score=−3) using the homology scoring algorithm NCBI BLAST (National Center for Biotechnology Information Basic Local Alignment Search Tool).

Hybridization can be carried out by publicly known methods or modifications thereof, for example, by the method described in Molecular Cloning, 2nd ed. (J. Sambrook et al., Cold Spring Harbor Lab. Press, 1989). A commercially available library can also be used according to the instructions in the manufacturer's protocol attached. More preferably, the hybridization can be carried out preferably under high stringent conditions.

The high stringent conditions used herein are, for example, those in a sodium concentration at about 19 to 40 mM, preferably about 19 to 20 mM at a temperature of about 50 to 70° C., preferably about 60 to 65° C. In particular, hybridization conditions in a sodium concentration at about 19 mM at a temperature of about 65° C. are most preferred.

More specifically, there are employed a DNA comprising the nucleotide sequence represented by SEQ ID NO: 2, etc. as the DNA encoding the protein comprising the amino acid sequence represented by SEQ ID NO: 1; a DNA comprising the nucleotide sequence represented by SEQ ID NO: 4, etc. as the DNA encoding the protein comprising the amino acid sequence represented by SEQ ID NO: 3; a DNA comprising the nucleotide sequence represented by SEQ ID NO: 6, etc. as the DNA encoding the protein comprising the amino acid sequence represented by SEQ ID NO: 5; a DNA comprising the nucleotide sequence represented by SEQ ID NO: 8, etc. as the DNA encoding the protein comprising the amino acid sequence represented by SEQ ID NO: 7; a DNA comprising the nucleotide sequence represented by SEQ ID NO: 10, etc. as the DNA encoding the protein comprising the amino acid sequence represented by SEQ ID NO: 9; a DNA comprising the nucleotide sequence represented by SEQ ID NO: 12, etc. as the DNA encoding the protein comprising the amino acid sequence represented by SEQ ID NO: 11; a DNA comprising the nucleotide sequence represented by SEQ ID NO: 14, etc. as the DNA encoding the protein comprising the amino acid sequence represented by SEQ ID NO: 13; and a DNA comprising the nucleotide sequence represented by SEQ ID NO: 16, etc. as the DNA encoding the protein comprising the amino acid sequence represented by SEQ ID NO: 15.

The DNA encoding the partial peptide used in the present invention may be any DNA so long as it contains the nucleotide sequence encoding the partial peptide used in the present invention described above. The DNA may also be any of genomic DNA, genomic DNA library, cDNA derived from the cells and tissues described above, cDNA library derived from the cells and tissues described above and synthetic DNA.

As the DNA encoding the partial peptide used in the present invention, there are employed, for example, a DNA comprising a part of the DNA having the nucleotide sequence represented by SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, or a DNA comprising a nucleotide sequence hybridizable to the nucleotide sequence represented by SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 under high stringent conditions and comprising a part of DNA encoding a protein having the activities of substantially the same nature as those of the protein of the present invention, and the like.

The DNA hybridizable to the nucleotide sequence represented by SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 has the same significance as described above.

Methods for the hybridization and the high stringent conditions which can be used are the same as those described above.

For cloning of DNAs that completely encode the protein or partial peptide used in the present invention (hereinafter sometimes merely referred to as the protein of the present invention in the description of cloning of DNAs encoding the same and their expression), the DNA can be either amplified by PCR using synthetic DNA primers containing a part of the nucleotide sequence encoding the protein of the present invention, or the DNA inserted into an appropriate vector can be selected by hybridization with a labeled DNA fragment or synthetic DNA that encodes a part or entire region of the protein of the present invention. The hybridization can be carried out, for example, according to the method described in Molecular Cloning, 2nd (J. Sambrook et al., Cold Spring Harbor Lab. Press, 1989). Where the hybridization is carried out using commercially available library, the procedures may be conducted in accordance with the protocol described in the instructions attached.

Conversion of the nucleotide sequence of DNA can be effected by publicly known methods such as the ODA-LA PCR method, the Gapped duplex method, the Kunkel method, etc., or its modification, using a publicly known kit, e.g., Mutan™-super Express Km (Takara Shuzo Co., Ltd.) or Mutan™-K (Takara Shuzo Co., Ltd.), etc.

The cloned DNA encoding the protein can be used as it is, depending upon purpose or, if desired, after digestion with a restriction enzyme or after addition of a linker thereto. The DNA may contain ATG as a translation initiation codon at the 5′ end thereof and TAA, TGA or TAG as a translation termination codon at the 3′ end thereof. These translation initiation and termination codons may also be added by using an appropriate synthetic DNA adapter.

The expression vector for the protein of the present invention can be manufactured, for example, by (i) excising the desired DNA fragment from the DNA encoding the protein of the present invention, and then (ii) ligating the DNA fragment with an appropriate expression vector downstream of a promoter in the expression vector.

Examples of the vector include plasmids derived form E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNA I/Neo, etc.

The promoter used in the present invention may be any promoter if it matches well with a host to be used for gene expression. In the case of using animal cells as the host, examples of the promoter include SRα promoter, SV40 promoter, LTR promoter, CMV promoter, HSV-TK promoter, etc.

Among them, it is preferred to use CMV (cytomegalovirus) promoter, SRα promoter, etc. Where the host is bacteria of the genus Escherichia, preferred examples of the promoter include trp promoter, lac promoter, recA promoter, XPL promoter, 1pp promoter, T7 promoter, etc. In the case of using bacteria of the genus Bacillus as the host, preferred example of the promoter are SPO1 promoter, SPO2 promoter, penP promoter, etc. When yeast is used as the host, preferred examples of the promoter are PHO5 promoter, PGK promoter, GAP promoter, ADH promoter, etc. When insect cells are used as the host, preferred examples of the promoter include polyhedrin prompter, P10 promoter, etc.

In addition to the foregoing examples, the expression vector may further optionally contain an enhancer, a splicing signal, a poly A addition signal, a selection marker, SV40 replication origin (hereinafter sometimes abbreviated as SV40ori), etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. In particular, when dhfr gene is used as the selection marker using dhfr gene-deficient Chinese hamster cells, selection can also be made on a thymidine free medium.

If necessary, a signal sequence that matches with a host is added to the N-terminus of the protein of the present invention. Examples of the signal sequence that can be used are PhoA signal sequence, OmpA signal sequence, etc. when bacteria of the genus Escherichia is used as the host; α-amylase signal sequence, subtilisin signal sequence, etc. when bacteria of the genus Bacillus is used as the host; MFα signal sequence, SUC2 signal sequence, etc. when yeast is used as the host; and insulin signal sequence, α-interferon signal sequence, antibody molecule signal sequence, etc. when animal cells are used as the host, respectively.

Using the vector containing the DNA encoding the protein of the present invention thus constructed, transformants can be manufactured.

Examples of the host, which may be employed, are bacteria belonging to the genus Escherichia, bacteria belonging to the genus Bacillus, yeast, insect cells, insects, animal cells, etc.

Specific examples of the bacteria belonging to the genus Escherichia include Escherichia coli K12 DH1 [Proc. Natl. Acad. Sci. U.S.A., 60, 160 (1968)], JM103 [Nucleic Acids Research, 9, 309 (1981)], JA221 [Journal of Molecular Biology, 120, 517 (1978)], HB101 [Journal of Molecular Biology, 41, 459 (1969)], C600 [Genetics, 39, 440 (1954)], etc.

Examples of the bacteria belonging to the genus Bacillus include Bacillus subtilis MI114 [Gene, 24, 255 (1983)], 207-21 [Journal of Biochemistry, 95, 87 (1984)], etc.

Examples of yeast include Saccharomyces cereviseae AH22, AH22R⁻, NA87-11A, DKD-5D, 20B-12, Schizosaccharomyces pombe NCYC1913, NCYC2036, Pichia pastoris KM71, etc.

Examples of insect cells include, for the virus AcNPV, Spodoptera frugiperda cell (Sf cell), MG1 cell derived from mid-intestine of Trichoplusia ni, High Five™ cell derived from egg of Trichoplusia ni, cells derived from Mamestra brassicae, cells derived from Estigmena acrea, etc.; and for the virus BmNPV, Bombyx mori N cell (BmN cell), etc. is used. Examples of the Sf cell which can be used are Sf9 cell (ATCC CRL1711), Sf21 cell (both cells are described in Vaughn, J. L. et al., In Vivo, 13, 213-217 (1977)), etc.

As the insect, for example, a larva of Bombyx mori can be used [Maeda et al., Nature, 315, 592 (1985)].

Examples of animal cells include simian cell COS-7, Vero, Chinese hamster cell CHO (hereinafter referred to as CHO cells), dhfr gene-deficient Chinese hamster cell CHO (hereinafter simply referred to as CHO (dhfr⁻) cells), mouse L cells, mouse AtT-20, mouse myeloma cells, mouse ATDC5 cells, rat GH3, human FL cells, etc.

Bacteria belonging to the genus Escherichia can be transformed, for example, by the method described in Proc. Natl. Acad. Sci. U.S.A., 69, 2110 (1972), Gene, 17, 107 (1982), etc.

Bacteria belonging to the genus Bacillus can be transformed, for example, by the method described in Molecular & General Genetics, 168, 111 (1979), etc.

Yeast can be transformed, for example, by the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55 (1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995) (published by Shujunsha), or Virology, 52, 456 (1973).

Thus, the transformants transformed with the expression vectors bearing the DNAs encoding the protein can be obtained.

Where the host is bacteria belonging to the genus Escherichia or the genus Bacillus, the transformant can be appropriately cultured in a liquid medium which contains materials required for growth of the transformant such as carbon sources, nitrogen sources, inorganic materials, and the like. Examples of the carbon sources include glucose, dextrin, soluble starch, sucrose, etc.; examples of the nitrogen sources include inorganic or organic materials such as ammonium salts, nitrate salts, corn steep liquor, peptone, casein, meat extract, soybean cake, potato extract, etc.; and, examples of the inorganic materials are calcium chloride, sodium dihydrogenphosphate, magnesium chloride, etc. In addition, yeast extracts, vitamins, growth promoting factors etc. may also be added to the medium. Preferably, pH of the medium is adjusted to about 5 to about 8.

A preferred example of the medium for culturing the bacteria belonging to the genus Escherichia is M9 medium supplemented with glucose and Casamino acids [Miller, Journal of Experiments in Molecular Genetics, 431-433, Cold Spring Harbor Laboratory, New York, 1972]. If necessary, a chemical such as 3β-indolylacrylic acid can be added to the medium thereby to activate the promoter efficiently.

Where the bacteria belonging to the genus Escherichia are used as the host, the transformant is usually cultivated at about 15 to 43° C. for about 3 to 24 hours. If necessary, the culture may be aerated or agitated.

Where the bacteria belonging to the genus Bacillus are used as the host, the transformant is cultured generally at about 30 to 40° C. for about 6 to 24 hours. If necessary, the culture can be aerated or agitated.

Where yeast is used as the host, the transformant is cultivated, for example, in Burkholder's minimal medium [Bostian, K. L. et al., Proc. Natl. Acad. Sci. U.S.A., 77, 4505 (1980)] or in SD medium supplemented with 0.5% Casamino acids [Bitter, G. A. et al., Proc. Natl. Acad. Sci. U.S.A., 81, 5330 (1984)]. Preferably, pH of the medium is adjusted to about 5 to 8. In general, the transformant is cultivated at about 20 to 35° C. for about 24 to 72 hours. If necessary, the culture can be aerated or agitated.

Where insect cells or insects are used as the host, the transformant is cultivated in, for example, Grace's Insect Medium (Grace T. C. C., Nature, 195, 788 (1962)) to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary, the culture can be aerated or agitated.

Where animal cells are employed as the host, the transformant is cultured in, for example, MEM medium containing about 5 to 20% fetal bovine serum [Science, 122, 501 (1952)], DMEM medium [Virology, 8, 396 (1959)], RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultivated at about 30° C. to about 40° C. for about 15 to 60 hours and, if necessary, the culture can be aerated or agitated.

As described above, the protein of the present invention can be produced in the transformant, on the cell membrane of the transformant, or outside of the transformant.

The protein of the present invention can be separated and purified from the culture described above by the following procedures.

When the protein of the present invention is extracted from the bacteria or cells, the bacteria or cell is collected after culturing by a publicly known method and suspended in an appropriate buffer. The bacteria or cell is then disrupted by publicly known methods such as ultrasonication, a treatment with lysozyme and/or freeze-thaw cycling, followed by centrifugation, filtration, etc to produce crude extract of the protein. Thus, the crude extract of the protein can be obtained. The buffer used for the procedures may contain a protein denaturant such as urea or guanidine hydrochloride, or a surfactant such as Triton X-100™, etc. When the protein is secreted in the culture broth, the supernatant can be separated, after completion of the cultivation, from the bacteria or cell to collect the supernatant by a publicly known method.

The protein contained in the supernatant or the extract thus obtained can be purified by appropriately combining the publicly known methods for separation and purification. Such publicly known methods for separation and purification include a method utilizing difference in solubility such as salting out, solvent precipitation, etc.; a method mainly utilizing difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the protein thus obtained is in a free form, the protein can be converted into the salt by publicly known methods or modifications thereof. On the other hand, when the protein is obtained in the form of a salt, it can be converted into the free form or in the form of a different salt by publicly known methods or modifications thereof.

The protein produced by the recombinant can be treated, prior to or after the purification, with an appropriate protein-modifying enzyme so that the protein can be subjected to addition of an appropriate modification or removal of a partial polypeptide. Examples of the protein-modifying enzyme include trypsin, chymotrypsin, arginyl endopeptidase, protein kinase, glycosidase and the like.

The presence of the thus produced protein of the present invention can be determined by an enzyme immunoassay or western blotting using a specific antibody.

The antibody against the protein or partial peptide used in the present invention or its salts may be any of polyclonal and monoclonal antibodies so long as it recognizes the protein or partial peptide used in the present invention or its salts.

The antibody against the protein or partial peptide used in the present invention or its salts (hereinafter they are sometimes briefly referred to as the protein of the present invention) can be manufactured by publicly known methods for manufacturing antibodies or antisera.

### [Preparation of Monoclonal Antibody]

### (a) Preparation of Monoclonal Antibody-Producing Cells

The protein of the present invention is administered to warm-blooded animals either solely or together with carriers or diluents to the site where the production of antibody is possible by the administration. In order to potentiate the antibody productivity upon the administration, complete Freund's adjuvants or incomplete Freund's adjuvants may be administered. The administration is usually carried out once every about 2 to about 6 weeks and about 2 to about 10 times in total. Examples of the applicable warm-blooded animals are monkeys, rabbits, dogs, guinea pigs, mice, rats, sheep, goats and fowl, with the use of mice and rats being preferred.

In the preparation of monoclonal antibody-producing cells, a warm-blooded animal, e.g., mouse, immunized with an antigen wherein the antibody titer is noted is selected, then spleen or lymph node is collected after 2 to 5 days from the final immunization and antibody-producing cells contained therein are fused with myeloma cells from homozoic or heterozoic animal to give monoclonal antibody-producing hybridomas. Measurement of the antibody titer in antisera may be carried out, for example, by reacting a labeled protein, which will be described later, with the antiserum followed by assaying the binding activity of the labeling agent bound to the antibody. The fusion may be carried out, for example, by the known method by Koehler and Milstein [Nature, 256, 495, (1975)]. Examples of the fusion accelerator are polyethylene glycol (PEG), Sendai virus, etc., of which PEG is preferably employed.

Examples of the myeloma cells are those collected from warm-blooded animals such as NS-1, P3U1, SP2/0, AP-1, etc. In particular, P3U1 is preferably employed. A preferred ratio of the count of the antibody-producing cells used (spleen cells) to the count of myeloma cells is within a range of approximately 1:1 to 20:1. When PEG (preferably, PEG 1000 to PEG 6000) is added in a concentration of approximately 10 to 80% followed by incubation at 20 to 40° C., preferably at 30 to 37° C. for 1 to 10 minutes, an efficient cell fusion can be carried out.

Various methods can be used for screening of monoclonal antibody-producing hybridomas. Examples of such methods include a method which comprises adding the supernatant of a hybridoma to a solid phase (e.g., a microplate) adsorbed with the protein as an antigen directly or together with a carrier, adding an anti-immunoglobulin antibody (where mouse cells are used for the cell fusion, anti-mouse immunoglobulin antibody is used) or Protein A labeled with a radioactive substance or an enzyme and detecting the monoclonal antibody bound to the solid phase, and a method which comprises adding the supernatant of hybridoma to a solid phase adsorbed with an anti-immunoglobulin antibody or Protein A, adding the protein labeled with a radioactive substance or an enzyme and detecting the monoclonal antibody bound to the solid phase, or the like.

The monoclonal antibody can be screened according to publicly known methods or their modifications. In general, the screening can be performed in a medium for animal cells supplemented with HAT (hypoxanthine, aminopterin and thymidine). Any screening and growth medium can be employed as far as the hybridoma can grow there. For example, RPMI 1640 medium containing 1 to 20%, preferably 10 to 20% fetal bovine serum, GIT medium (Wako Pure Chemical Industries, Ltd.) containing 1 to 10% fetal bovine serum, a serum free medium for cultivation of a hybridoma (SFM-101, Nissui Seiyaku Co., Ltd.) and the like, can be used for the screening and growth medium. The culture is carried out generally at 20 to 40° C., preferably at 37° C., for about 5 days to about 3 weeks, preferably 1 to 2 weeks, normally in 5% CO₂. The antibody titer of the culture supernatant of a hybridoma can be determined as in the assay for the antibody titer in antisera described above.

### (b) Purification of Monoclonal Antibody

Separation and purification of a monoclonal antibody can be carried out by publicly known methods, such as separation and purification of immunoglobulins [for example, salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, Protein A or Protein G and dissociating the binding to obtain the antibody.]

### [Preparation of Polyclonal Antibody]

The polyclonal antibody of the present invention can be manufactured by publicly known methods or modifications thereof. For example, a warm-blooded animal is immunized with an immunogen (protein antigen) per se, or a complex of immunogen and a carrier protein is formed and the animal is immunized with the complex in a manner similar to the method described above for the manufacture of monoclonal antibodies. The product containing the antibody against the protein of the present invention is collected from the immunized animal followed by separation and purification of the antibody.

In the complex of immunogen and carrier protein used to immunize a warm-blooded animal, the type of carrier protein and the mixing ratio of carrier to hapten may be any type and in any ratio, as long as the antibody is efficiently produced to the hapten immunized by crosslinking to the carrier. For example, bovine serum albumin, bovine thyroglobulin or hemocyanin is coupled to hapten in a carrier-to-hapten weight ratio of approximately 0.1 to 20, preferably about 1 to 5.

A variety of condensation agents can be used for the coupling of carrier to hapten. Glutaraldehyde, carbodiimide, maleimide activated ester and activated ester reagents containing thiol group or dithiopyridyl group are used for the coupling.

The condensation product is administered to warm-blooded animals either solely or together with carriers or diluents to the site that can produce the antibody by the administration. In order to potentiate the antibody productivity upon the administration, complete Freund's adjuvant or incomplete Freund's adjuvant may be administered. The administration is usually made once every about 2 to 6 weeks and about 3 to 10 times in total.

The polyclonal antibody can be collected from the blood, ascites, etc., preferably from the blood of warm-blooded animal immunized by the method described above.

The polyclonal antibody titer in antiserum can be assayed by the same procedure as that for the determination of serum antibody titer described above. The separation and purification of the polyclonal antibody can be carried out, following the method for the separation and purification of immunoglobulins performed as in the separation and purification of monoclonal antibodies described hereinabove.

The antisense polynucleotide having an entire or part of nucleotide sequence complementary or substantially complementary to a nucleotide sequence of the polynucleotide encoding the protein or partial peptide used in the present invention (preferably DNA) (hereinafter these DNAs are sometimes collectively referred to as the DNA of the present invention in the description of antisense polynucleotide) can be any antisense polynucleotide, so long as it possesses an entire or part of nucleotide sequence complementary or substantially complementary to the nucleotide sequence of the DNA of the present invention and capable of suppressing the expression of said DNA, and an antisense DNA is preferred.

The nucleotide sequence substantially complementary to the DNA of the present invention includes, for example, a nucleotide sequence having at least about 70% homology, preferably at least about 80% homology, more preferably at least about 90% homology and most preferably at least about 95% homology, to the entire nucleotide sequence or to its partial nucleotide sequence (i.e., complementary strand to the DNA of the present invention), and the like. Especially in the entire nucleotide sequence of the complementary strand to the DNA of the present invention, preferred are (i) an antisense polynucleotide having at least about 70% homology, preferably at least about 80% homology, more preferably at least about 90% homology and most preferably at least about 95% homology, to the complementary strand of the nucleotide sequence which encodes the N-terminal region of the protein of the present invention (e.g., the nucleotide sequence around the initiation codon) when the antisense polynucleotide is directed to translation inhibition and (ii) an antisense polynucleotide having at least about 70% homology, preferably at least about 80% homology, more preferably at least about 90% homology and most preferably at least about 95% homology, to the complementary strand of the entire nucleotide sequence of the DNA of the present invention including intron, when the antisense polynucleotide is directed to RNA degradation by RNaseH, respectively.

Specific examples include an antisense polynucleotide comprising the entire or part of a nucleotide sequence complementary or substantially complementary to a nucleotide sequence of DNA comprising the nucleotide sequence represented by SEQ ID NO: 2, preferably an antisense polynucleotide comprising the entire or part of a nucleotide sequence complementary to a nucleotide sequence of DNA comprising the nucleotide sequence represented by SEQ ID NO: 2 (more preferably, an antisense polynucleotide comprising the entire or part of a nucleotide sequence complementary to a nucleotide sequence of DNA comprising the nucleotide sequence represented by SEQ ID NO: 2), etc.

The antisense polynucleotide is generally constituted by bases of about 10 to about 40, preferably about 15 to about 30.

To prevent digestion with a hydrolase such as nuclease, etc., the phosphoric acid residue (phosphate) of each nucleotide that constitutes the antisense DNA may be substituted with chemically modified phosphoric acid residues, e.g., phosphorothioate, methyl phosphonate, phosphorodithionate, etc. Also, the sugar (deoxyribose) in each nucleotide may be replaced by a chemically modified structure such as 2′-O-methylation, etc. The base part (pyrimidine, purine) may also be chemically modified and may be any one which hybridizes to a DNA containing the nucleotide sequence represented by SEQ ID NO: 2. These antisense polynucleotides may be synthesized using a publicly known DNA synthesizer, etc.

According to the present invention, the antisense polynucleotide (nucleic acid) capable of inhibiting the replication or expression of a gene for the protein of the present invention can be designed and synthesized based on the nucleotide sequence information of cloned or identified protein-encoding DNA. Such an antisense polynucleotide is hybridizable to RNA of a gene for the protein of the present invention to inhibit the synthesis or function of said RNA or is capable of modulating and/or controlling the expression of a gene for the protein of the present invention via interaction with RNA associated with the protein of the present invention. Polynucleotides complementary to the selected sequences of RNA associated with the protein of the present invention and polynucleotides specifically hybridizable to RNA associated with the protein of the present invention are useful in modulating/controlling the in vivo and in vitro expression of the protein gene of the present invention, and are useful for the treatment or diagnosis of diseases, etc. The term “corresponding” is used to mean homologous to or complementary to a particular sequence of the nucleotide including the gene, nucleotide sequence or nucleic acid. The term “corresponding” between nucleotides, nucleotide sequences or nucleic acids and proteins usually refer to amino acids of a protein (under the order) derived from the sequence of nucleotides (nucleic acids) or their complements. In the protein genes, the 5′ end hairpin loop, 5′ end 6-base-pair repeats, 5′ end untranslated region, polypeptide translation initiation codon, protein coding region, ORF translation termination codon, 3′ end untranslated region, 3′ end palindrome region, and 3′ end hairpin loop, may be selected as preferred target regions, though any other region may be selected as a target in the protein genes.

The relationship between the targeted nucleic acids and the polynucleotides complementary to at least a part of the target region, specifically the relationship between the target nucleic acids and the polynucleotides hybridizable to the target region, can be denoted to be “antisense.” Examples of the antisense polynucleotides include polynucleotides containing 2-deoxy-D-ribose, polynucleotides containing D-ribose, any other type of polynucleotides which are N-glycosides of a purine or pyrimidine base, or other polymers containing non-nucleotide backbones (e.g., commercially available protein nucleic acids and synthetic sequence-specific nucleic acid polymers) or other polymers containing nonstandard linkages (provided that the polymers contain nucleotides having such a configuration that allows base pairing or base stacking, as is found in DNA or RNA), etc. The antisense polynucleotides may be double-stranded DNA, single-stranded DNA, double-stranded RNA, single-stranded RNA or a DNA:RNA hybrid, and may further include unmodified polynucleotides (or unmodified oligonucleotides), those with publicly known types of modifications, for example, those with labels known in the art, those with caps, methylated polynucleotides, those with substitution of one or more naturally occurring nucleotides by their analogue, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (nucleases, nuclease inhibitors, toxins, antibodies, signal peptides, poly-L-lysine, etc.), saccharides (e.g., monosaccharides, etc.), those with intercalators (e.g., acridine, psoralen, etc.), those containing chelators (e.g., metals, radioactive metals, boron, oxidative metals, etc.), those containing alkylating agents, those with modified linkages (e.g., a anomeric nucleic acids, etc.), and the like. Herein the terms “nucleoside”, “nucleotide” and “nucleic acid” are used to refer to moieties that contain not only the purine and pyrimidine bases, but also other heterocyclic bases, which have been modified. Such modifications may include methylated purines and pyrimidines, acylated purines and pyrimidines and other heterocyclic rings. Modified nucleotides and modified nucleotides also include modifications on the sugar moiety, wherein, for example, one or more hydroxyl groups may optionally be substituted with a halogen atom(s), an aliphatic group(s), etc., or may be converted into the corresponding functional groups such as ethers, amines, or the like.

The antisense polynucleotide of the present invention is RNA, DNA or a modified nucleic acid (RNA, DNA). Specific examples of the modified nucleic acid include sulfur and thiophosphate derivatives of nucleic acids and those resistant to degradation of polynucleoside amides or oligonucleoside amides. The antisense polynucleotide of the present invention can be modified preferably based on the following design, that is, by increasing the intracellular stability of the antisense polynucleotide, enhancing the cell permeability of the antisense polynucleotide, increasing the affinity of the nucleic acid to the targeted sense strand to a higher level, or minimizing the toxicity, if any, of the antisense polynucleotide. An increasing number of these modifications are reported in, e.g., Pharm. Tech. Japan, 8, 247 or 395, 1992, Antisense Research and Applications, CRC Press, 1993, etc.

The antisense polynucleotide of the present invention may contain altered or modified sugars, bases or linkages. The antisense polynucleotide may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached to the nucleic acid at the 3′ or 5′ ends thereof and may also be attached thereto through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include, but are not limited to, hydroxyl protecting groups known in the art, including glycols such as polyethylene glycol, tetraethylene glycol and the like.

The inhibitory activity of the antisense polynucleotide can be examined using the transformant of the present invention, the gene expression system of the present invention in vivo and in vitro, or the translation system for the protein of the present invention in vivo and in vitro.

Hereinafter, the protein of the present invention, its partial peptides, or salts thereof (hereinafter sometimes merely referred to as the protein of the present invention), the polynucleotide (e.g., DNA) (hereinafter sometimes merely referred to as the DNA of the present invention) encoding the protein of the present invention or its partial peptides, the antibodies to the protein of the present invention, its partial peptides, or salts thereof (hereinafter sometimes referred to as the antibodies of the present invention) and the antisense polynucleotides to the polynucleotide of the present invention (e.g., DNA) (hereinafter sometimes merely referred to as the antisense polynucleotides of the present invention) are specifically described for their applications.

### (1) Screening of Drug Candidate Compound for Disease

The protein of the present invention is expressed at elevated level in cancer cells and has the acyl-CoA synthetase activity. By inhibiting the activity or expression of the protein of the present invention, the upregulated apoptosis pathway in cancer cells is activated and apoptosis of cancer cells is induced. Accordingly, the compound or its salt that inhibits the activity of the protein of the present invention can be used as a low toxic agent for preventing/treating cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, and so on.

Accordingly, the protein of the present invention is useful as a reagent for screening the compound or its salts that inhibit the activity of the protein of the present invention.

In other words, the present invention provides a method of screening the compound or its salts that inhibit the activity of the protein of the present invention (e.g., acyl-CoA synthetase activity, etc.), which comprises using the protein of the present invention.

Specific examples include the following methods, etc.

The compound or its salts that inhibit the activity of the protein of the present invention is screened by comparing (i) the acyl-CoA synthetase activity of the protein of the present invention with (ii) the acyl-CoA synthetase activity of a mixture of the protein of the present invention and a test compound.

(1) The compound or its salts that inhibit the activity of the protein of the present invention is screened by assaying the acyl-CoA synthetase activities (i) when the protein of the present invention is reacted with a fatty acid labeled with a radioisotope or a fluorescent substance, and (ii) when the protein of the present invention is reacted with a fatty acid labeled with a radioisotope or a fluorescent substance in the presence of a test compound, respectively.

The reaction is carried out in an appropriate buffer solution. After the enzyme reaction, the reaction product is separated by, e.g., partition between aqueous and organic solvent layers, etc. and extracting acyl-CoA dissolved in the aqueous layer. The radioactivity or fluorescence intensity of acyl-CoA is assayed by publicly known methods using a scintillation counter, fluorography, etc. Examples of the radioisotope are [¹²⁵I], [¹³¹I], [³H], [¹⁴C], [³²P], [³³P], [³⁵S], etc., and examples of the fluorescent substance include cyanine fluorescent substances (e.g., Cy2, Cy3, Cy5, Cy5.5, Cy7 (Amersham Biosciences)), fluorescamine, fluorescein isothiocyanate, NBD (7-nitrobenz-2-oxa-1,3-diazol), BODIPY (boron-dipyrromethene), etc.

(2) The compound or its salts that inhibit the activity of the protein of the present invention is screened by assaying the acyl-CoA synthetase activities (i) when the protein of the present invention is reacted with a fatty acid labeled with a radioisotope, and (ii) when the protein of the present invention is reacted with a fatty acid labeled with a radioisotope in the presence of a test compound, respectively.

Specifically, the protein of the present invention is reacted in 0.5 ml of a solution containing 0.2 mM Tris-HCl buffer (pH7.5), 2.5 mM ATP, 8 mM MgCl₂, 2 mM EDTA, 20 mM NaF, 0.1% (w/v) Triton X-100, 10 μM [1-¹⁴C] palmitate (5 μCi/μl) and 0.5 mM coenzyme A at 35° C. for 10 minutes, in the absence or presence of a test compound. The reaction is initiated by adding CoA and stopped by adding 2.5 ml of isopropanol:n-heptane:1M sulfuric acid (40:10:1, v/v). After the reaction is stopped, 0.5 ml of water and 2.5 ml of n-heptane are added to remove the organic solvent layer containing the unreacted fatty acids. The aqueous layer is further washed 3 times with 2.5 ml of n-heptane and the radioactivity retained in the aqueous layer is counted according to publicly known methods using a scintillation counter, etc.

(3) The compound or its salts that inhibit the activity of the protein of the present invention is screened by assaying the acyl-CoA synthetase activities (i) when the protein of the present invention is reacted with a fatty acid labeled with a fluorescent substance, and (ii) when the protein of the present invention is reacted with a fatty acid labeled with a fluorescent substance in the presence of a test compound, respectively.

Specifically, the protein of the present invention is reacted in 0.5 ml of a solution containing 0.2 mM Tris-HCl buffer (pH7.5), 2.5 mM ATP, 8 mM MgCl₂, 2 mM EDTA, 20 mM NaF, 0.1% (w/v) Triton X-100, 10 μM C₁-BODIPY-C₁₂ (4,4-difluoro-5-methyl-4-bora-3a,4a-diaza-s-indacene-3-dodecanoic acid) and 0.5 mM coenzyme A at 35° C. for 10 minutes, in the absence or presence of a test compound. The reaction is initiated by adding CoA and stopped by adding 2.5 ml of isopropanol:n-heptane:1M sulfuric acid (40:10:1, v/v). After the reaction is stopped, 0.5 ml of water and 2.5 ml of n-heptane are added to remove the organic solvent layer containing the unreacted fatty acids. The aqueous layer is further washed 3 times with 2.5 ml of n-heptane and the fluorescence intensity retained in the aqueous layer is counted according to publicly known methods using fluorography, etc.

Preferably, the protein of the present invention described above is the protein produced by culturing transformants bearing the DNA encoding the protein of the present invention. Furthermore, cells capable of expressing the protein of the present invention may be used in the reaction in a similar manner.

As the cells capable of producing the protein of the present invention, there are used, for example, a host (transformant) transformed with a vector bearing the DNA encoding the protein of the present invention. Animal cells such as COS7 cells, CHO cells, HEK293 cells, etc., yeasts, etc. are preferably used as the host. For the screening, the transformant, in which the protein of the present invention is expressed, e.g., by culturing through the procedures described above, is preferably employed. The procedures for culturing the cells capable of expressing the protein of the present invention are similar to the culturing procedures for the transformant of the present invention described above.

Examples of the test compound include peptides, proteins, antibodies, non-peptide compounds, synthetic compounds, fermentation products, cell extracts, plant extracts, animal tissue extracts, blood plasma, and the like. These compounds may be novel or publicly known compounds. The test compounds may form salts and as salts of the test compounds, there are, for example, metal salts, ammonium salts, salts with organic bases, salts with inorganic acids, salts with organic acids, salts with basic or acidic amino acids, etc. Preferred examples of the metal salts include alkali metal salts such as sodium salts, potassium salts, etc.; alkaline earth meal salts such as calcium salts, magnesium salts, barium salts, etc.; aluminum salts, etc. Preferred examples of the salts with organic bases include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine, etc. Preferred examples of the salts with inorganic acids include salts with hydrochloric acid, hydrobromic acid, nitric acid, sulfuric acid, phosphoric acid, etc. Preferred examples of the salts with organic acids include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid, etc. Preferred examples of the salts with basic amino acids include salts with arginine, lysine, ornithine, etc., and preferred examples of the salts with acidic amino acids include salts with aspartic acid, glutamic acid, etc.

Among these salts, preferred are pharmaceutically acceptable salts. Where the compounds have, for example, acidic functional groups in the compound, the salts include inorganic salts such as alkali metal salts (e.g., sodium salts, potassium salts, etc.), alkaline earth metal salts (e.g., calcium salts, magnesium salts, barium salts, etc.), etc.; ammonium salts, etc.; and where the compounds have basic functional groups in the compound, the salts include salts with inorganic acids such as hydrobromic acid, nitric acid, sulfuric acid, phosphoric acid, etc., or salts with organic acids such as acetic acid, phthalic acid, fumaric acid, oxalic acid, tartaric acid, maleic acid, citric acid, succinic acid, methanesulfonic acid, p-toluenesulfonic acid, etc.

For example, when a test compound decreases the acyl-CoA synthetase activity in the case (ii) described above by at least about 20%, preferably at least 30% and more preferably at least about 50%, as compared to the case (i) above, the test compound can be selected as the compound that inhibits the activity of the protein of the present invention.

The compound which has the activity of inhibiting the activity of the protein of the present invention is useful as a safe and low toxic medicament including an agent for preventing/treating, e.g., cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, and so on.

The compound or its salt, which is obtained by using the screening method or screening kit of the present invention, includes a compound selected from, for example, peptides, proteins, antibodies, non-peptide compounds, synthetic compounds, fermentation products, cell extracts, plant extracts, animal tissue extracts, blood plasma, etc. These compounds may be novel or publicly known compounds. Salts of these compounds used are those given above as the salts of test compounds.

In addition, the gene encoding the protein of the present invention is expressed at elevated level in cancer cells and produces the protein of the present invention having the acyl-CoA synthetase activity. By inhibiting the expression of this gene, the upregulated apoptosis pathway in cancer cells is activated to induce apoptosis of cancer cells. Accordingly, the compound or its salts that inhibit the expression of this gene can also be used as an agent for preventing/treating, e.g., cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, and so on.

Therefore, the DNA of the present invention is useful as a reagent for screening the compound or its salts that inhibit the expression of the gene encoding the protein of the present invention.

The screening method includes a method of screening which comprises comparing (iii) the case where a cell capable of producing the protein of the present invention is cultured and (iv) the case where a cell capable of producing the protein used in the present invention is cultured in the presence of a test compound.

In the screening method described above, the expression level of the gene described above (specifically, the level of the protein of the present invention or the level of mRNA encoding the said protein) is determined in the cases of (iii) and (iv), followed by comparison.

Examples of the test compound and the cells capable of producing the protein of the present invention are the same as described above.

The protein level can be determined by publicly known methods, e.g., by measuring the aforesaid protein present in the cell extract, etc., using an antibody capable of recognizing the protein of the present invention, in accordance with methods such as western blot analysis, ELISA, etc., or their modifications.

The mRNA level can be determined by publicly known methods, e.g., in accordance with methods such as northern hybridization using a nucleic acid containing the entire or a part of SEQ ID NO: 2 as a probe, or PCR using a nucleic acid containing the entire or a part of SEQ ID NO: 2 as a primer, or their modifications.

For example, when a test compound inhibits the expression of the gene in the case (iv) described above by at least about 20%, preferably at least 30% and more preferably at least about 50%, as compared to the case (iii) above, the test compound can be selected to be a compound capable of suppressing (inhibiting) the expression of the gene encoding the protein of the present invention.

The screening kit of the present invention comprises the protein used in the present invention, its partial peptide or salts thereof, or the cell capable of producing the protein used in the present invention, or its partial peptide.

The compound or its salts obtained by using the screening method or screening kit of the present invention is the test compound described above, e.g., a compound selected from peptides, proteins, antibodies, non-peptide compounds, synthetic compounds, fermentation products, cell extracts, plant extracts, animal tissue extracts, plasma, etc., or its salt, and is a compound or its salts that inhibit the activity of the protein of the present invention (e.g., acyl-CoA synthetase activity, etc.), or inhibit the expression of the gene for said protein or inhibit the expression of the protein of the present invention.

Salts of these compounds used are the same as the salts of the test compounds described above.

The compound or its salt that inhibits the activity of the protein of the present invention, the compound or its salt that inhibits the gene expression of the protein of the present invention, and the compound or its salt that inhibits the expression of the protein of the present invention are useful as medicaments including agents for preventing/treating, for example, cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), apoptosis promoters in cancer cells, cancer cell growth inhibitors, agents for suppressing the metastasis/relapse of cancer, and the like.

Where the compound or its salt obtained by using the screening method or screening kit of the present invention is used as the preventive/therapeutic agent described above, the compound can be prepared into pharmaceutical preparations in a conventional manner.

For example, the composition for oral administration includes solid or liquid preparations, specifically, tablets (including dragees and film-coated tablets), pills, granules, powdery preparations, capsules (including soft capsules), syrup, emulsions, suspensions, etc. Such a composition is manufactured by publicly known methods and contains a vehicle, a diluent or excipient conventionally used in the field of pharmaceutical preparations. Examples of the vehicle or excipient for tablets are lactose, starch, sucrose, magnesium stearate, etc.

Examples of the composition for parenteral administration are injectable preparations, suppositories, etc. The injectable preparations may include dosage forms such as intravenous, subcutaneous, intracutaneous and intramuscular injections, drip infusions, intraarticular injections, etc. These injectable preparations may be prepared by methods publicly known. For example, the injectable preparations may be prepared by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mols) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is usually filled in an appropriate ampoule. The suppository used for rectal administration may be prepared by blending the aforesaid antibody or its salt with conventional bases for suppositories.

Advantageously, the oral or parenteral pharmaceutical compositions described above are prepared into pharmaceutical preparations with a unit dose suitable for a dose of the active ingredient. Such unit dose preparations include, for example, tablets, pills, capsules, injections (ampoules), suppositories, etc. The amount of the aforesaid compound contained is generally 5 to 500 mg per dosage unit form; it is preferred that the aforesaid antibody is contained in about 5 to about 100 mg especially in the form of injection, and in 10 to 250 mg for the other forms.

Each of the compositions described above may further contain other active components unless formulation causes any adverse interaction with the compound described above.

Since the pharmaceutical preparations thus obtained are safe and low toxic, they can be administered to human or warm-blooded animal (e.g., mouse, rat, rabbit, sheep, swine, bovine, horse, fowl, cat, dog, monkey, chimpanzee, etc.) orally or parenterally.

The dose of the above compound or its salt may vary depending upon its effect, target disease, subject to be administered, route of administration, etc. For example, when the compound or its salt that inhibits the activity of the protein of the present invention is orally administered for the purpose of treating, for example, lung cancer, the compound or its salt is generally administered to an adult (as 60 kg body weight) in a daily dose of about 0.1 to about 100 mg, preferably about 1.0 to about 50 mg and more preferably about 1.0 to about 20 mg. In parenteral administration, a dose of the said compound or its salt may vary depending upon subject to be administered, target disease, etc. When the compound or its salt that inhibits the activity of the protein of the present invention is administered to an adult (as 60 kg body weight) in the form of an injectable preparation for the purpose of treating, e.g., lung cancer, it is advantageous to administer the compound or its salt by way of injection in a daily dose of about 0.01 to about 30 mg, preferably about 0.1 to about 20 mg, and more preferably about 0.1 to about 10 mg. For other animal species, the corresponding dose as converted per 60 kg weight can be administered.

The compound described above can be used in combination with a hormonal therapeutic drug, an anticancer agent (e.g., a chemotherapeutic agent, an immunotherapeutic agent or an agent inhibiting the action of cell growth factors or their receptors) (hereinafter referred to as a concomitant drug). When the concomitant drug is used, a dosing period is not restricted; the compound of the present invention or its pharmaceutical composition and the concomitant drug or its pharmaceutical composition may be administered to the subject to be administered either simultaneously or at certain time intervals. The dose of the concomitant drug may be modified according to the dose used clinically and may be appropriately chosen depending upon subject to be administered, route for administration, condition, combination, etc.

Examples of the “hormonal therapeutic agent” include fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, Tadenan, mepartricin, raloxifene, ormeloxifene, levormeloxifene, anti-estrogens (e.g., tamoxifen citrate, toremifene citrate, etc.), ER down-regulator (e.g., fulvestrant, etc.), human menopausal gonadotropin, follicle stimulating hormone, pill preparations, mepitiostane, testrolactone, aminoglutethimide, LH-RH agonists (e.g., goserelin acetate, buserelin, Leuprorelin, etc.), droloxifene, epitiostanol, ethinylestradiol sulfonate, aromatase inhibitors (e.g., fadrozole hydrochloride, anastrozole, retrozole, exemestane, vorozole, formestane, etc.), anti-androgens (e.g., flutamide, bicartamide, nilutamide, etc.), 5α-reductase inhibitors (e.g., finasteride, dutasteride, epristeride, etc.), adrenocorticohormone drugs (e.g., dexamethasone, prednisolone, betamethasone, triamcinolone, etc.), androgen synthesis inhibitors (e.g., abiraterone, etc.), retinoid and drugs that retard retinoid metabolism (e.g., liarozole, etc.), and among them, LH-RH agonists (e.g., goserelin acetate, buserelin, Leuprorelin, etc.) are preferable.

Examples of the “chemotherapeutic agent” include alkylating agents, antimetabolites, anticancer antibiotics, plant-derived antitumor agents, etc.

Examples of the “alkylating agents” include nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine sodium phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin, etc.

Examples of the “antimetabolites” include mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur, etc.), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine, etc.

Examples of the “anticancer antibiotics” include actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, etc.

Examples of the “plant-derived anticancer agents” include etoposide, etoposide phosphate, vinblastine sulfate, vincristine sulfate, vindesine sulfate, teniposide, paclitaxel, docetaxel, vinorelbine, etc.

Examples of the “immunotherapeutic agents (BRM)” include picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, etc.

The “growth factor” in the “agent inhibiting the action of cell growth factors or their receptors” may be any substance so long as it promotes the growth of cells, which is normally a peptide having a molecular weight of not greater than 20,000 and capable of exhibiting its activity at a low concentration through binding to the receptor; specifically, the growth factor includes (1) EGF (epidermal growth factor) or a substance having substantially the same activity as EGF [e.g., EGF, heregulin (HER2 ligand), etc.], (2) insulin or a substance having substantially the same activity as insulin [e.g., insulin, IGF (insulin-like growth factor)-1, IGF-2, etc.], (3) FGF (fibroblast growth factor) or a substance having substantially the same activity as FGF [e.g., acidic FGF, basic FGF, KGF (keratinocyte growth factor), FGF-10, etc.], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), etc.], and the like.

Examples of the “receptor of cell growth factor” may be any substabce so long as it is a receptor capable of binding to the aforesaid cell growth factor, specifically including EGF receptor, heregulin receptor (HER2), insulin receptor, IGF receptor, FGF receptor-1 or FGF receptor-2, and the like.

Examples of the “agent inhibiting the action of cell growth factors” include trastuzumab (Herceptin (trade mark); HER2 antibody), imatinib mesilate, ZD1839 or cetuximab, antibody against VEGF (e.g., bevacizumab), antibody against VEGF receptor, gefitinib, erlotinib, and the like.

In addition to the drugs described above, L-asparaginase, aceglatone, procarbazine hydrochloride, protoporphyrin-cobalt complex, mercuric hematoporphyrin-sodium, topoisomerase I inhibitors (e.g., irinotecan, topotecan, etc.), topoisomerase II inhibitors (e.g., sobuzoxane, etc.), differentiation inducers (e.g., retinoid, vitamin D, etc.), angiogenesis inhibitors (e.g., thalidomide, SU11248, etc.), α-blockers (e.g., tamsulosin hydrochloride, naftopidil, urapidil, alfuzosin, terazosin, prazosin, silodosin, etc.), serine/threonine kinase inhibitors, endothelin receptor antagonists (e.g., atrasentan, etc.), proteasome inhibitors (e.g., bortezomib, etc.), Hsp 90 inhibitors (e.g., 17-AAQ etc.), spironolactone, minoxidil, 11α-hydroxyprogesterone, bone resorption inhibiting/metastasis suppressing agents (e.g., zoledronic acid, alendronic acid, pamidronic acid, etidronic acid, ibandronic acid, clodronic acid) and the like can be used.

(2) Quantification of the Protein of the Invention, its Partial Peptide, or their Salts

The antibody against the protein of the present invention (hereinafter sometimes merely referred to as the antibody of the present invention) is capable of specifically recognizing the protein of the present invention and can thus be used for quantification of the protein of the present invention in a test fluid, in particular, for quantification by sandwich immunoassay; etc.

That is, the present invention provides:

(i) a method of quantifying the protein of the present invention in a test fluid, which comprises competitively reacting the antibody of the present invention, a test fluid and a labeled form of the protein of the present invention, and measuring the proportion of the labeled protein of the present invention bound to said antibody; and,
(ii) a method of quantifying the protein of the present invention in a test fluid, which comprises reacting a test fluid simultaneously or continuously with the antibody of the present invention immobilized on a carrier and another labeled antibody of the present invention, and then measuring the activity of the labeling agent on the insoluble carrier.

In the quantification method (ii) described above, it is preferred that one antibody is capable of recognizing the N-terminal region of the protein of the present invention, while another antibody is capable of reacting with the C-terminal region of the protein of the present invention.

The monoclonal antibody against the protein of the present invention (hereinafter sometimes referred to as the monoclonal antibody of the present invention) can be used to quantify the protein of the present invention. In addition, the protein can be detected by means of a tissue staining as well. For these purposes, the antibody molecule per se may be used or F (ab′)₂, Fab′ or Fab fractions of the antibody molecule may also be used.

The method of quantifying the protein of the present invention using the antibody of the present invention is not to be particularly limited. Any quantification method can be used, so long as the amount of antibody, antigen or antibody-antigen complex corresponding to the amount of antigen (e.g., the amount of the protein) in a test fluid can be detected by chemical or physical means and the amount of the antigen can be calculated from a standard curve prepared from standard solutions containing known amounts of the antigen. For such an assay method, for example, nephrometry, the competitive method, the immunometric method, the sandwich method, etc. are suitably used and in terms of sensitivity and specificity, it is particularly preferred to use the sandwich method described later.

Examples of the labeling agent available for the assay method using the labeling substance are radioisotopes (e.g., [¹²⁵I], [¹³¹I], [³H], [¹⁴C], [³²P], [³³P], [³⁵S], etc.), fluorescent substances [e.g., cyanine fluorescent substances (e.g., Cy2, Cy3, Cy5, Cy5.5, Cy7 (Amersham Biosciences)), fluorescamine, fluorescein isothiocyanate, NBD (7-nitrobenz-2-oxa-1,3-diazol), BODIPY (boron-dipyrromethene)], enzymes (e.g., β-galactosidase, β-glucosidase, alkaline phosphatase, peroxidase, malate dehydrogenase), luminescent substances (e.g., luminol, luminal derivatives, luciferin, lucigenin), biotin, lanthanide elements, etc. The biotin-avidin system may be used as well for binding of an antibody or antigen to a labeling agent.

For immobilization of the antigen or antibody, physical adsorption may be used. Chemical binding techniques conventionally used for insolubilization or immobilization of proteins, enzymes, etc. may also be used. For carriers, there are used, e.g., insoluble polysaccharides such as agarose, dextran, cellulose, etc.; synthetic resin such as polystyrene, polyacrylamide, silicon, etc., glass and the like.

In the sandwich method, the immobilized monoclonal antibody of the present invention is reacted with a test fluid (primary reaction), then with a labeled form of another monoclonal antibody of the present invention (secondary reaction), and the activity of the label on the immobilizing carrier is measured, whereby the amount of the protein of the present invention in the test fluid can be quantified. The order of the primary and secondary reactions may be reversed, and the reactions may be performed simultaneously or with an interval. The methods of labeling and immobilization can be performed by the methods described above. In the immunoassay by the sandwich method, the antibody used for immobilized or labeled antibodies is not necessarily one species, but a mixture of two or more species of antibody may be used to increase the measurement sensitivity.

In the methods of assaying the protein of the present invention by the sandwich method of the present invention, antibodies that bind to different sites of the protein of the present invention are preferably used as the monoclonal antibodies of the present invention used for the primary and secondary reactions. That is, in the antibodies used for the primary and secondary reactions are, for example, when the antibody used in the secondary reaction recognizes the C-terminal region of the protein of the present invention, it is preferable to use the antibody recognizing the region other than the C-terminal region for the primary reaction, e.g., the antibody recognizing the N-terminal region.

The monoclonal antibodies of the present invention can be used for the assay systems other than the sandwich method, for example, the competitive method, the immunometric method, nephrometry, etc.

In the competitive method, antigen in a test fluid and the labeled antigen are competitively reacted with antibody, and the unreacted labeled antigen (F) and the labeled antigen bound to the antibody (B) are separated (B/F separation). The amount of the label in B or F is measured, and the amount of the antigen in the test fluid is quantified. This reaction method includes a liquid phase method using a soluble antibody as an antibody, polyethylene glycol or a secondary antibody against the soluble antibody for B/F separation, and an immobilized method either using an immobilized antibody as the primary antibody, or using a soluble antibody as the primary antibody and immobilized antibody as the secondary antibody.

In the immunometric method, antigen in a test fluid and immobilized antigen are competitively reacted with a definite amount of labeled antibody, the immobilized phase is separated from the liquid phase, or antigen in a test fluid and an excess amount of labeled antibody are reacted, immobilized antigen is then added to bind the unreacted labeled antibody against the immobilized phase, and the immobilized phase is separated from the liquid phase. Then, the amount of the label in either phase is measured to quantify the antigen in the test fluid.

In the nephrometry, insoluble precipitate produced after the antigen-antibody reaction in gel or solution is quantified. When the amount of antigen in the test fluid is small and only a small amount of precipitates is obtained, laser nephrometry using scattering of laser is advantageously employed.

For applying each of these immunological methods to the quantification method of the present invention, any particular conditions or procedures are not required. The assay system for the protein of the present invention or its salt is constructed by adding the usual technical consideration in the art to the conventional conditions and procedures. For the details of these general technical means, reference can be made to the following reviews and texts.

For example, Hiroshi Irie, ed. “Radioimmunoassay” (Kodansha, published in 1974), Hiroshi Irie, ed. “Sequel to the Radioimmunoassay” (Kodansha, published in 1979), Eiji Ishikawa, et al. ed. “Enzyme immunoassay” (Igakushoin, published in 1978), Eiji Ishikawa, et al. ed. “Immunoenzyme assay” (2nd ed.) (Igakushoin, published in 1982), Eiji Ishikawa, et al. ed. “Immunoenzyme assay” (3rd ed.) (Igakushoin, published in 1987), Methods in ENZYMOLOGY, Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays)), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies))(all published by Academic Press Publishing), etc.

As described above, the protein of the present invention can be quantified with high sensitivity, using the antibody of the present invention.

Furthermore, when an increased or decreased level of the protein of the present invention is detected by quantifying the level of the protein of the present invention using the antibody of the present invention, it can be diagnosed that one suffers from, for example, cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), or the like; or it is highly likely to suffer from these diseases in the future.

Moreover, the antibody of the present invention can be used to detect the protein of the present invention, which is present in a test sample such as body fluids, tissues, etc. The antibody can also be used to prepare an antibody column for purification of the protein of the present invention, detect the protein of the present invention in each fraction upon purification, analyze the behavior of the protein of the present invention in the cells under investigation; etc.

### (3) Gene Diagnostic Agent

By using the DNA of the present invention, e.g., as a probe, an abnormality (gene abnormality) of the DNA or mRNA encoding the protein of the present invention or its partial peptide in human or warm-blooded animal (e.g., rat, mouse, guinea pig, rabbit, fowl, sheep, swine, bovine, horse, cat, dog, monkey, chimpanzee, etc.) can be detected. Therefore, the DNA of the present invention is useful as a gene diagnostic agent for detecting damages to the DNA or mRNA, its mutation, or decreased expression, increased expression or overexpression, etc. of the DNA or mRNA, and so on.

The gene diagnosis described above using the DNA of the present invention can be performed by, for example, the publicly known northern hybridization assay or the PCR-SSCP assay (Genomics, 5, 874-879 (1989); Proceedings of the National Academy of Sciences of the United States of America, 86, 2766-2770 (1989)), etc.

Where an overexpression or decreased expression is detected by, e.g., northern hybridization or DNA mutation is detected by the PCR-SSCP assay, it can be diagnosed that it is highly likely to suffer from, for example, cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), or the like.

### (4) Medicament Comprising Antisense Polynucleotide

The antisense polynucleotide of the present invention capable of complementarily binding to the DNA of the present invention and inhibiting expression of the DNA has an apoptosis promoting activity, growth inhibitory action, etc. in cancer cells and is low toxic. Moreover, the antisense polynucleotide can suppress the functions of the protein of the present invention or the DNA of the present invention in vivo (e.g., acyl-CoA synthetase activity, etc.). Thus, the antisense polynucleotide can be used as an agent for preventing/treating, for example, cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, or the like.

Where the antisense polynucleotide described above is used as a medicament, it can be prepared into pharmaceutical preparations by publicly known methods, and the preparations can be provided for administration.

For example, when the antisense polynucleotide described above can be administered alone, or the antisense polynucleotide is inserted into an appropriate vector such as retrovirus vector, adenovirus vector, adenovirus-associated virus vector, etc., and may then be administered orally or parenterally to human or a mammal (e.g., rat, rabbit, sheep, swine, bovine, cat, dog, monkey, etc.) in a conventional manner. The antisense polynucleotide may also be administered as it stands, or may be prepared in pharmaceutical preparations together with a physiologically acceptable carrier to assist its uptake, which are then administered by gene gun or through a catheter such as a catheter with a hydrogel. Alternatively, the antisense polynucleotide may be prepared into an aerosol, which is topically administered into the trachea as an inhaler.

Further for the purposes of improving pharmacokinetics, prolonging a half-life and improving intracellular uptake efficiency, the antisense polynucleotide described above is prepared into pharmaceutical preparations (injectable preparations) alone or together with a carrier such as liposome, etc. and the preparations may be administered intravenously, subcutaneously, intraarticularly, into the cancerous lesions, etc.

A dose of the antisense polynucleotide may vary depending on target disease, subject to be administered, route for administration, etc. For example, where the antisense polynucleotide of the present invention is administered for the purpose of treatment, the antisense polynucleotide is generally administered to an adult (60 kg body weight) in a daily dose of about 0.1 to 100 mg.

In addition, the antisense polynucleotide may also be used as an oligonucleotide probe for diagnosis to examine the presence of the DNA of the present invention in tissues or cells and states of its expression.

As in the antisense polynucleotide described above, the double-stranded RNA (siRNA (small (short) interfering RNA), shRNA (small (short) hairpin RNA), etc. to the polynucleotide of the present invention) comprising a part of RNA encoding the protein of the present invention, ribozyme comprising a part of RNA encoding the protein of the present invention, etc. can also suppress the expression of a gene for the present invention to suppress the in vivo function of the protein used in the present invention or the DNA used in the present invention, and can be used as a medicament such as an agent for preventing/treating, for example, cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, or the like.

The double-stranded RNA can be designed based on a sequence of the polynucleotide of the present invention and manufactured by modifications of publicly known methods (e.g., Nature, 411, 494, 2001).

The ribozyme can be designed based on a sequence of the polynucleotide of the present invention and manufactured by modifications of publicly known methods (e.g., TRENDS in Molecular Medicine, 7, 221, 2001). For example, the ribozyme can be manufactured by ligating a publicly known ribozyme to a part of the RNA encoding the protein of the present invention. A part of the RNA encoding the protein of the present invention includes a site (RNA fragment) in the vicinity of the cleavage site on the RNA of the present invention, which may be cleaved by a publicly known ribozyme.

Where the double-stranded RNA or ribozyme is used as the agent described above, they can be prepared into pharmaceutical preparations as in the antisense polynucleotide, and the preparations can be provided for administration.

### (5) Medicament Comprising the Antibody of the Present Invention

The antibody against the protein of the present invention, preferably, the antibody having the action of neutralizing the activity of the protein of the present invention can be used as a medicament such as an agent for preventing/treating, for example, cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, or the like.

Since the aforesaid preventive/therapeutic agent for diseases comprising the antibody of the present invention are low toxic, they can be administered to human or a mammal (e.g., rat, rabbit, sheep, swine, bovine, cat, dog, monkey, etc.) orally or parenterally (e.g., intravascularly) either as liquid preparations as they are or as pharmaceutical compositions of adequate dosage form.

The dose for administration of the antibody of the present invention may vary depending upon subject to be administered, target disease, conditions, route of administration, etc. For example, when the antibody of the present invention is orally administered for the purpose of treating, for example, lung cancer, the antibody is generally administered to an adult (as 60 kg body weight) in a daily dose of about 0.1 to about 1000 mg, preferably about 1.0 to about 300 mg and more preferably about 3.0 to about 50 mg. In parenteral administration, a dose of the said antibody may vary depending upon subject to be administered, target disease, conditions, route of administration, etc. When the antibody of the present invention is administered to an adult (as 60 kg body weight) in the form of an injectable preparation for the purpose of treating, for example, lung cancer, it is advantageous to administer the antibody by way of injection in a daily dose of about 0.01 to about 30 mg, preferably about 0.1 to about 20 mg, and more preferably about 0.1 to about 10 mg. For other animal species, the corresponding dose as converted per 60 kg weight can be administered.

The antibody of the present invention may be administered in itself or in the form of an appropriate pharmaceutical composition. The pharmaceutical composition used for the administration may contain the aforesaid antibody or its salt, pharmacologically acceptable carriers diluents or excipients. Such a composition is provided in the form of pharmaceutical preparations suitable for oral or parenteral administration (e.g., intravascular injection). The composition is preferably administered as an inhibitor.

Each composition described above may further contain other active ingredients unless the formulation causes any adverse interaction with the antibody described above.

### (6) DNA Transgenic Animal

The present invention provides a non-human mammal bearing a DNA encoding the protein of the present invention, which is exogenous (hereinafter simply referred to as the exogenous DNA of the present invention) or its variant DNA (sometimes simply referred to as the exogenous variant DNA of the present invention).

That is, the present invention provides:

(1) A non-human mammal bearing the exogenous DNA of the present invention or its variant DNA;
(2) The mammal according to (1), wherein the non-human mammal is a rodent;
(3) The mammal according to (2), wherein the rodent is mouse or rat; and,
(4) A recombinant vector containing the exogenous DNA of the present invention or its variant DNA and capable of expressing in a mammal; etc.

The non-human mammal bearing the exogenous DNA of the present invention or its variant DNA (hereinafter simply referred to as the DNA transgenic animal of the present invention) can be prepared by transfecting a desired DNA into an unfertilized egg, a fertilized egg, a spermatozoon, a germinal cell containing a primordial germinal cell thereof, or the like, preferably in the embryogenic stage in the development of a non-human mammal (more preferably in the single cell or fertilized cell stage and generally before the 8-cell phase), by standard means, such as the calcium phosphate method, the electric pulse method, the lipofection method, the agglutination method, the microinjection method, the particle gun method, the DEAE-dextran method, etc. Also, it is possible to transfect the exogenous DNA of the present invention into a somatic cell, a living organ, a tissue cell, or the like by the DNA transfection methods, and utilize the transformant for cell culture, tissue culture, etc. In addition, these cells may be fused with the above-described germinal cell by a publicly known cell fusion method to prepare the DNA transgenic animal of the present invention.

Examples of the non-human mammal that can be used include bovine, swine, sheep, goat, rabbits, dogs, cats, guinea pigs, hamsters, mice, rats, etc. Above all, preferred are rodents, especially mice (e.g., C57B1/6 strain, DBA2 strain, etc. for a pure line and for a cross line, B6C3F₁ strain, BDF₁ strain B6D2F₁ strain, BALB/c strain, ICR strain, etc.), rats (Wistar, SD, etc.) or the like, since they are relatively short in ontogeny and life cycle and are easy in breeding from a standpoint of creating model animals for human disease.

“Mammals” in a recombinant vector that can be expressed in the mammals include the aforesaid non-human mammals, human, etc.

The exogenous DNA of the present invention refers to the DNA of the present invention that is once isolated and extracted from mammals, not the DNA of the present invention inherently possessed by the non-human mammals.

The mutant DNA of the present invention includes mutants resulting from variation (e.g., mutation, etc.) in the nucleotide sequence of the original DNA of the present invention, specifically DNAs resulting from base addition, deletion, substitution with other bases, etc. and further including abnormal DNA.

The abnormal DNA is intended to mean DNA that expresses the abnormal protein of the present invention and exemplified by the DNA that expresses a protein for suppressing the function of the normal protein of the present invention.

The exogenous DNA of the present invention may be any one of those derived from a mammal of the same species as, or a different species from, the mammal as the target animal. In transfecting the DNA of the present invention into the target animal, it is generally advantageous to use the DNA as a DNA construct in which the DNA is ligated downstream a promoter capable of expressing the DNA in the target animal. For example, in the case of transfecting the human DNA of the present invention, a DNA transgenic mammal that highly expresses the DNA of the present invention, can be prepared by microinjecting a DNA construct (e.g., vector, etc.) ligated with the human DNA of the present invention into a fertilized egg of the target non-human mammal downstream various promoters which are capable of expressing the DNA derived from various mammals (e.g., rabbits, dogs, cats, guinea pigs, hamsters, rats, mice, etc.) bearing the DNA of the present invention highly homologous to the human DNA.

As expression vectors for the protein of the present invention, there are Escherichia coli-derived plasmids, Bacillus subtilis-derived plasmids, yeast-derived plasmids, bacteriophages such as λ phage, retroviruses such as Moloney leukemia virus, etc., and animal viruses such as vaccinia virus, baculovirus, etc. Of these vectors, Escherichia coli-derived plasmids, Bacillus subtilis-derived plasmids, or yeast-derived plasmids, etc. are preferably used.

Examples of these promoters for regulating the DNA expression described above include (i) promoters for DNA derived from viruses (e.g., simian virus, cytomegalovirus, Moloney leukemia virus, JC virus, breast cancer virus, poliovirus, etc.), and (ii) promoters derived from various mammals (human, rabbits, dogs, cats, guinea pigs, hamsters, rats, mice, etc.), for example, promoters of albumin, insulin II, uroplakin II, elastase, erythropoietin, endothelin, muscular creatine kinase, glial fibrillary acidic protein, glutathione S-transferase, platelet-derived growth factor β, keratins K1, K10 and K14, collagen types I and II, cyclic AMP-dependent protein kinase βI subunit, dystrophin, tartarate-resistant alkaline phosphatase, atrial natriuretic factor, endothelial receptor tyrosine kinase (generally abbreviated as Tie2), sodium-potassium adenosine triphosphorylase (Na,K-ATPase), neurofilament light chain, metallothioneins I and IIA, metalloproteinase I tissue inhibitor, MHC class I antigen (H-2L), H-ras, renin, dopamine β-hydroxylase, thyroid peroxidase (TPO), peptide chain elongation factor 1α(EF-1α), β actin, α and β myosin heavy chains, myosin light chains 1 and 2, myelin base protein, thyroglobulins, Thy-1, immunoglobulins, H-chain variable region (VNP), serum amyloid component P, myoglobin, troponin C, smooth muscle α actin, preproencephalin A, vasopressin, etc. Among them, cytomegalovirus promoters, human peptide chain elongation factor 1α (EF-1α) promoters, human and chicken β actin promoters, etc., which are capable of high expression in the whole body are preferred.

Preferably, the vectors described above have a sequence that terminates the transcription of the desired messenger RNA in the DNA transgenic animal (generally termed a terminator); for example, a sequence of each DNA derived from viruses and various mammals, and SV40 terminator of the simian virus and the like are preferably used.

In addition, for the purpose of increasing the expression of the desired exogenous DNA to a higher level, the splicing signal and enhancer region of each DNA, a portion of the intron of an eukaryotic DNA may also be ligated at the 5′ upstream of the promoter region, or between the promoter region and the translational region, or at the 3′ downstream of the translational region, depending upon purposes.

The translational region for the normal protein of the present invention can be obtained using as a starting material the entire genomic DNA or its portion of liver, kidney, thyroid cell or fibroblast origin from human or various mammals (e.g., rabbits, dogs, cats, guinea pigs, hamsters, rats, mice, etc.) or of various commercially available genomic DNA libraries, or using cDNA prepared by a publicly known method from RNA of liver, kidney, thyroid cell or fibroblast origin as a starting material. Also, an exogenous abnormal DNA can produce the translational region through variation of the translational region of normal protein obtained from the cells or tissues described above by point mutagenesis.

The translational region can be prepared by a conventional DNA engineering technique, in which the DNA is ligated downstream the aforesaid promoter and if desired, upstream the translation termination site, as a DNA construct capable of being expressed in the transgenic animal.

The exogenous DNA of the present invention is transfected at the fertilized egg cell stage in a manner such that the DNA is certainly present in all the germinal cells and somatic cells of the target mammal. The fact that the exogenous DNA of the present invention is present in the germinal cells of the animal prepared by DNA transfection means that all offspring of the prepared animal will maintain the exogenous DNA of the present invention in all of the germinal cells and somatic cells thereof. The offspring of the animal that inherits the exogenous DNA of the present invention also have the exogenous DNA of the present invention in all of the germinal cells and somatic cells thereof.

The non-human mammal in which the normal exogenous DNA of the present invention has been transfected can be passaged as the DNA-bearing animal under ordinary rearing environment, by confirming that the exogenous DNA is stably retained by crossing.

By the transfection of the exogenous DNA of the present invention at the fertilized egg cell stage, the DNA is retained to be excess in all of the germinal and somatic cells. The fact that the exogenous DNA of the present invention is excessively present in the germinal cells of the prepared animal after transfection means that the exogenous DNA of the present invention is excessively present in all of the germinal cells and somatic cells thereof. The offspring of the animal that inherits the exogenous DNA of the present invention have excessively the exogenous DNA of the present invention in all of the germinal cells and somatic cells thereof.

It is possible to obtain homozygous animals having the transfected DNA in both homologous chromosomes and breed male and female of the animal so that all the progeny have this DNA in excess.

In a non-human mammal bearing the normal DNA of the present invention, the normal DNA of the present invention has expressed at a high level, and may eventually develop hyperfunction in the function of the protein of the present invention by accelerating the function of endogenous normal DNA. Therefore, the animal can be utilized as a pathologic model animal for such a disease. For example, using the normal DNA transgenic animal of the present invention, it is possible to elucidate the mechanism of hyperfunction in the function of the protein of the present invention and the pathological mechanism of the disease associated with the protein of the present invention and to investigate how to treat these diseases.

Furthermore, since a mammal transfected with the exogenous normal DNA of the present invention exhibits an increasing symptom of the protein of the present invention liberated, the animal is usable for screening test of preventive/therapeutic agents for diseases associated with the protein of the present invention, for example, the preventive/therapeutic agent for cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, etc.

On the other hand, a non-human mammal having the exogenous abnormal DNA of the present invention can be passaged under normal breeding conditions as the DNA-bearing animal by confirming stable retention of the exogenous DNA via crossing. Furthermore, the exogenous DNA of interest can be utilized as a starting material by inserting the DNA into the plasmid described above. The DNA construct with a promoter can be prepared by conventional DNA engineering techniques. The transfection of the abnormal DNA of the present invention at the fertilized egg cell stage is preserved to be present in all of the germinal and somatic cells of the target mammal. The fact that the abnormal DNA of the present invention is present in the germinal cells of the animal after DNA transfection means that all of the offspring of the prepared animal have the abnormal DNA of the present invention in all of the germinal and somatic cells. Such an offspring that passaged the exogenous DNA of the present invention will have the abnormal DNA of the present invention in all of the germinal and somatic cells. A homozygous animal having the introduced DNA on both of homologous chromosomes can be acquired, and by crossing these male and female animals, all the offspring can be bred to retain the DNA.

In a non-human mammal bearing the abnormal DNA of the present invention, the abnormal DNA of the present invention is expressed to a high level, and may eventually develop the function inactive type inadaptability to the protein of the present invention by inhibiting the functions of endogenous normal DNA. Therefore, the animal can be utilized as a pathologic model animal for such a disease. For example, using the abnormal DNA transgenic animal of the present invention, it is possible to elucidate the mechanism of the function inactive type inadaptability to the protein of the present invention and the pathological mechanism of the disease associated with the protein of the present invention and to investigate how to treat the disease.

More specifically, the transgenic animal of the present invention expressing the abnormal DNA of the present invention at a high level is expected to serve as an experimental model to elucidate the mechanism of the functional inhibition (dominant negative effect) of a normal protein by the abnormal protein of the present invention in the function inactive type inadaptability of the protein of the present invention.

Since a mammal bearing the abnormal exogenous DNA of the present invention shows an increased symptom of the protein of the present invention liberated, the animal is also expected to serve for screening test of the protein of the present invention or the preventive/therapeutic agents for the function inactive type inadaptability, for example, the preventive/therapeutic agent for cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, etc.

Other potential applications of two kinds of the DNA transgenic animals of the present invention described above further include, for example:

(i) use as a cell source for tissue culture;
(ii) elucidation of the relation to a peptide that is specifically expressed or activated by the protein of the present invention, by direct analysis of DNA or RNA in tissues of the DNA transgenic animal of the present invention or by analysis of the peptide tissues expressed by the DNA;
(iii) research on the function of cells derived from tissues that are usually cultured only with difficulty, using cells in tissues bearing the DNA cultured by a standard tissue culture technique;
(iv) screening a drug that enhances the functions of cells using the cells described in (iii) above; and,
(v) isolation and purification of the variant protein of the present invention and preparation of an antibody thereto; etc.

Furthermore, clinical conditions of a disease associated with the protein of the present invention, including the function inactive type inadaptability to the protein of the present invention can be determined by using the DNA transgenic animal of the present invention. Also, pathological findings on each organ in a disease model associated with the protein of the present invention can be obtained in more detail, leading to the development of a new method for treatment as well as the research and therapy of any secondary diseases associated with the disease.

It is also possible to obtain a free DNA-transfected cell by withdrawing each organ from the DNA transgenic animal of the present invention, mincing the organ and degrading with a proteinase such as trypsin, etc., followed by establishing the line of culturing or cultured cells. Furthermore, the DNA transgenic animal of the present invention can serve to identify cells capable of producing the protein of the present invention, and to study in association with apoptosis, differentiation or propagation or on the mechanism of signal transduction in these properties to inspect any abnormality therein. Thus, the DNA transgenic animal can provide an effective research material for the protein of the present invention and for investigation of the function and effect thereof.

To develop a medicament for the treatment of diseases associated with the protein of the present invention, including the function inactive type inadaptability to the protein of the present invention, using the DNA transgenic animal of the present invention, an effective and rapid method for screening can be provided by using the method for inspection and the method for quantification, etc. described above. It is also possible to investigate and develop a method for DNA therapy for the treatment of diseases associated with the protein of the present invention, using the DNA transgenic animal of the present invention or a vector capable of expressing the exogenous DNA of the present invention.

### (7) Knockout Animal

The present invention provides a non-human mammal embryonic stem cell bearing the DNA of the present invention inactivated and a non-human mammal deficient in expressing the DNA of the present invention.

Thus, the present invention provides:

(1) a non-human mammal embryonic stem cell in which the DNA of the present invention is inactivated;
(2) the embryonic stem cell according to (1), wherein the DNA is inactivated by introducing a reporter gene (e.g., β-galactosidase gene derived from Escherichia coli);
(3) the embryonic stem cell according to (1), which is resistant to neomycin;
(4) the embryonic stem cell according to (1), wherein the non-human mammal is a rodent;
(5) the embryonic stem cell according to (4), wherein the rodent is mouse;
(6) a non-human mammal deficient in expressing the DNA of the present invention, wherein the DNA is inactivated;
(7) the non-human mammal according to (6), wherein the DNA is inactivated by inserting a reporter gene (e.g., β-galactosidase derived from Escherichia coli) therein and the reporter gene is capable of being expressed under control of a promoter for the DNA of the present invention;
(8) the non-human mammal according to (6), which is a rodent;
(9) the non-human mammal according to (8), wherein the rodent is mouse; and,
(10) a method of screening a compound or its salt that promotes or inhibits the promoter activity to the DNA of the present invention, which comprises administering a test compound to the mammal of (7) and detecting expression of the reporter gene.

The non-human mammal embryonic stem cell in which the DNA of the present invention is inactivated refers to a non-human mammal embryonic stem cell that suppresses the ability of the non-human mammal to express the DNA by artificially mutating the DNA of the present invention, or the DNA has no substantial ability to express the protein of the present invention (hereinafter sometimes referred to as the knockout DNA of the present invention) by substantially inactivating the activities of the protein of the present invention encoded by the DNA (hereinafter merely referred to as ES cell).

As the non-human mammal, the same examples as described above apply.

Techniques for artificially mutating the DNA of the present invention include deletion of a part or all of the DNA sequence and insertion of or substitution with other DNA, by genetic engineering. By these variations, the knockout DNA of the present invention may be prepared, for example, by shifting the reading frame of a codon or by disrupting the function of a promoter or exon.

Specifically, the non-human mammal embryonic stem cell in which the DNA of the present invention is inactivated (hereinafter merely referred to as the ES cell with the DNA of the present invention inactivated or the knockout ES cell of the present invention) can be obtained by, for example, isolating the DNA of the present invention that the desired non-human mammal possesses, inserting a DNA fragment having a DNA sequence constructed by inserting a drug resistant gene such as a neomycin resistant gene or a hygromycin resistant gene, or a reporter gene such as lacZ (β-galactosidase gene) or cat (chloramphenicol acetyltransferase gene), etc. into its exon site thereby to disable the functions of exon, or integrating to a chromosome of the target animal by, e.g., homologous recombination, a DNA sequence that terminates gene transcription (e.g., polyA additional signal, etc.) in the intron between exons, thus inhibiting the synthesis of complete messenger RNA and eventually destroying the gene (hereinafter simply referred to as a targeting vector). The thus-obtained ES cells to the southern hybridization analysis with a DNA sequence on or near the DNA of the present invention as a probe, or to PCR analysis with a DNA sequence on the targeting vector and another DNA sequence near the DNA of the present invention which is not included in the targeting vector as primers, to select the knockout ES cell of the present invention.

The parent ES cells to inactivate the DNA of the present invention by homologous recombination, etc. may be of a strain already established as described above, or may originally be established in accordance with a modification of the known method by Evans and Kaufman described above. For example, in the case of mouse ES cells, currently it is common practice to use ES cells of the 129 strain. However, since their immunological background is obscure, the C57BL/6 mouse or the BDF₁ mouse (F₁ hybrid between C57BL/6 and DBA/2), wherein the low ovum availability per C57BL/6 in the C57BL/6 mouse has been improved by crossing with DBA/2, may be preferably used for the purpose of obtaining a pure line of ES cells with the clear immunological genetic background and for other purposes. The BDF₁ mouse is advantageous in that, when a pathologic model mouse is generated using ES cells obtained therefrom, the genetic background can be changed to that of the C57BL/6 mouse by back-crossing with the C57BL/6 mouse, since its background is of the C57BL/6 mouse, as well as being advantageous in that ovum availability per animal is high and ova are robust.

In establishing ES cells, blastocytes at 3.5 days after fertilization are commonly used. In the present invention, embryos are preferably collected at the 8-cell stage, after culturing until the blastocyte stage, the embryos are used to efficiently obtain a large number of early stage embryos.

Although the ES cells used may be of either sex, male ES cells are generally more convenient for generation of a germ cell line chimera. It is also desirable that sexes are identified as soon as possible to save painstaking culture time.

Methods for sex identification of the ES cell include the method in which a gene in the sex-determining region on the Y-chromosome is amplified by the PCR process and detected. When this method is used, one colony of ES cells (about 50 cells) is sufficient for sex-determination analysis, which karyotype analysis, for example G-banding method, requires about 10⁶ cells; therefore, the first selection of ES cells at the early stage of culture can be based on sex identification, and male cells can be selected early, which saves a significant amount of time at the early stage of culture.

Also, second selection can be achieved by, for example, confirmation of the number of chromosomes by the G-banding method. It is usually desirable that the chromosome number of the obtained ES cells be 100% of the normal number. However, when it is difficult to obtain the cells having the normal number of chromosomes due to physical operations, etc. in the cell establishment, it is desirable that the ES cell is again cloned to a normal cell (e.g., in a mouse cell having the number of chromosomes being 2n=40) after knockout of the gene of the ES cells.

Although the embryonic stem cell line thus obtained shows a very high growth potential, it must be subcultured with great care, since it tends to lose its ontogenic capability. For example, the embryonic stem cell line is cultured at about 37° C. in a carbon dioxide incubator (preferably 5% carbon dioxide and 95% air, or 5% oxygen, 5% carbon dioxide and 90% air) in the presence of LIF (1 to 10000 U/ml) on appropriate feeder cells such as STO fibroblasts, treated with a trypsin/EDTA solution (normally 0.001 to 0.5% trypsin/0.1 to about 5 mM EDTA, preferably about 0.1% trypsin/1 mM EDTA) at the time of passage to obtain separate single cells, which are then plated on freshly prepared feeder cells. This passage is normally conducted every 1 to 3 days; it is desirable that cells be observed at the passage and cells found to be morphologically abnormal in culture, if any, be abandoned.

Where ES cells are allowed to reach a high density in mono-layers or to form cell aggregates in suspension under appropriate conditions, it is possible to differentiate the ES cells to various cell types, for example, pariental and visceral muscles, cardiac muscle or the like [Nature, 292, 154, 1981; Proc. Natl. Acad. Sci. U.S.A., 78, 7634, 1981; Journal of Embryology Experimental Morphology, 87, 27, 1985]. The cells deficient in expression of the DNA of the present invention, which are obtained from the differentiated ES cells of the present invention, are useful for studying the function of the protein of the present invention cytologically.

The non-human mammal deficient in expression of the DNA of the present invention can be identified from a normal animal by measuring the mRNA level in the subject animal by a publicly known method, and indirectly comparing the degrees of expression.

As the non-human mammal, the same examples given above apply.

With respect to the non-human mammal deficient in expression of the DNA of the present invention, the DNA of the present invention can be knockout by transfecting a targeting vector, prepared as described above, to mouse embryonic stem cells or mouse oocytes, and conducting homologous recombination in which a targeting vector DNA sequence, wherein the DNA of the present invention is inactivated by the transfection, is replaced with the DNA of the present invention on a chromosome of a mouse embryonic stem cell or mouse embryo.

The knockout cells with the disrupted DNA of the present invention can be identified by the southern hybridization analysis using as a probe a DNA fragment on or near the DNA of the present invention, or by the PCR analysis using as primers a DNA sequence on the targeting vector and another DNA sequence at the proximal region of other than the DNA of the present invention derived from mouse used in the targeting vector. When non-human mammal stem cells are used, a cell line wherein the DNA of the present invention is inactivated by homologous recombination is cloned; the resulting clones are injected to, e.g., a non-human mammalian embryo or blastocyst, at an appropriate stage such as the 8-cell stage. The resulting chimeric embryos are transplanted to the uterus of the pseudopregnant non-human mammal. The resulting animal is a chimeric animal constructed with both cells having the normal locus of the DNA of the present invention and those having an artificially mutated locus of the DNA of the present invention.

When some germ cells of the chimeric animal have a mutated locus of the DNA of the present invention, an individual, which entire tissue is composed of cells having a mutated locus of the DNA of the present invention can be selected from a series of offspring obtained by crossing between such a chimeric animal and a normal animal, e.g., by coat color identification, etc. The individuals thus obtained are normally deficient in heterozygous expression of the protein of the present invention. The individuals deficient in homozygous expression of the protein of the present invention can be obtained from offspring of the intercross between those deficient in heterozygous expression of the protein of the present invention.

When an oocyte is used, a DNA solution may be injected, e.g., into the prenucleus by microinjection thereby to obtain a transgenic non-human mammal having a targeting vector introduced in its chromosome. From such transgenic non-human mammals, those having a mutation at the locus of the DNA of the present invention can be obtained by selection based on homologous recombination.

As described above, the individuals in which the DNA of the present invention is knockout permit passage rearing under ordinary rearing conditions, after the individuals obtained by their crossing have proven to have been knockout.

Furthermore, the genital system may be obtained and retained by conventional methods. That is, by crossing male and female animals each having the inactivated DNA, homozygous animals having the inactivated DNA in both loci can be obtained. The homozygotes thus obtained may be reared so that one normal animal and two or more homozygotes are produced from a mother animal to efficiently obtain such homozygotes. By crossing male and female heterozygotes, homozygotes and heterozygotes having the inactivated DNA are proliferated and passaged.

The non-human mammal embryonic stem cell, in which the DNA of the present invention is inactivated, is very useful for preparing a non-human mammal deficient in expression of the DNA of the present invention.

Since the non-human mammal deficient in expression of the DNA of the present invention lacks various biological activities derived from the protein of the present invention, such an animal can be a disease model suspected of inactivated biological activities of the protein of the present invention and thus, offers an effective study to investigate the causes for and therapy for these diseases.

### (7a) Method of Screening Compounds Having Therapeutic/Preventive Effects on Disease Caused by Deficiency, Damages, Etc. of DNA of the Present Invention

The non-human mammal deficient in expression of the DNA of the present invention can be employed for screening the compound having a therapeutic/prophylactic effect on diseases caused by deficiency, damages, etc. of the DNA of the present invention.

That is, the present invention provides a method of screening the compound or its salt having therapeutic/preventive effect on diseases caused by deficiency, damages, etc. of the DNA of the present invention such as cancer, which comprises administering a test compound to a non-human mammal deficient in expression of the DNA of the present invention and, observing and measuring changes occurred in the animal.

As the non-human mammal deficient in expression of the DNA of the present invention, which can be employed for the screening method, the same examples as described above apply.

Examples of the test compound include peptides, proteins, antibodies, non-peptide compounds, synthetic compounds, fermentation products, cell extracts, plant extracts, animal tissue extracts, blood plasma, and the like. These compounds may be novel or publicly known compounds. The test compounds may form salts and as salts of the test compounds, there are, for example, metal salts, ammonium salts, salts with organic bases, salts with inorganic acids, salts with organic acids, salts with basic or acidic amino acids, etc. Preferred examples of the metal salts include alkali metal salts such as sodium salts, potassium salts, etc.; alkaline earth meal salts such as calcium salts, magnesium salts, barium salts, etc.; aluminum salts, etc. Preferred examples of the salts with organic bases include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine, etc. Preferred examples of the salts with inorganic acids include salts with hydrochloric acid, hydrobromic acid, nitric acid, sulfuric acid, phosphoric acid, etc. Preferred examples of the salts with organic acids include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid, etc. Preferred examples of the salts with basic amino acids include salts with arginine, lysine, ornithine, etc., and preferred examples of the salts with acidic amino acids include salts with aspartic acid, glutamic acid, etc.

Among these salts, preferred are pharmaceutically acceptable salts. Where the compounds have, for example, acidic functional groups in the compound, the salts include inorganic salts such as alkali metal salts (e.g., sodium salts, potassium salts, etc.), alkaline earth metal salts (e.g., calcium salts, magnesium salts, barium salts, etc.), etc.; ammonium salts, etc.; and where the compounds have basic functional groups in the compound, the salts include salts with inorganic acids such as hydrobromic acid, nitric acid, sulfuric acid, phosphoric acid, etc., or salts with organic acids such as acetic acid, phthalic acid, fumaric acid, oxalic acid, tartaric acid, maleic acid, citric acid, succinic acid, methanesulfonic acid, p-toluenesulfonic acid, etc.

Specifically, the non-human mammal deficient in expression of the DNA of the present invention is treated with a test compound, comparison is made with an intact animal for control and changes in each organ, tissue, disease conditions, etc. of the animal is used as an indicator to assess therapeutic/preventive effects of the test compound.

For treating an animal to be tested with a test compound, for example, oral administration, intravenous injection, etc. are applied, and the treatment can be appropriately selected depending on conditions of the test animal, properties of the test compound, etc. Furthermore, a dose of the test compound to be administered can be appropriately chosen depending on the administration route, nature of the test compound, etc.

For screening of the compound having therapeutic/preventive effects on, e.g., cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), a test compound is administered to the non-human mammal deficient in expression of the DNA of the present invention. Differences in incidence of arteriosclerotic disease or differences in degree of healing from arteriosclerotic disease in the group administered with no test compound are observed in the tissues described above with passage of time.

In the screening method, when a test compound is administered to a test animal and the disease conditions of the test animal are improved by at least about 10%, preferably at least about 30% and more preferably at least about 50%, the test compound can be selected as the compound having therapeutic/preventive effect on the diseases described above.

The compound obtained using the above screening method is a compound selected from the test compounds described above and exhibits therapeutic/preventive effects on diseases caused by deficiency, damages, etc. of the protein of the present invention. Therefore, the compound can be employed as a safe and low toxic drug for the prevention/treatment of these diseases. Furthermore, compounds derived from the compound obtained by the screening described above may also be used as well.

The compound obtained by the screening method may form salts, and the salts for the compound used are the same as those given for the test compound described above.

The medicament comprising the compound or its salt, which is obtained by the above screening method, can be prepared in a manner similar to the method for preparing the medicament comprising the antibody against the protein of the present invention described hereinabove.

Since the pharmaceutical preparation thus obtained is safe and low toxic, it can be administered to human or a mammal (e.g., rat, mouse, guinea pig, rabbit, sheep, swine, bovine, horse, cat, dog, monkey, etc.).

The dose of the compound or its salt may vary depending upon target disease, subject to be administered, route of administration, etc. For example, when the compound or its salt is orally administered for the treatment of lung cancer, the compound or its salt is administered to an adult (as 60 kg body weight) generally in a daily dose of about 0.1 to 100 mg, preferably about 1.0 to 50 mg and more preferably about 1.0 to 20 mg. In parenteral administration, a single dose of the compound may vary depending upon subject to be administered, target disease, etc. When the compound or its salt is administered for the treatment of lung cancer, it is advantageous to administer the compound or its salt to an adult (as 60 kg body weight) in the form of an injectable preparation, in a single dose of about 0.01 to about 30 mg, preferably about 0.1 to about 20 mg and more preferably about 0.1 to about 10 mg per day. For other animal species, the corresponding dose as converted per 60 kg weight can be administered.

(7b) Method of Screening Compounds that Promote or Inhibit the Activity of Promoter to DNA of the Present Invention

The present invention provides a method of screening a compound or its salt that promotes or inhibits the activity of a promoter to the DNA of the present invention, which comprises administering a test compound to a non-human mammal deficient in expression of the DNA of the present invention and detecting the expression of a reporter gene.

In the screening method described above, an animal in which the DNA of the present invention is inactivated by introducing a reporter gene and the reporter gene can be expressed under control of a promoter to the DNA of the present invention is used as the non-human mammal deficient in expression of the DNA of the present invention, which is selected from the aforesaid non-human mammals deficient in expression of the DNA of the present invention.

The same examples of the test compound apply to specific compounds described above.

As the reporter gene, the same specific examples apply to this screening method. Preferably, there are used β-galactosidase (lacZ), soluble alkaline phosphatase gene, luciferase gene and the like.

Since the reporter gene is present under control of a promoter to the DNA of the present invention in the non-human mammal deficient in expression of the DNA of the present invention wherein the DNA of the present invention is substituted with the reporter gene, the activity of the promoter can be detected by tracing the expression of a substance encoded by the reporter gene.

When a part of the DNA region encoding the protein of the present invention is substituted with, e.g., β-galactosidase gene (lacZ) derived from Escherichia coli, β-galactosidase is expressed in a tissue where the protein of the present invention should originally be expressed, instead of the protein of the present invention. Thus, the state of expression of the protein of the present invention can be readily observed in vivo of an animal by staining with a reagent, e.g., 5-bromo-4-chloro-3-indolyl-β-galactopyranoside (X-gal) which is substrate for β-galactosidase. Specifically, a mouse deficient in the protein of the present invention, or its tissue section is fixed with glutaraldehyde, etc. After washing with phosphate buffered saline (PBS), the system is reacted with a staining solution containing X-gal at room temperature or about 37° C. for approximately 30 minutes to an hour. After the β-galactosidase reaction is terminated by washing the tissue preparation with 1 mM EDTA/PBS solution, the color formed is observed. Alternatively, mRNA encoding lacZ may be detected in a conventional manner.

The compound or its salt obtained using the screening method described above are compounds that are selected from the test compounds described above and that promote or inhibit the promoter activity to the DNA of the present invention.

The compound obtained by the screening method above may form salts, and may be used in the form of salts with physiologically acceptable acids (e.g., inorganic acids, etc.) or bases (e.g., alkali metal salts, etc.), preferably in the form of physiologically acceptable acid addition salts. Examples of such salts are salts with inorganic acids (e.g., hydrochloric acid, phosphoric acid, hydrobromic acid, sulfuric acid, etc.), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid, etc.) and the like.

The compound or its salt that promotes or inhibits the promoter activity to the DNA of the present invention can regulate the expression of the protein of the present invention to regulate the functions of the protein. Thus, the compound or its salt is useful as preventive/therapeutic agents for, e.g., cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.), an apoptosis promoter in cancer cells, a cancer cell growth inhibitor, an agent for suppressing the metastasis/relapse of cancer, etc.

In addition, compounds derived from the compound obtained by the screening described above may also be used as well.

The medicament comprising the compound or its salt obtained by the above screening method can be prepared in a manner similar to the method for preparing the medicament comprising the protein of the present invention or a salt thereof described above.

Since the pharmaceutical preparation thus obtained is safe and low toxic, it can be administered to human or a mammal (e.g., rat, mouse, guinea pig, rabbit, sheep, swine, bovine, horse, cat, dog, monkey, etc.).

A dose of the compound or its salt may vary depending on target disease, subject to be administered, route for administration, etc.; when the compound or its salt that inhibits the promoter activity to the DNA of the present invention is orally administered for the treatment of lung cancer, the compound is administered to an adult (as 60 kg body weight) normally in a daily dose of about 0.1 to 100 mg, preferably about 1.0 to 50 mg and more preferably about 1.0 to 20 mg. In parenteral administration, a dose of the compound may vary depending on target disease, subject to be administered, etc. but when the compound or its salt that inhibits the promoter activity to the DNA of the present invention is administered to an adult (as 60 kg body weight) with lung cancer in the form of injectable preparation, it is advantageous to administer the compound intravenously in a daily dose of about 0.01 to about 30 mg, preferably about 0.1 to about 20 mg and more preferably about 0.1 to about 10 mg. For other animal species, the corresponding dose as converted per 60 kg weight can be administered.

As stated above, the non-human mammal deficient in expression of the DNA of the present invention is extremely useful for screening the compound or its salt that promotes or inhibits the promoter activity to the DNA of the present invention and, can greatly contribute to elucidation of causes for various diseases suspected of deficiency in expression of the DNA of the present invention and for the development of preventive/therapeutic agents for these diseases.

In addition, a so-called transgenic animal (gene transferred animal) can be prepared by using a DNA containing the promoter region of the protein of the present invention, ligating genes encoding various proteins at the downstream and injecting the same into oocyte of an animal. It is thus possible to synthesize the protein therein specifically and study its activity in vivo. When an appropriate reporter gene is ligated to the promoter site described above and a cell line that expresses the gene is established, the resulting system can be utilized as the search system for a low molecular compound having the action of specifically promoting or suppressing the in vivo productivity of the protein itself of the present invention.

(8) Preventive/Therapeutic Agent for Cancer Comprising the Compound or its Salts that Inhibit the Activity of the Protein of the Present Invention, Apoptosis Promoter of Cancer Cells, Cancer Cell Growth Inhibitor, or Agent for Suppressing the Metastasis/Relapse of Cancer

The “compound or its salt that inhibits the activity of the protein of the present invention” in the “preventive/therapeutic agent for cancer comprising the compound or its salt that inhibits the activity of the protein of the present invention, apoptosis promoter of cancer cells, cancer cell growth inhibitor, or agent for suppressing the metastasis/relapse of cancer” may be any substance (e.g., a peptide, protein, antibody, non-peptide compound, synthetic compound, fermentation product, cell extract, plant extract, animal tissue extract, blood plasma, and the like), so long as the substance inhibits the activity (e.g., acyl-CoA synthetase activity) of the protein of the present invention.

The “compound or its salt that inhibits the gene expression of the protein of the present invention” in the “compound or its salt that inhibits the gene expression of the protein of the present invention, apoptosis promoter of cancer cells, cancer cell growth inhibitor, or agent for suppressing the metastasis/relapse of cancer” may be any substance (e.g., a peptide, protein, antibody, non-peptide compound, synthetic compound, fermentation product, cell extract, plant extract, animal tissue extract, blood plasma, etc.), so long as the substance inhibits the gene expression of the protein of the present invention.

These agents described above can be prepared into pharmaceutical preparations in a conventional manner.

The compound or its salt that inhibits the activity of the protein of the present invention and the compound or its salt that inhibits the gene expression of the protein of the present invention can be used in combination with a hormonal therapeutic drug, an anticancer agent (e.g., a chemotherapeutic agent, an immunotherapeutic agent or an agent inhibiting the action of cell growth factors or their receptors) (hereinafter referred to as a concomitant drug).

As the “concomitant drug,” the same “concomitant drug” as described in “(1) Screening of drug candidate compound for disease” above can be used as well.

(9) Anticancer Drug Resistance-Improving Agent Comprising the Compound, Etc. that Inhibits the Activity of the Protein of the Present Invention

The “compound or its salt that inhibits the activity of the protein of the present invention” in the “agent for improving anticancer drug resistance comprising the compound or its salt that inhibits the activity of the protein of the present invention” may be any substance (e.g., a peptide, protein, antibody, non-peptide compound, synthetic compound, fermentation product, cell extract, plant extract, animal tissue extract, blood plasma, etc.), so long as the substance inhibits the activity (e.g., acyl-CoA synthetase activity) of the protein of the present invention.

The “compound or its salt that inhibits the gene expression of the protein of the present invention” in the “agent for improving anticancer drug resistance comprising the compound or its salt that inhibits the gene expression of the protein of the present invention” may be any substance (e.g., a peptide, protein, antibody, non-peptide compound, synthetic compound, fermentation product, cell extract, plant extract, animal tissue extract, blood plasma, etc.), so long as the substance inhibits the gene expression of the protein of the present invention.

These agents described above can be prepared into pharmaceutical compositions in a conventional manner.

The protein of the present invention possesses an anticancer drug resistance action. By inhibiting the activity or expression of the protein of the present invention, cell growth suppressing effects of anticancer drugs against cancer cells are enhanced. Thus, the compound or its salt that inhibits the activity of the protein of the present invention and the compound or its salt that inhibits the gene expression of the protein of the present invention are used as low-toxic and safe agents for improving anticancer drug resistance, etc.

The “anticancer drug” described above include the same examples for the “hormonal drug” and “anticancer agent” (e.g., a chemotherapeutic agent, an immunotherapeutic agent or an agent inhibiting the action of cell growth factors or their receptors), etc. described in “(1) Screening of drug candidate compound for disease” above, preferably, a chemotherapeutic agent.

The “anticancer drug resistance” described above includes, for example, p53 mutation-associated anticancer drug resistance.

The antibody against the protein of the present invention, the antisense polynucleotide of the present invention, siRNA or shRNA, etc. can be used as low-toxic and safe agents for improving anticancer drug resistance, as in the compounds described above.

### (10) Screening of Candidate Compound for Agents of Improving Anticancer Drug Resistance

The protein of the present invention has an anticancer drug resistance action. By inhibiting the activity or expression of the protein of the present invention, cell growth suppressing effects of anticancer drugs against cancer cells are enhanced. Thus, the compound or its salt that inhibits the activity of the protein of the present invention and the compound or its salt that inhibits the gene expression of the protein of the present invention are used as low-toxic and safe agents for improving anticancer drug resistance, etc.

Accordingly, the protein of the present invention is also useful as a reagent for anticancer drug resistance-improving medicaments.

For the screening, the screening methods described in “(1) Screening of drug candidate compound for disease” above, etc. are used.

### (11) Diagnostic Marker

The anticancer effects (e.g., promotion of apoptosis in cancer cells, cancer cell growth suppression, etc.) achieved by inhibiting the activity or expression of the protein of the present invention, the anticancer drug resistance-improving effects (e.g., potentiation of cell growth suppressing effects of anticancer drugs against cancer cells, etc.) or the like are suppressed by (over)expression of the protein of the present invention. Therefore, the protein of the present invention and the polynucleotide encoding the protein of the present invention are useful also as diagnostic markers for diagnosis of sensitivities of anticancer drugs having the action of suppressing the activity or expression of the protein of the present invention (e.g, preventive/therapeutic agents for cancer, apoptosis promoters in cancer cells, cancer cell growth inhibitors, agents for suppressing the metastasis/relapse of cancer, etc.), anticancer drug resistance-improving agents, or the like.

Specifically, when a high expression level of the protein of the present invention or the polynucleotide encoding the protein of the present invention is detected, it can be suspected or diagnosed that the sensitivity of anticancer drug, anticancer drug resistance-improving agent, etc. will be low. Further when a low expression level of the protein of the present invention or the polynucleotide encoding the protein of the present invention is detected, it can be expected or diagnosed that the sensitivity of anticancer drug, anticancer drug resistance-improving agent, etc. will be high.

To determine the expression level of the protein of the present invention, the quantification methods described above in “(2) Quantification of the protein of the invention, its partial peptide, or their salts” or the like are employed. To determine the expression level of the polynucleotide encoding the protein of the present invention (e.g., the level of mRNA encoding the protein of the present invention, etc.), northern hybridization using as a probe a nucleic acid containing the entire or a part of the nucleotide sequence of the polynucleotide encoding the protein of the present invention, or PCR using as a primer a nucleic acid containing the entire or a part of the nucleotide sequence of the polynucleotide encoding the protein of the present invention (e.g., DNA encoding the protein of the present invention, etc.), or the like are employed.

In the specification, when bases, amino acids, etc. are denoted by abbreviations, these codes are based on the abbreviations in accordance with the IUPAC-IUB Commission on Biochemical Nomenclature or by the common codes in the art, examples of which are shown below. For amino acids which may have the optical isomer, L form is presented unless otherwise indicated.

DNA: deoxyribonucleic acid

cDNA: complementary deoxyribonucleic acid

A: adenine

T: thymine

G: guanine

C: cytosine

RNA: ribonucleic acid

mRNA: messenger ribonucleic acid

dATP: deoxyadenosine triphosphate

dTTP: deoxythymidine triphosphate

dGTP: deoxyguanosine triphosphate

dCTP: deoxycytidine triphosphate

ATP: adenosine triphosphate

EDTA: ethylenediaminetetraacetic acid

SDS: sodium dodecyl sulfate

Gly: glycine

Ala: alanine

Val: valine

Ile: isoleucine

Ser: serine

Thr: threonine

Cys: cysteine

Met: methionine

Glu: glutamic acid

Asp: aspartic acid

Lys: lysine

Arg: arginine

His: histidine

Phe: phenylalanine

Tyr: tyrosine

Trp: tryptophan

Pro: proline

Asn: asparagine

Gln: glutamine

pGlu: pyroglutamic acid

Sec: selenocysteine

Substituents, protecting groups and reagents generally used in the specification are presented as the codes below.

Me: methyl group

Et: ethyl group

Bu: butyl group

Ph: phenyl group

TC: thiazolidine-4(R)-carboxamido group

Tos: p-toluenesulfonyl

CHO: formyl

Bzl: benzyl

Cl₂-Bzl: 2,6-dichlorobenzyl

Bom: benzyloxymethyl

Z: benzyloxycarbonyl

Cl-Z: 2-chlorobenzyloxycarbonyl

Br-Z: 2-bromobenzyloxycarbonyl

Boc: t-butoxycarbonyl

DNP: dinitrophenol

Trt: trityl

Bum: t-butoxymethyl

Fmoc: N-9-fluorenylmethoxycarbonyl

HOBt: 1-hydroxybenztriazole

HOOBt: 3,4-dihydro-3-hydroxy-4-oxo-1,2,3-benzotriazine

HONB: 1-hydroxy-5-norbornene-2,3-dicarboxylmide

DCC: N,N′-dicyclohexylcarbodiimide

The sequence identification numbers in the sequence listing of the specification indicate the following sequences.

### [SEQ ID NO: 1]

This shows the amino acid sequence of human ACS1.

### [SEQ ID NO: 2]

This shows the nucleotide sequence of cDNA encoding human ACS1.

### [SEQ ID NO: 3]

This shows the amino acid sequence of human ACS3.

### [SEQ ID NO: 4]

This shows the nucleotide sequence of cDNA encoding human ACS3.

### [SEQ ID NO: 5]

This shows the amino acid sequence of human ACS4 isoform 1.

### [SEQ ID NO: 6]

This shows the nucleotide sequence of cDNA encoding human ACS4 isoform 1.

### [SEQ ID NO: 7]

This shows the amino acid sequence of human ACS4 isoform 2.

### [SEQ ID NO: 8]

This shows the nucleotide sequence of cDNA encoding human ACS4 isoform 2.

### [SEQ ID NO: 9]

This shows the amino acid sequence of human ACS5 isoform a.

### [SEQ ID NO: 10]

This shows the nucleotide sequence of cDNA encoding human ACS5 isoform a.

### [SEQ ID NO: 11]

This shows the amino acid sequence of human ACS5 isoform b.

### [SEQ ID NO: 12]

This shows the nucleotide sequence of cDNA encoding human ACS5 isoform b.

### [SEQ ID NO: 13]

This shows the amino acid sequence of human ACS6 isoform a.

### [SEQ ID NO: 14]

This shows the nucleotide sequence of cDNA encoding human ACS6 isoform a.

### [SEQ ID NO: 15]

This shows the amino acid sequence of human ACS6 isoform b.

### [SEQ ID NO: 16]

This shows the nucleotide sequence of cDNA encoding human ACS6 isoform b.

### [SEQ ID NO: 17]

This shows the nucleotide sequence of the primer used in EXAMPLE 4.

### [SEQ ID NO: 18]

This shows the nucleotide sequence of the primer used in EXAMPLE 4.

### EXAMPLES

Hereinafter the present invention is specifically described below with reference to EXAMPLES but is not deemed to be limited thereto.

### Example 1

### Distribution of p53 gene mutations and apoptosome activities in human solid cancer and analysis of assayed apoptosome activities in p53 mutations

In 34 human solid cancer cell lines (NCI-H23, NCI-H522, NCI-H460, A549, DMS273, DMS114, HCC-2998, KM-12, HT-29, HCT-15, LoVo, LS-174T, HCT-116, St-4, MKN-1, MKN-7, MKN-28, MKN-45, MKN-74, NUGC4, U251, SF268, SF295, SNB-75, SNB-78, A172, KG1C, HBC-4, BSY-1, HBC-5, HTB-26, MCF-7, YMB-1 and Mrk-nu1), p53 mutations and apoptosome activities were analyzed. For control, the apoptosome activity was assayed in 5 human normal cell lines and 9 human normal tissue cells.

The p53 gene mutations of NCI-H23, NCI-H522, NCI-H460, A549, HCC-2998, KM-12, HT-29, HCT-15, LoVo, LS-174T, HCT-116, NUGC4, U251, SF268, SF295, SNB-75, A172, KG1C, HTB-26, MCF-7, YMB-1 and Mrk-nu1 cells have already been reported (Cancer Res., 57,4285-4300, 1997; Mol. Carcinog., 19, 243-253, 1997). For the p53 gene mutation in other cancer cell lines, analysis was made by the method of Takahashi et al. (Int. J. Cancer, 89, 92-99, 2000). To determine the apoptosome activity, the cytoplast fraction was extracted from the cells, 10 μM cytochrome c and 1 mM ATP were added to the fraction and the mixture was reacted at 30° C. for 30 minutes, at which the caspase activation was assayed using acetyl-Asp-Glu-Val-Asp-(4-methylcoumaryl-7-amide) as a substrate. The apoptosome activity values were expressed in terms of relative fluorescence units (RFU). Analysis results of the p53 mutation and apoptosome activity in each cell are shown in TABLE 1. Statistical significance was assessed by the Student's T-test. P<0.05 was considered to indicate statistical significance.

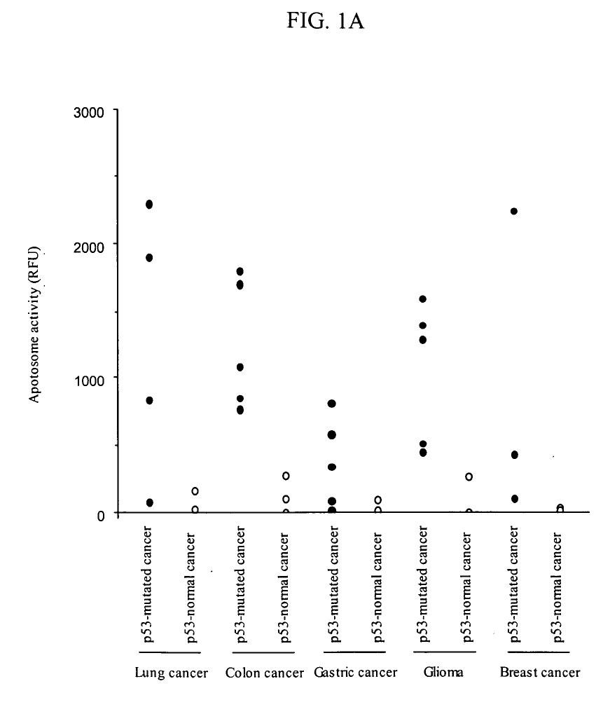

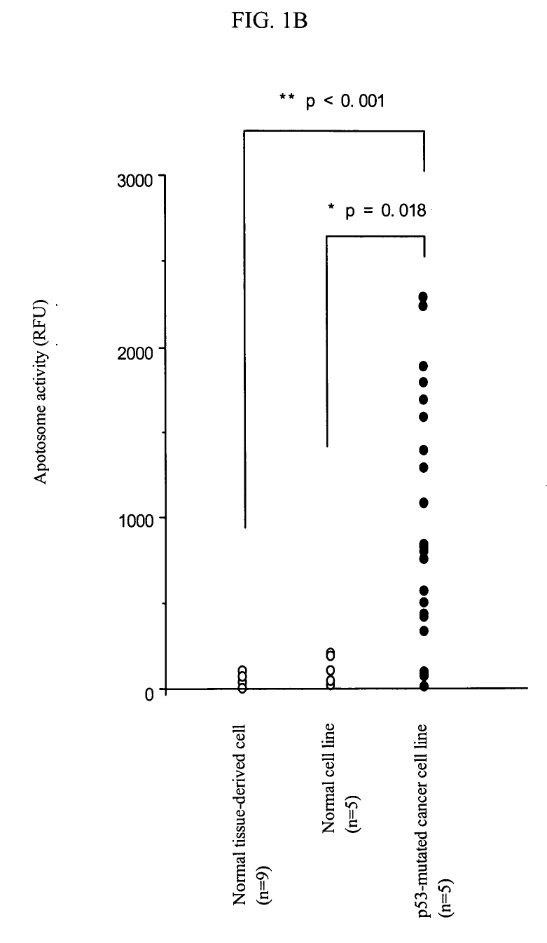

Statistically significant upregulation of the apoptosome activity was noted in the human solid cancer cell lines as compared to the human normal tissue-derived cells (p<0.05). Further when the human solid cancer cell lines were classified into p53-mutated cancers and p53 normal cancers, a complementary distribution was found for the p53 mutations and the low apoptosome activities (FIG. 1A). In particular, the p53-mutated cancers maintained the apoptosome activities downstream of the p53 mutations and produced statistically significant upregulation of the apoptosome activities, as compared to the human normal cell lines and human normal tissue-derived cells (p<0.05) (FIG. 1B).

TABLE 1
Apoptosome activity and p53 gene mutation
in human cancer cells and normal cells

| | | |
|---|---|---|
| Cell | Apoptosome activity, RFU (95% CI) | p53 gene mutation |

Lung cancer

| | | | |
|---|---|---|---|
| NCI-H23 | 840 | (807 to 873) | Mt(M246I) |
| NCI-H522 | 1900 | (1817 to 1983) | Mt(P191del) |
| DMS273 | 88 | (79 to 97) | Mt(G245C) |
| DMS114 | 2300 | (2192 to 2408) | Mt(R213X) |
| NCI-H460 | 170 | (162 to 178) | wild |
| A549 | 30 | (24 to 36) | wild |

Colon cancer

| | | | |
|---|---|---|---|
| HCC-2998 | 870 | (823 to 917) | Mt(R213X) |
| KM-12 | 1700 | (1641 to 1759) | Mt(H179R) |
| HT-29 | 860 | (805 to 915) | Mt(R273H) |
| HCT-15 | 1700 | (1648 to 1752) | Mt(S241F) |
| LoVo | 6.0 | (3.1 to 8.9) | wild |
| LS-174T | 280 | (260 to 300) | wild |
| HCT-116 | 110 | (101 to 119) | wild |

Gastric cancer

| | | | |
|---|---|---|---|
| St-4 | 820 | (813 to 827) | Mt(Y205del) |
| MKN-1 | 590 | (569 to 611) | Mt(V143A) |
| MKN-7 | 350 | (330 to 370) | Mt(I251L) |
| MKN-28 | 25 | (1 to 49) | Mt(I251L) |
| MKN-74 | 97 | (92 to 102) | Mt(I251L) |
| MKN-45 | 100 | (91 to 109) | wild |
| NUGC4 | 22 | (16 to 28) | wild |

Glioma

| | | | |
|---|---|---|---|
| U251 | 1400 | (1273 to 1527) | Mt(R273H) |
| SF268 | 1600 | (1424 to 1776) | Mt(R273H) |
| SF295 | 1300 | (1273 to 1327) | Mt(R248Q) |
| SNB-75 | 450 | (433 to 467) | Mt(E258L) |
| SNB-78 | 520 | (492 to 548) | Mt(184-5del, P223A) |
| A172 | 270 | (269 to 271) | wild |
| KG1C | 6.2 | (6.0 to 6.4) | wild |

Breast cancer

| | | | |
|---|---|---|---|
| BSY-1 | 110.2 | (109.8 to 110.6) | Mt(R248Q) |
| HBC-5 | 430 | (415 to 445) | Mt(C242F) |
| HTB-26 | 2300 | (2218 to 2382) | Mt(R280L) |
| HBC-4 | 16 | (7 to 25) | wild |
| MCF-7 | 9.3 | (9.2 to 9.4) | wild |
| YMB-1 | 21 | (20 to 22) | wild |
| Mrk-nu1 | 23 | (22 to 24) | wild |

Normal cell line

| | | | |
|---|---|---|---|
| TIG108 | 240.0 | (239.4 to 240.6) | n.d. |
| TIG 109 | 240 | (217 to 263) | n.d. |
| ASF4-1 | 88 | (87 to 89) | n.d. |
| CCD33Co | 23 | (22 to 24) | n.d. |
| TIG114 | 23 | (22 to 24) | n.d. |

Normal tissue-derived cell

| | | | |
|---|---|---|---|
| Lung 1 | 1.1 | (1.0 to 1.2) | n.d. |
| Lung 2 | 1.1 | (0.6 to 1.6) | n.d. |
| Lung 3 | 0.9 | (0.8 to 1.0) | n.d. |
| Colon 1 | 9.0 | (6.1 to 11.9) | n.d. |
| Colon 2 | 38 | (0 to 76) | n.d. |
| Colon 3 | 3.0 | (2.2 to 3.8) | n.d. |
| Stomach 1 | 97 | (96 to 98) | n.d. |
| Stomach 2 | 1.0 | (0.97 to 1.03) | n.d. |
| Stomach 3 | 13 | (9 to 17) | n.d. |

In the table, wild, Mt, RFU, Cland n.d. mean wild type, mutant, relative fluorescence units, confidence interval and not determined, respectively.

### Example 2

### Correlation Analysis of the Cell Growth Inhibitory Activity Against Cancer Cells by Acs Inhibitor Triacsin c and the Apoptosome Activity Level of Cancer Cells

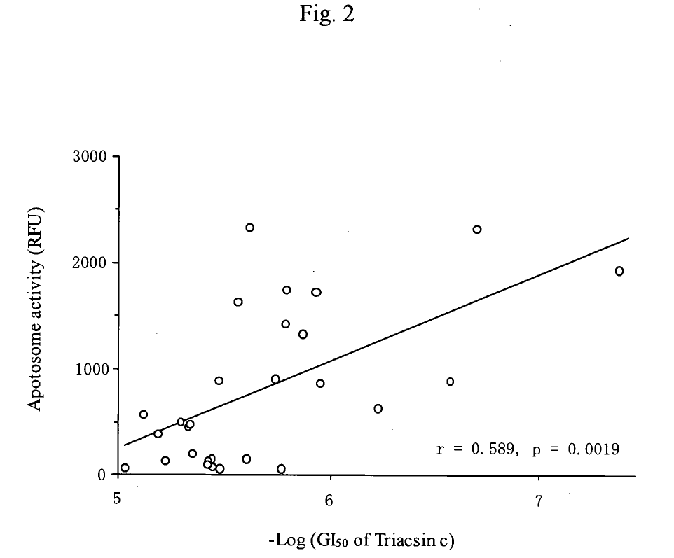

A COMPARE analysis is a computerized analysis approach which involves comparing data for the patterns of drug sensitivities (GI₅₀ values) in a particular set of the cancer cells accumulated in the existing database with data for the pattern of enzyme activity or drug sensitivity (GI₅₀ value) of a test compound and extracting a drug showing the highest correlation (Cancer Chemotherapy Pharmacol., 52, S74-S79, 2003). Using this method, the patterns of apoptosome activities of various cancer cells assayed in EXAMPLE 1 were compared with data for the patterns of drug sensitivities (GI₅₀ values) of 2500 compounds to extract drugs showing strongly positive correlations with the apoptosome activity patterns and drug sensitivity patterns. As a result, Triacsin c, which is a compound having a specific inhibition activity against fatty acid metabolizing enzyme ACS, was extracted (Biochem. Biophys. Acta, 921, 595-598, 1987). The Pearson's correlation coefficient showed a significant correlation of p=0.0019 and r=0.589 between the apoptosome activity of cancer cells and Triacsin c sensitivity (-LOG (GI₅₀)), as shown in FIG. 2.

The results indicate that ACS inhibitor Triacsin c induced cell growth inhibition depending upon the level of apoptosome activity possessed by the cells.

### Example 3

### Analysis of p53-Mutated Cancer-Selective Cell Growth Inhibition and Apoptosis Induction by ACS Inhibitor Triacsin c

Growth inhibition effects on p53-mutated cancer cells and normal cell lines by Triacsin c were analyzed.

NCI-H23, DMS114, HCT-15, KM-12, SF268 and U251 cells, which are p53-mutated cancer cells, were incubated in RPMI-1640 medium containing 4 or 8 μM Triacsin c for 48 hours. On the other hand, human normal cell lines TIG108, TIG109, ASF4-1, CCD33Co and TIG114 cells were incubated in MEM containing 4 or 8 μM Triacsin c for 48 hours. After completion of the incubation, 20 μl of CellTiter 96A_{Queous} One Solution Cell Proliferation Assay Kit (manufactured by Promega Corp.) was added and reacted for an hour. Absorbance was then measured at 490 nm.

The results are shown by % based on absorbance in the absence of Triacsin c.

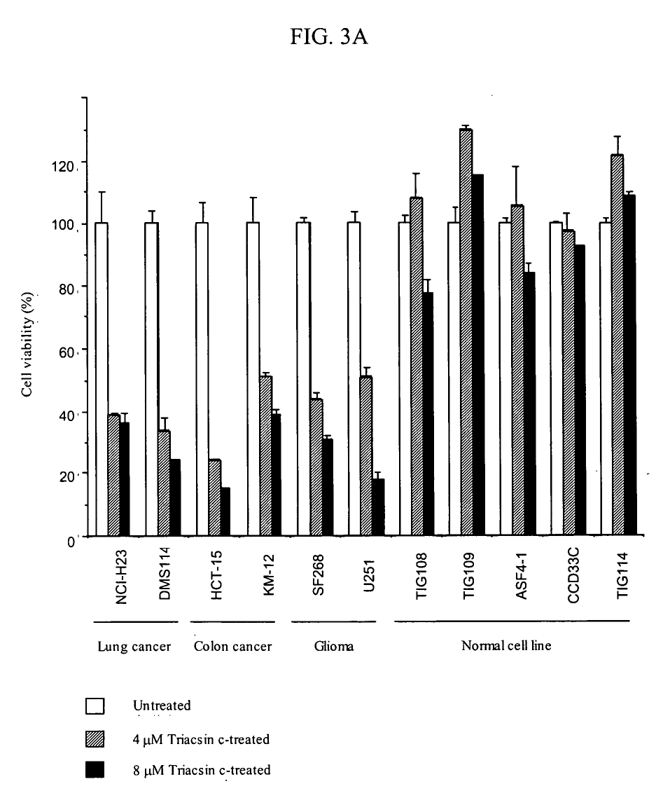

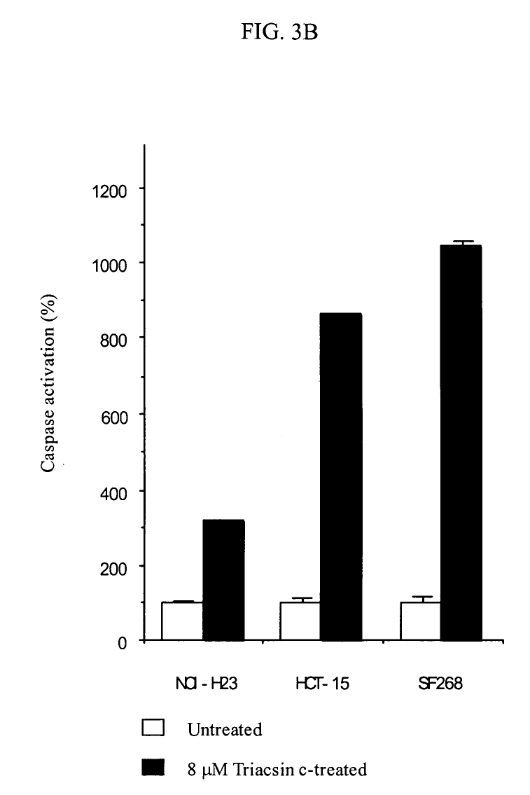

As shown in FIG. 3A, potent cell growth inhibitory effect by Triacsin c was observed in p53-mutated cancers, whereas no cell growth inhibitory effect was observed in human normal cell lines.

In order to examine if the cell growth inhibitory effect by Triacsin c was induced by apoptosis, the caspase activation was measured as an index of the apoptosis induction using acetyl-Asp-Glu-Val-Asp-(4-methylcoumaryl-7-amide) as a substrate.

As a result, strong activation of caspase occurred in the cells incubated for 30 hours in 8 μM Triacsin c-containing RPMI medium as shown in FIG. 3B. This reveals that ACS inhibitor Triacsin c induced p53-mutated cancer cell-selective apoptosis.

### Example 4

### Analysis of Involvement of ACS Activity Inhibition in Apoptosis Induction by Triacsin c

### (1) Cloning of Human ACS5 and Construction of Expression Vector

Human ACS5 gene was cloned by PCR using cDNA from human colon cancer HCT-15 cells. For cloning of human ACS5, two sequences (SEQ ID NO: 17 and SEQ ID NO: 18) were used as primers (to ligate a FLAG tag at the 3′ end, the primers were designed to substitute termination codon TAG for TAT). The reaction was carried out for 35 cycles of 94° C. for 30 seconds, 65° C. for 30 seconds and 72° C. for 4 minutes, using AmpliTaq DNA polymerase (Applied Biosystems). The resulting cDNA fragments were digested at both ends with EcoRI and BamHI (Takara Bio) and then cloned into pFLAG-CMV5 (Cosmo Bio). The pFLAG-CMV-ACS5 obtained was subjected to DNA sequencing. As a result, the cDNA sequence obtained coincided with that of known human ACS5 (AB033899). The obtained ACS5 cDNA was excised out of pFLAG-CMV-ACS5 with FLAG tag at the 3′ end and then incorporated into retroviral vector pHa-IRES-DHFR (Int. J. Cancer, 97, 626-630, 2002) to construct pHa-ACS5—FLAG-IRES-DHFR.

### (2) Establishment of ACS5 Stably Expressing Cell

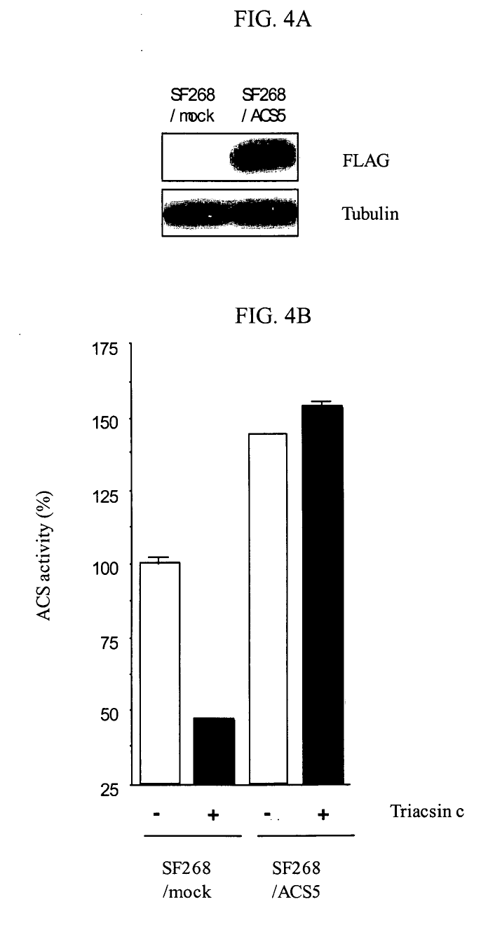

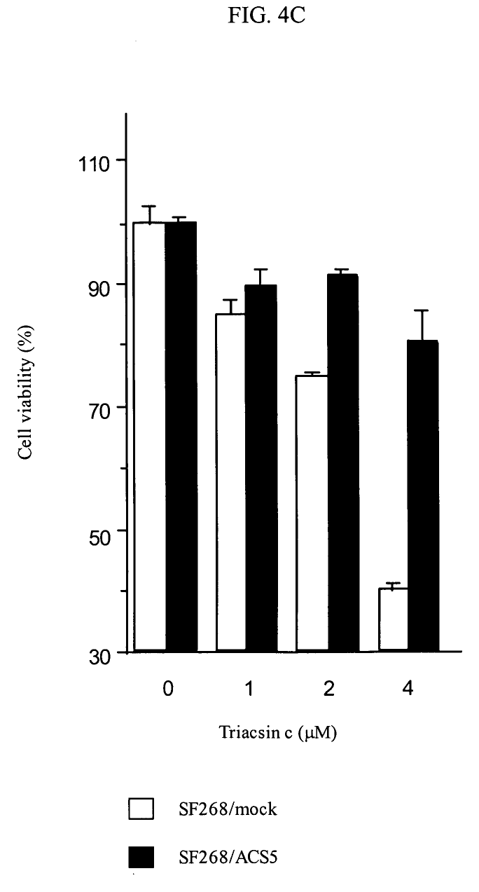

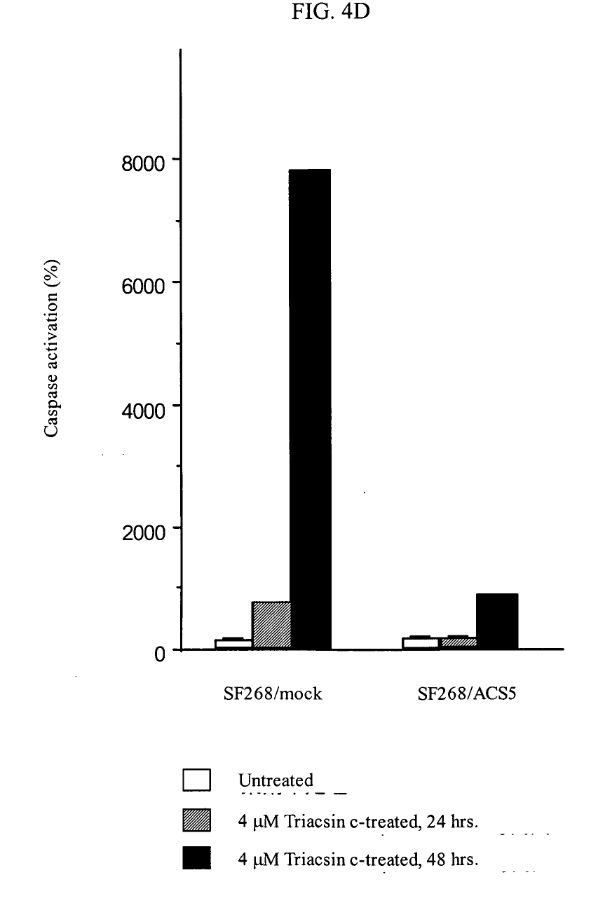

To examine if the p53-mutated cancer cell-selective apoptosis by Triacsin c was induced by ACS activity inhibition, cells stably expressing Triacsin c-resistant ACS isozyme ACS5 were established as follows. The retroviral vector pHa-ACS5—FLAG-IRES-DHFR constructed in EXAMPLE 4(1) and empty vector pHa-IRES-DHFR were introduced into mouse fibroblast PA317 using mammalian transfection kit (Stratagene) to give viral stock. The resulting viral stock was further infected into human glioma SF268 cells and selected with 100 ng/ml of methotrexate as a selective agent to give the stably expressing strain SF268/ACS5 and SF268/mock for control. The cell extract from each cell was analyzed by western blot using anti-FLAG-M2 antibody (Sigma) (and anti-tubulin antibody (Sigma) for control). Stable expression of ACS5 protein was observed in SF268/ACS5 (FIG. 4A).

### (3) Assay of ACS Activity

The ACS activity was assayed by the method of Banis, et al. (Biochem. Biophys. Acta, 348, 210-220, 1974). To the cell extract (100 μl) obtained by homogenization, 1.2 μmol magnesium chloride, 5 μmol ATP, 3 μmol potassium fluoride, 0.1 μmol coenzyme A, 3 μmol 2-mercaptoethanol, 0.03 μmol palmitic acid and 0.1 μCi [¹⁴C]-palmitic acid (150 μl) were added and the mixture was reacted at 37° C. for 5 minutes. After the reaction was terminated by adding 2.25 ml of isopropanol-heptane-1M sulfuric acid (40:10:1), 1.5 ml of heptane and 1 ml of water were added and the upper layer was removed. The lower layer containing [¹⁴C]-palmitoyl CoA produced after the reaction was washed twice with 2 ml of heptane-8 mg of palmitic acid, and the ¹⁴C level was measured using a liquid scintillation counter.

### (4) Inhibition of ACS Activity by Triacsin c and its Counteracting Effect by ACS5 Expression

The ACS activities of SF268/mock cells and SF268/ACS5 cells were assayed by the method described in EXAMPLE 4(3). As shown in FIG. 4B, the intracellular ACS activity was markedly reduced in SF268/mock cells after incubation in 4 μM Triacsin c-containing RPMI-1640 medium for 24 hours. On the other hand, when ACS5-overexpressed SF268/ACS5 cells were examined in a similar manner, any significant reduction of the ACS activity was not observed.

The results reveal that Triacsin c strongly inhibits the ACS activity in SF268 cells and this ACS activity inhibition by Triacsin c is suppressed by overexpression of ACS5.

### (5) Suppression of Triacsin C-Induced Cell Growth Inhibitory Effect by ACS5 Expression

Studies were made to see if the Triacsin c-induced cell growth inhibitory effect was induced by inhibition of the ACS activity.

First, Triacsin c strongly inhibited the growth of SF268 cells under the concentration conditions of about 4 μM (1 to 4 μM, treated for 48 hours) described in EXAMPLE 4(4), at which concentration the ACS activity was inhibited, as shown in FIG. 4C. On the other hand, Triacsin c-induced cell growth inhibitory effect was almost completely cancelled in the cells wherein the Triacsin c-induced ACS activity inhibition-suppressing ACS5 was expressed. In addition, the caspase activation as an index of apoptosis was examined. The caspase activation by Triacsin c was strongly counteracted by the expression of ACS5 (FIG. 4D).

These results demonstrated that the inhibition of ACS activity by Triacsin c induced the apoptosis and growth inhibitory effect.

### Example 5

### Analysis of Growth Inhibitory Effect in Nude Mice Bearing p53-Mutated Cancer by Triacsin c

In order to examine the p53-mutated cancer growth inhibitory effect by apoptosis induction via Triacsin c-induced ACS activity inhibition in vivo, a test for administering p53-mutated cancer-bearing nude mice was performed. Human lung cancer NCI-H23 was used as the p53-mutated cancer cell line and implanted subcutaneously into BALB/cAJcl-nu nude mice (9 weeks old). On day 0 when the tumor volume reached 50-150 mm³, 40 μl (30 mg/kg/day) of Triacsin c was administered by intratumoral injection (n=5). Triacsin c was administered for 3 consecutive days on days 0, 1 and 2. For control group, 40 μl of saline was administered by the same dosing schedule (n=5). For evaluation of tumor volume, the length (L) and width (W) of the tumor were measured, and the tumor volume (TV) was calculated as TV=L×W×W/2. Relative tumor volume was determined as the value when a mean value of the tumor volume on day 0 in control group was made 1. Tumor growth was measured for 21 days after drug administration. Statistical significance was determined with a Student t test.

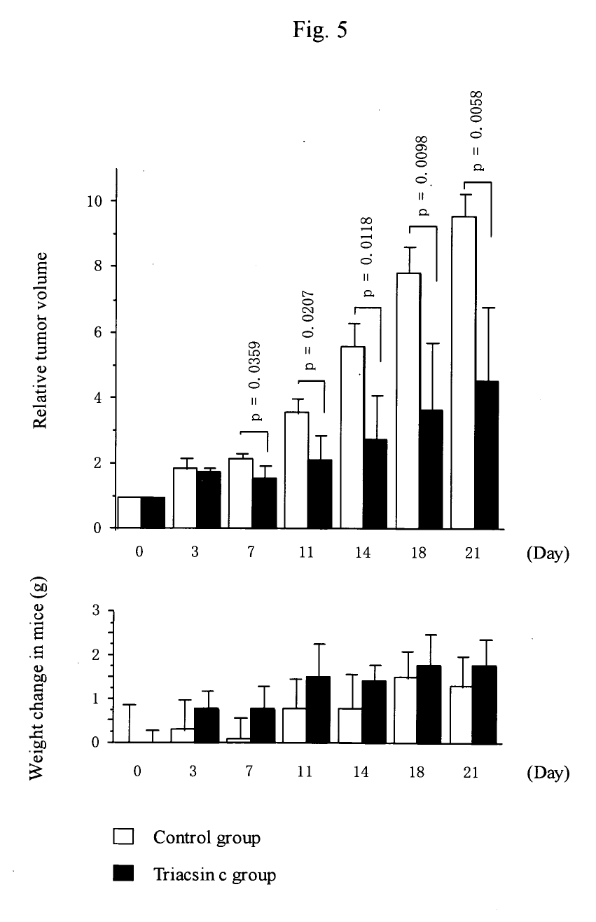

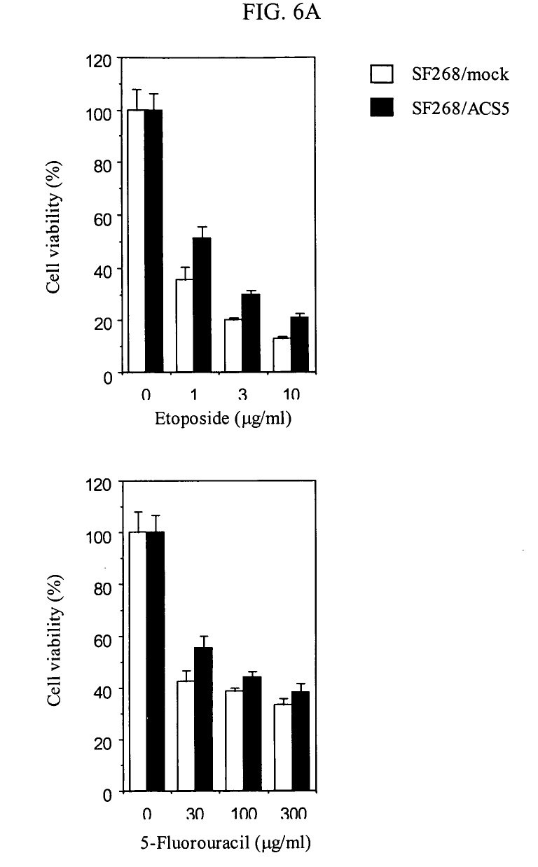

The results are shown in FIG. 5. The Triacsin c group showed a significant tumor regression effect when compared to the control group (p<0.05). In this case, any weight loss was not observed in the cancer-bearing nude mice.

### Example 6

### Emergence of Anticancer Drug Resistance by Expression of ACS and Potentiation of Cell Growth Inhibitory Effect of Anticancer Agent by Inhibition of ACS

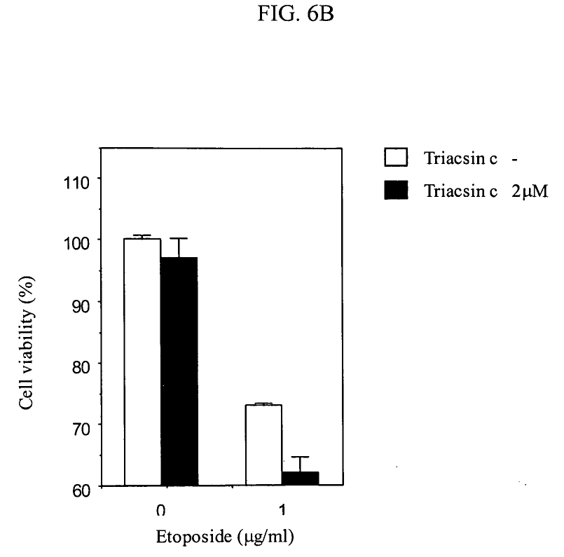

Changes of anticancer drug sensitivity in cancer cells by expression of ACS5 were examined. After 1, 3 or 10 g/ml of etoposide or 30, 100 or 300 g/ml of 5-fluorouracil was added to SF268/ACS5 established in EXAMPLE 4(2), which are cells capable of stably and highly expressing ACS5, and SF268/mock cells for control, incubation was performed in RPMI-1640 medium for 48 hours. After adding 20 μl of CellTiter 96AQᵤₑₒᵤₛ One Solution Cell Proliferation Assay Kit (manufactured by Promega) and reacting for an hour, the cell viability was determined by measuring absorbance at 490 nm and expressed by % based on the value when no etoposide was added or no 5-fluorouracil was added (FIG. 6A).

As shown in FIG. 6A, the growth inhibitory effect was produced concentration-dependently when treated with etoposide or 5-fluorouracil for 48 hours. In this case, significantly high cell viability was noted in ACS5-stably and highly expressing cells SF268/ACS5, as compared to SF268/mock cells for control. This indicates that expression of ACS5 is associated with emergence of anticancer drug resistance in cancer cells.

Furthermore, 1 μg/ml of etoposide and 2 μM Triacsin c were added to SF268 cells. After incubation for 36 hours, the cell viability was determined by the same method as described above. The results are shown in FIG. 6B. Use of etoposide and Triacsin c in combination potentiated the cell growth inhibitory action, as compared to use of etoposide alone. This indicates that inhibition of the ACS activity induces potentiation of the cell growth inhibitory effect of anticancer agent against cancer cells.

### INDUSTRIAL APPLICABILITY

The protein of the present invention is expressed at elevated level in cancer tissues and has an acyl-CoA synthetase activity. By inhibiting the activity or expression of the protein of the present invention, the apoptosome pathway potentiated in cancer cells is activated to induce apoptosis of cancer cells. Accordingly, the compound or its salt that inhibits the activity of the protein of the present invention, the compound or its salt that inhibits the expression of a gene for the protein of the present invention, the antibody against the compound or its salt that inhibits the activity of the protein of the present invention, the antisense polynucleotide of the polynucleotide encoding the protein of the present invention, siRNA, shRNA, etc. can be used as safe pharmaceuticals including, for example, preventive/therapeutic agents for, e.g., cancer (e.g., brain tumor, pituitary tumor, glioma, acoustic neuroma, retinosarcoma, thyroid cancer, pharyngeal cancer, laryngeal cancer, tongue cancer, thymoma, mesothelioma, breast cancer, lung cancer, non-small cell lung cancer, small cell lung cancer, gastric cancer, esophageal cancer, duodenal cancer, colorectal cancer, colon cancer, rectal cancer, liver cancer, hepatocellular carcinoma, pancreatic cancer, pancreatic endocrine tumor, bile duct cancer, gallbladder cancer, penile cancer, renal cancer, renal pelvic cancer, ureteral cancer, renal cell carcinoma, testicular tumor, prostate cancer, bladder cancer, vulvar cancer, uterine cancer, cervical cancer, uterine body cancer, uterine sarcoma, trophoblastic disease, vaginal cancer, ovary cancer, ovarian germ cell tumor, skin cancer, malignant melanoma, mycosis fungoides, basalioma, soft part sarcoma, malignant lymphoma, Hodgkin's disease, myelodysplastic syndrome, multiple myeloma, leukemia, acute myeloid leukemia, chronic myeloid leukemia, acute lymphocytic leukemia, chronic lymphocytic leukemia, adult T-cell leukemia, chronic myeloproliferative disease, pancreatic endocrine tumor, fibrous histiocytoma, leiomyosarcoma, rhabdomyosarcoma, unknown primary cancer, etc.); apoptosis promoters of cancer cells, growth inhibitors of cancer cells, agents for preventing metastasis/relapse of cancer, etc.

In addition, the protein of the present invention has an anticancer drug resistance action. By inhibiting the activity or expression of the protein of the present invention, the cell growth inhibitory effect of anticancer agents against cancer cells is potentiated. Thus, the compound or its salt that inhibits the activity of the protein of the present invention, the compound or its salt that inhibits the expression of a gene for the protein of the present invention, the antibody against the compound or its salt that inhibits the activity of the protein of the present invention, the antisense polynucleotide of the polynucleotide encoding the protein of the present invention, siRNA, shRNA, etc. can be used as safe pharmaceuticals such as agents for improving anticancer drug resistance, etc.

1. A preventive/therapeutic agent for cancer comprising a compound or a salt thereof which inhibits the activity of an enzyme belonging to the acyl-CoA synthetase family.

2. The preventive/therapeutic agent according to claim 1, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

3. The preventive/therapeutic agent according to claim 1, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

4. A preventive/therapeutic agent for cancer comprising a compound or a salt thereof which inhibits the gene expression of an enzyme belonging to the acyl-CoA synthetase family.

5. The preventive/therapeutic agent according to claim 4, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

6. The preventive/therapeutic agent according to claim 4, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

7. The preventive/therapeutic agent according to claims 1 through 6, wherein the cancer is p53-mutated cancer.

8. A preventive/therapeutic agent for cancer, which comprises 1-hydroxy-3-undeca-2,4,7-triene-1-ylidentriaz-1-ene.

9. An antisense polynucleotide comprising an entire or part of a nucleotide sequence complementary or substantially complementary to the nucleotide sequence of a polynucleotide encoding an enzyme belonging to the acyl-CoA synthetase family.

10. The antisense polynucleotide according to claim 9, wherein the enzyme belonging to the acyl-CoA synthetase family is a protein comprising the same or substantially the same amino acid sequence as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

11. A medicament comprising the antisense polynucleotide according to claim 9.

12. A siRNA or shRNA to a polynucleotide encoding an enzyme belonging to the acyl-CoA synthetase family.

13. The siRNA or shRNA according to claim 12, wherein the enzyme belonging to the acyl-CoA synthetase family is a protein comprising the same or substantially the same amino acid sequence as the amino acid sequence represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

14. A medicament comprising the siRNA or shRNA according to claim 12.

15. The medicament according to claim 11 or claim 14, which is a preventive/therapeutic agent for cancer.

16. A preventive/therapeutic agent for cancer comprising an antibody against an enzyme belonging to the acyl-CoA synthetase family.

17. The preventive/therapeutic agent according to claim 16, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

18. A diagnostic agent for cancer comprising an antibody against an enzyme belonging to the acyl-CoA synthetase family.

19. The diagnostic agent according to claim 18, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

20. A diagnostic agent for cancer comprising a polynucleotide encoding an enzyme belonging to the acyl-CoA synthetase family.

21. The diagnostic agent according to claim 20, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

22. A method for diagnosis of cancer, which comprises using an antibody against an enzyme belonging to the acyl-CoA synthetase family or a polynucleotide encoding an enzyme belonging to the acyl-CoA synthetase family.

23. The method for diagnosis according to claim 22, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

24. Use of an enzyme belonging to the acyl-CoA synthetase family as a diagnostic marker of cancer.

25. The use according to claim 24, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

26. A screening method of a medicament for preventing/treating cancer, which comprises using an enzyme belonging to the acyl-CoA synthetase family.

27. The screening method according to claim 26, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

28. The screening method according to claim 26, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 1, its partial peptide, or a salt thereof.

29. The screening method according to claim 26, wherein the cancer is p53-mutated cancer.

30. A screening kit for a medicament for preventing/treating cancer, which comprises an enzyme belonging to the acyl-CoA synthetase family.

31. The screening kit according to claim 30, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

32. A screening method for a medicament for preventing/treating cancer, which comprises using a polynucleotide encoding an enzyme belonging to the acyl-CoA synthetase family.

33. A screening kit for a medicament for preventing/treating cancer, which comprises a polynucleotide encoding an enzyme belonging to the acyl-CoA synthetase family.

34. A screening method of a medicament for preventing/treating cancer, which comprises measuring the amount or activity of an enzyme belonging to the acyl-CoA synthetase family.

35. A method for preventing/treating cancer, which comprises inhibiting the activity of an enzyme belonging to the acyl-CoA synthetase family.

36. The method for preventing/treating cancer according to claim 35, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

37. A method for preventing/treating cancer, which comprises inhibiting the gene expression of an enzyme belonging to the acyl-CoA synthetase family.

38. The preventive/therapeutic method according to claim 37, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

39. A method for preventing/treating cancer, which comprises administering to a mammal an effective dose of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family.

40. (canceled)

41. An agent for promoting the apoptosis of cancer cells or an agent for suppressing the growth of cancer cells, comprising a compound or a salt thereof which inhibits the activity of an enzyme belonging to the acyl-CoA synthetase family.

42. The agent according to claim 41, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

43. The agent according to claim 41, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

44. An agent for promoting the apoptosis of cancer cells or an agent for suppressing the growth of cancer cells, which comprises a compound or a salt thereof which inhibits the gene expression of an enzyme belonging to the acyl-CoA synthetase family.

45. The agent according to claim 44, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

46. The agent according to claim 44, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

47. The agent according to claims 41 through 46, wherein the cancer is p53-mutated cancer.

48. An agent for promoting the apoptosis of cancer cells or an agent for suppressing the growth of cancer cells, which comprises 1-hydroxy-3-undeca-2,4,7-triene-1-ylidenetriaz-1-ene.

49. The medicament according to claim 11 or claim 14, which is an agent for promoting the apoptosis of cancer cells or an agent for suppressing the growth of cancer cells.

50. An agent for promoting the apoptosis of cancer cells or an agent for suppressing the growth of cancer cells, which comprises an antibody against an enzyme belonging to the acyl-CoA synthetase family.

51. The agent according to claim 50, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

52. A screening method of an agent for promoting the apoptosis of cancer cells or an agent for suppressing the growth of cancer cells, which comprises using an enzyme belonging to the acyl-CoA synthetase family.

53. The screening method according to claim 52, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

54. The screening method according to claim 52, wherein the enzyme belonging to the acyl-CoA synthetase family is at least one selected from a protein comprising the same or substantially the same amino acid sequence as the amino acid sequences represented by SEQ ID NO: 5, SEQ ID NO: 7, SEQ ID NO: 9 or SEQ ID NO: 11, its partial peptide, or a salt thereof.

55. The screening method according to claim 52, wherein the cancer is p53-mutated cancer.

56. A screening kit for an agent for promoting the apoptosis of cancer cells or an agent for suppressing the growth of cancer cells, which comprises an enzyme belonging to the acyl-CoA synthetase family.

57. The screening kit according to claim 56, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

58. A screening method of an agent for promoting the apoptosis of cancer cells or an agent for suppressing the growth of cancer cells, which comprises using a polynucleotide encoding an enzyme belonging to the acyl-CoA synthetase family.

59. A screening kit for a medicament for preventing/treating cancer, which comprises polynucleotide encoding enzyme belonging to the acyl-CoA synthetase family.

60. A screening method of an agent for promoting the apoptosis of cancer cells or an agent for suppressing the growth of cancer cells, which comprises measuring the amount or activity of an enzyme belonging to the acyl-CoA synthetase family.

61. A method for promoting the apoptosis of cancer cells or suppressing the growth of cancer cells, which comprises inhibiting the activity of an enzyme belonging to the acyl-CoA synthetase family.

62. The method according to claim 61, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

63. A method for promoting the apoptosis of cancer cells or suppressing the growth of cancer cells, which comprises inhibiting the gene expression of an enzyme belonging to the acyl-CoA synthetase family.

64. The method according to claim 63, wherein the enzyme belonging to the acyl-CoA synthetase family is at least one selected from a protein comprising the same or substantially the same amino acid sequence as the amino acid sequences represented by SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, its partial peptide, or a salt thereof.

65. A method for promoting the apoptosis of cancer cells or suppressing the growth of cancer cells, which comprises administering to a mammal an effective dose of (i) a compound or a salt thereof which inhibits the activity of an enzyme belonging to the acyl-CoA synthetase family, (ii) a compound or a salt thereof which inhibits the gene expression of an enzyme belonging to the acyl-CoA synthetase family, (iii) an antibody against an enzyme belonging to the acyl-CoA synthetase family, or (iv) an antisense polynucleotide comprising an entire or part of a nucleotide sequence complementary or substantially complementary to the nucleotide sequence of a polynucleotide encoding an enzyme belonging to the acyl-CoA synthetase family.

66. (canceled)

67. An anticancer drug resistance improving agent comprising a compound or a salt thereof which inhibits the activity of an enzyme belonging to the acyl-CoA synthetase family.

68. The improving agent according to claim 67, wherein the anticancer drug resistance is p53 mutation-induced anticancer drug resistance.

69. A method of improving anticancer drug resistance, which comprises inhibiting the activity of an enzyme belonging to the acyl-CoA synthetase family.

70. A screening method of a medicament for improving anticancer drug resistance, which comprises using an enzyme belonging to the acyl-CoA synthetase family.

71. Use of an enzyme belonging to the acyl-CoA synthetase family as a diagnostic marker of anticancer drug sensitivity.
